SEQUENCE LISTING (45 sequences)

SEQ ID NO: 1 - DNA, 20 bp, Artificial Sequence
  Description of Artificial Sequence Synthetic primer
gctaccacatccaaggaagg

SEQ ID NO: 2 - DNA, 23 bp, Artificial Sequence
  Description of Artificial Sequence Synthetic probe
cgcaaattacccactcccgaccc

SEQ ID NO: 3 - DNA, 22 bp, Artificial Sequence
  Description of Artificial Sequence Synthetic primer
gcctcgaaagagtcctgtattg

SEQ ID NO: 4 - DNA, 17 bp, Artificial Sequence
  Description of Artificial Sequence Synthetic primer
acagagcctcgcctttg

SEQ ID NO: 5 - DNA, 18 bp, Artificial Sequence
  Description of Artificial Sequence Synthetic probe
agctggcggcgggtgtgg

SEQ ID NO: 6 - DNA, 17 bp, Artificial Sequence
  Description of Artificial Sequence Synthetic primer
ccttgcacatgccggag

SEQ ID NO: 7 - DNA, 19 bp, Artificial Sequence
  Description of Artificial Sequence Synthetic primer
acatcgctcagacaccatg

SEQ ID NO: 8 - DNA, 23 bp, Artificial Sequence
  Description of Artificial Sequence Synthetic probe
ccgttgactccgaccttcacctt

SEQ ID NO: 9 - DNA, 20 bp, Artificial Sequence
  Description of Artificial Sequence Synthetic primer
accagagttaaaagcagccc

SEQ ID NO: 10 - DNA, 24 bp, Artificial Sequence
  Description of Artificial Sequence Synthetic primer
tgggtgtgaaccatgagaagtatg

SEQ ID NO: 11 - DNA, 29 bp, Artificial Sequence
  Description of Artificial Sequence Synthetic probe
cctcaagatcatcagcaatgcctcctgca

SEQ ID NO: 12 - DNA, 20 bp, Artificial Sequence
  Description of Artificial Sequence Synthetic primer
caggggtgctaagcagttgg

SEQ ID NO: 13 - DNA, 20 bp, Artificial Sequence
  Description of Artificial Sequence Synthetic primer
ctctcacctcatcagcagtg

SEQ ID NO: 14 - DNA, 20 bp, Artificial Sequence
  Description of Artificial Sequence Synthetic probe
ccagaggccaccctgaccac

SEQ ID NO: 15 - DNA, 21 bp, Artificial Sequence
  Description of Artificial Sequence Synthetic primer
tcacaactgtagcaatcctgg

SEQ ID NO: 16 - DNA, 21 bp, Artificial Sequence
  Description of Artificial Sequence Synthetic primer
gatgcgaagattcactggatg

SEQ ID NO: 17 - DNA, 28 bp, Artificial Sequence
  Description of Artificial Sequence Synthetic probe
agttctcaaggcagacagtaagctcttc

SEQ ID NO: 18 - DNA, 22 bp, Artificial Sequence
  Description of Artificial Sequence Synthetic primer
tgttgaaccagggatccatatg

SEQ ID NO: 19 - DNA, 17 bp, Artificial Sequence
  Description of Artificial Sequence Synthetic primer
aagccgtagtggggtct

SEQ ID NO: 20 - DNA, 23 bp, Artificial Sequence
  Description of Artificial Sequence Synthetic probe
tataccgctcggttatgctggtg

SEQ ID NO: 21 - DNA, 24 bp, Artificial Sequence
  Description of Artificial Sequence Synthetic primer
aacataaatggcagagattttcca

SEQ ID NO: 22 - DNA, 25 bp, Artificial Sequence
  Description of Artificial Sequence Synthetic primer
aaagactcctaccttattctggatg

SEQ ID NO: 23 - DNA, 27 bp, Artificial Sequence
  Description of Artificial Sequence Synthetic probe
ctgaactgtagaaagggacggaaggac

SEQ ID NO: 24 - DNA, 21 bp, Artificial Sequence
  Description of Artificial Sequence Synthetic primer
cttctacaccaacatccagga

SEQ ID NO: 25 - DNA, 665 bp, Homo sapiens
gggaacacatccaagcttaagacggtgaggtcagcttcacattctcaggaactctccttctttgggtctggctgaagttgaggatctcttactctctaggccacggaattaacccgagcaggcatggaggcctctgctctcacctcatcagcagtgaccagtgtggccaaagtggtcagggtggcctctggctctgccgtagttttgcccctggccaggattgctacagttgtgattggaggagttgtggccatggcggctgtgcccatggtgctcagtgccatgggcttcactgcggcgggaatcgcctcgtcctccatagcagccaagatgatgtccgcggcggccattgccaatgggggtggagttgcctcgggcagccttgtggctactctgcagtcactgggagcaactggactctccggattgaccaagttcatcctgggctccattgggtctgccattgcggctgtcattgcgaggttctactagctccctgcccctcgccctgcagagaagagaaccatgccaggggagaaggcacccagccatcctgacccagcgaggagccaactatcccaaatatacctggggtgaaatataccaaattctgcatctccagaggaaaataagaaataaagatgaattgttgcaactcttcaaaa

SEQ ID NO: 26 - DNA, 656 bp, Homo sapiens
gggaacacatccaagcttaagacggtgaggtcagcttcacattctcaggaactctccttctttgggtctggctgaagttgaggatctcttactctctaggccacggaattaacccgagcaggcatggaggcctctgctctcacctcatcagcagtgaccagtgtggccaaagtggtcagggtggcctctggctctgccgtagttttgcccctggccaggattgctacagttgtgattggaggagttgtggctgtgcccatggtgctcagtgccatgggcttcactgcggcgggaatcgcctcgtcctccatagcagccaagatgatgtccgcggcggccattgccaatgggggtggagttgcctcgggcagccttgtggctactctgcagtcactgggagcaactggactctccggattgaccaagttcatcctgggctccattgggtctgccattgcggctgtcattgcgaggttctactagctccctgcccctcgccctgcagagaagagaaccatgccaggggagaaggcacccagccatcctgacccagcgaggagccaactatcccaaatatacctggggtgaaatataccaaattctgcatctccagaggaaaataagaaataaagatgaattgttgcaactcttcaaaa

SEQ ID NO: 27 - DNA, 1742 bp, Homo sapiens
tctttgaagcttcaaggctgctgaataatttccttctcccattttgtgcctgcctagctatccagacagagcagctaccctcagctctagctgatactacagacagtacaacagatcaagaagtatggcagtgacaactcgtttgacatggttgcacgaaaagatcctgcaaaatcattttggagggaagcggcttagccttctctataagggtagtgtccatggattccgtaatggagttttgcttgacagatgttgtaatcaagggcctactctaacagtgatttatagtgaagatcatattattggagcatatgcagaagagagttaccaggaaggaaagtatgcttccatcatcctttttgcacttcaagatactaaaatttcagaatggaaactaggactatgtacaccagaaacactgttttgttgtgatgttacaaaatataactccccaactaatttccagatagatggaagaaatagaaaagtgattatggacttaaagacaatggaaaatcttggacttgctcaaaattgtactatctctattcaggattatgaagtttttcgatgcgaagattcactggatgaaagaaagataaaaggggtcattgagctcaggaagagcttactgtctgccttgagaacttatgaaccatatggatccctggttcaacaaatacgaattctgctgctgggtccaattggagctgggaagtccagctttttcaactcagtgaggtctgttttccaagggcatgtaacgcatcaggctttggtgggcactaatacaactgggatatctgagaagtataggacatactctattagagacgggaaagatggcaaatacctgccgtttattctgtgtgactcactggggctgagtgagaaagaaggcggcctgtgcagggatgacatattctatatcttgaacggtaacattcgtgatagataccagtttaatcccatggaatcaatcaaattaaatcatcatgactacattgattccccatcgctgaaggacagaattcattgtgtggcatttgtatttgatgccagctctattcaatacttctcctctcagatgatagtaaagatcaaaagaattcgaagggagttggtaaacgctggtgtggtacatgtggctttgctcactcatgtggatagcatggatttgattacaaaaggtgaccttatagaaatagagagatgtgagcctgtgaggtccaagctagaggaagtccaaagaaaacttggatttgctctttctgacatctcggtggttagcaattattcctctgagtgggagctggaccctgtaaaggatgttctaattctttctgctctgagacgaatgctatgggctgcagatgacttcttagaggatttgccttttgagcaaatagggaatctaagggaggaaattatcaactgtgcacaaggaaaaaaatagatatgtgaaaggttcacgtaaatttcctcacatcacagaagattaaaattcagaaaggagaaaacacagaccaaagagaagtatctaagaccaaagggatgtgttttattaatgtctaggatgaagaaatgcatagaacattgtagtacttgtaaataactagaaataacatgatttagtcataattgtgaaaaataataataatttttcttggatttatgttctgtatctgtgaaaaaataaatttcttataaaactcgggtctaaaaaaaaaaaaaaaaaa

SEQ ID NO: 28 - DNA, 5889 bp, Homo sapiens
gctgccagctgagtttttttgctgctttgagtctcagttttctttctttcctagagtctctgaagccacagatctcttaagaactttctgtctccaaaccgtggctgctcgataaatcagacagaacagttaatcctcaatttaagcctgatctaacccctagaaacagatatagaacaatggaagtgacaacaagattgacatggaatgatgaaaatcatctgcgcaagctgcttggaaatgtttctttgagtcttctctataagtctagtgttcatggaggtagcattgaagatatggttgaaagatgcagccgtcagggatgtactataacaatggcttacattgattacaatatgattgtagcctttatgcttggaaattatattaatttacatgaaagttctacagagccaaatgattccctatggttttcacttcaaaagaaaaatgacaccactgaaatagaaactttactcttaaatacagcaccaaaaattattgatgagcaactggtgtgtcgtttatcgaaaacggatattttcattatatgtcgagataataaaatttatctagataaaatgataacaagaaacttgaaactaaggttttatggccaccgtcagtatttggaatgtgaagtttttcgagttgaaggaattaaggataacctagacgacataaagaggataattaaagccagagagcacagaaataggcttctagcagacatcagagactataggccctatgcagacttggtttcagaaattcgtattcttttggtgggtccagttgggtctggaaagtccagttttttcaattcagtcaagtctatttttcatggccatgtgactggccaagccgtagtggggtctgatatcaccagcataaccgagcggtataggatatattctgttaaagatggaaaaaatggaaaatctctgccatttatgttgtgtgacactatggggctagatggggcagaaggagcaggactgtgcatggatgacattccccacatcttaaaaggttgtatgccagacagatatcagtttaattcccgtaaaccaattacacctgagcattctacttttatcacctctccatctctgaaggacaggattcactgtgtggcttatgtcttagacatcaactctattgacaatctctactctaaaatgttggcaaaagtgaagcaagttcacaaagaagtattaaactgtggtatagcatatgtggccttgcttactaaagtggatgattgcagtgaggttcttcaagacaactttttaaacatgagtagatctatgacttctcaaagccgggtcatgaatgtccataaaatgctaggcattcctatttccaatattttgatggttggaaactatgcttcagatttggaactggaccccatgaaggatattctcatcctctctgcactgaggcagatgctgcgggctgcagatgattttttagaagatttgcctcttgaggaaactggtgcaattgagagagcgttacagccctgcatttgagataagttgccttgattctgacatttggcccagcctgtactggtgtgccgcaatgagagtcaatctctattgacagcctgcttcagattttgcttttgttcgttttgccttctgtccttggaacagtcatatctcaagttcaaaggccaaaacctgagaagcggtgggctaagataggtcctactgcaaaccacccctccatatttccgtaccatttacaattcagtttctgtgacatctttttaaaccactggaggaaaaatgagatattctctaatttattcttctataacactctatatagagctatgtgagtactaatcacattgaataatagttataaaattattgtatagacatctgcttcttaaacagattgtgagttctttgagaaacagcgtggattttacttatctgtgtattcacagagcttagcacagtgcctggtaatgagcaagcatacttgccattacttttccttcccactctctccaacatcacattcactttaaatttttctgtatatagaaaggaaaactagcctgggcaacatgatgaaaccccatctccactgcaaaaaaaaaaaaaaaaaataagaaagaacaaaacaaaccccacaaaaattagctgggtatgatggcacgtgcctgtagtcccagttactcaggatgattgattgagccttggaggtggaggctacagtgagctgagattgtgccactgtactctagccagggagaaagagtgagatcctggctcaaaaaaaccaaataaaacaaaacaaacaaacgaaaaacagaaaggaagactgaaagagaatgaaaagctggggagaggaaataaaaataaagaaggaagagtgtttcatttatatctgaatgaaaatatgaatgactctaagtaattgaattaattaaaatgagccaacttttttttaacaatttacattttatttctatgggaaaaaataaatattcctcttctaacaaacccatgcttgattttcattaattgaattccaaatcatcctagccatgtgtccttccatttaggttactggggcaaatcagtaagaaagttcttatatttatgctccaaataattctgaagtcctcttactagctgtgaaagctagtactattaagaaagaaaacaaaattcccaaaagatagctttcacttttttttttccttaaagacttcctaattctcttctccaaattcttagtcttcttcaaaataatatgctttggttcaatagttatccacattctgacagtctaatttagttttaatcagaattatactcatcttttgggtagtcatagatattaagaaagcaagagtttcttatgtccagttatggaatatttcctaaagcaaggctgcaggtgaagttgtgctcaagtgaatgttcaggagacacaattcagtggaagaaattaagtctttaaaaaagacctaggaataggagaaccatggaaattgaggaggtaggcctacaagtagatattgggaacaaaattagagaggcaaccagaaaaagttattttaggctcaccagagttgttcttattgcacagtaacacaccaatataccaaaacagcaggtattgcagtagagaaagagtttaataattgaatggcagaaaaatgaggaaggttgaggaaacctcaaatctacctccctgctgagtctaagtttaggatttttaagagaaaggcaggtaaggtgctgaaggtctggagctgctgatttgttggggtatagggaatgaaatgaaacatacagagatgaaaactggaagtttttttttgtttgttttgttttttttttgttgttgtttttttttttttttgtttttttgctgagtcaattccttggagggggtcttcagactgactggtgtcagcagacccatgggattccaagatctggaaaactttttagatagaaacttgatgtttcttaacgttacatatattatcttatagaaataactaagggaagttagtgccttgtgaccacatctatgtgacttttaggcagtaagaaactataaggaaaggagctaacagtcatgctgtaagtagctacagggaattggcttaaagggcaagttggttagtacttagctgtgtttttattcaaagtctacattttatgtagtggttaatgtttgctgttcattaggatggtttcacagttaccatacaaatgtagaagcaacaggtccaaaaagtagggcatgattttctccatgtaatccagggagaaaacaagccatgaccattgttggttgggagactgaaggtgattgaaggttcaccatcatcctcaccaacttttgggccataattcacccaaccctttggtggagcctgaaaaaaatctgggcagaatgtaggacttctttattttgtttaaaggggtaacacagagtgcccttatgaaggagttggagatcctgcaaggaagagaaggagtgaaggagagatcaagagagagaaacaatgaggaacatttcatttgacccaacatcctttaggagcataaatgttgacactaagttatcccttttgtgctaaaatggacagtattggcaaaatgataccacaacttcttattctctggctctatattgctttggaaacacttaaacatcaaatggagttaaatacatatttgaaatttaggttaggaaatattggtgaggaggcctcaaaaagggggaaacatcttttgtctgggaggatattttccattttgtggatttccctgatctttttctaccaccctgaggggtggtgggaattatcattttgctacattttagaggtcatccaggatttttgaaactttacattctttacggttaagcaagatgtacagctcagtcaaagacactaaattcttcttagaaaaatagtgctaaggagtatagcagatgacctatatgtgtgttggctgggagaatatcatcttaaagtgagagtgatgttgtggagacagttgaaatgtcaatgctagagcctctgtggtgtgaatgggcacgttaggttgttgcattagaaagtgactgtttctgacagaaatttgtagctttgtgcaaactcacccaccatctacctcaataaaatatagagaaaagaaaaatagagcagtttgagttctatgaggtatgcaggcccagagagacataagtatgttcctttagtcttgcttcctgtgtgccacactgcccctccacaaccatagctgggggcaattgtttaaagtcattttgttcccgactagctgccttgcacattatcttcattttcctggaatttgatacagagagcaatttatagccaattgatagcttatgctgtttcaatgtaaattcgtggtaaataacttaggaactgcctcttctttttctttgaaaacctacttataactgttgctaataagaatgtgtattgttcaggacaacttgtctccatacagttgggttgtaaccctcatgcttggcccaaataaactctctacttatatcagtttttcctacacttcttccttttaggtcaacaataccaagaggggttactgtgctgggtaatgtgtaaacttgtgtcttgtttagaaagataaatttaaagactatcacattgctttttcataaaacaagacaggtctacaattaatttattttgacgcaaattgataggggggccaagtaagccccatatgcttaatgatcagctgatgaataatcatctcctagcaacataactcaatctaatgctaaggtacccacaagatggcaaggctgatcaaagtcgtcatggaatcctgcaaccaaaagccatgggaatttggaagccctcaaatcccattcctaatctgatgagtctatggaccaatttgtggaggacagtagattaaatagatctgatttttgccatcaatgtaaggaggataaaaacttgcataccaattgtacacccttgcaaaatctttctctgatgttggagaaaatgggccagtgagatcatggatatagaagtacagtcaatgttcagctgtaccctcccacaatcccacttccttcctcaacacaattcaaacaaatagactcagactgtttcaggctccaggacaggaagtgcagtgtaggcaaaattgcaaaaattgagggcacaggggtggaggtgggggggttgaataacaagctgtgctaaataattacgtgtaaatatattttttcatttttaaaaattgatttcttttgcacattccatgacaatatatgtcacatttttaaaataaatgcaaagaagcatacatccaaaaaaaaaaaaaaaaa

SEQ ID NO: 29 - DNA, 836 bp, Homo sapiens
ccagccttcagccggagaaccgtttactcgctgctgtgcccatctatcagcaggctccgggctgaagattgcttctcttctctcctccaaggtctagtgacggagcccgcgcgcggcgccaccatgcggcagaaggcggtatcgcttttcttgtgctacctgctgctcttcacttgcagtggggtggaggcaggtaagaaaaagtgctcggagagctcggacagcggctccgggttctggaaggccctgaccttcatggccgtcggaggaggactcgcagtcgccgggctgcccgcgctgggcttcaccggcgccggcatcgcggccaactcggtggctgcctcgctgatgagctggtctgcgatcctgaatgggggcggcgtgcccgccggggggctagtggccacgctgcagagcctcggggctggtggcagcagcgtcgtcataggtaatattggtgccctgatgggctacgccacccacaagtatctcgatagtgaggaggatgaggagtagccagcagctcccagaacctcttcttccttcttggcctaactcttccagttaggatctagaactttgcctttttttttttttttttttttttgagatgggttctcactatattgtccaggctagagtgcagtggctattcacagatgcgaacatagtacactgcagcctccaactcctagcctcaagtgatcctcctgtctcaacctcccaagtaggattacaagcatgcgccgacgatgcccagaatccagaactttgtctatcactctccccaacaacctagatgtgaaaacagaataaacttcacccagaaaacactt

SEQ ID NO: 30 - DNA, 848 bp, Homo sapiens
ccagccttcagccggagaaccgtttactcgctgctgtgcccatctatcagcaggctccgggctgaagattgcttctcttctctcctccaaggtctagtgacggagcccgcgcgcggcgccaccatgcggcagaaggcggtatcgcttttcttgtgctacctgctgctcttcacttgcagtggggtggaggcaggtgagaatgcgggtaagaaaaagtgctcggagagctcggacagcggctccgggttctggaaggccctgaccttcatggccgtcggaggaggactcgcagtcgccgggctgcccgcgctgggcttcaccggcgccggcatcgcggccaactcggtggctgcctcgctgatgagctggtctgcgatcctgaatgggggcggcgtgcccgccggggggctagtggccacgctgcagagcctcggggctggtggcagcagcgtcgtcataggtaatattggtgccctgatgggctacgccacccacaagtatctcgatagtgaggaggatgaggagtagccagcagctcccagaacctcttcttccttcttggcctaactcttccagttaggatctagaactttgcctttttttttttttttttttttttgagatgggttctcactatattgtccaggctagagtgcagtggctattcacagatgcgaacatagtacactgcagcctccaactcctagcctcaagtgatcctcctgtctcaacctcccaagtaggattacaagcatgcgccgacgatgcccagaatccagaactttgtctatcactctccccaacaacctagatgtgaaaacagaataaacttcacccagaaaacactt

SEQ ID NO: 31 - DNA, 860 bp, Homo sapiens
ccagccttcagccggagaaccgtttactcgctgctgtgcccatctatcagcaggctccgggctgaagattgcttctcttctctcctccaaggtctagtgacggagcccgcgcgcggcgccaccatgcggcagaaggcggtatcgcttttcttgtgctacctgctgctcttcacttgcagtggggtggaggcaggtgagaatgcgggtaaggatgcaggtaagaaaaagtgctcggagagctcggacagcggctccgggttctggaaggccctgaccttcatggccgtcggaggaggactcgcagtcgccgggctgcccgcgctgggcttcaccggcgccggcatcgcggccaactcggtggctgcctcgctgatgagctggtctgcgatcctgaatgggggcggcgtgcccgccggggggctagtggccacgctgcagagcctcggggctggtggcagcagcgtcgtcataggtaatattggtgccctgatgggctacgccacccacaagtatctcgatagtgaggaggatgaggagtagccagcagctcccagaacctcttcttccttcttggcctaactcttccagttaggatctagaactttgcctttttttttttttttttttttttgagatgggttctcactatattgtccaggctagagtgcagtggctattcacagatgcgaacatagtacactgcagcctccaactcctagcctcaagtgatcctcctgtctcaacctcccaagtaggattacaagcatgcgccgacgatgcccagaatccagaactttgtctatcactctccccaacaacctagatgtgaaaacagaataaacttcacccagaaaacactt

SEQ ID NO: 32 - DNA, 3512 bp, Homo sapiens
aactcagctgagtgttagtcaaagaaggtgtgtcctgctccccaatgacaggttgctcagagactgctgatttccatccctatataaagagagtccctggcatacagagactgctctgctccaggcatctgccacaatgtgggtgcttacacctgctgcttttgctgggaagctcttgagtgtgttcaggcaacctctgagctctctgtggaggagcctggtcccgctgttctgctggctgagggcaaccttctggctgctagctaccaagaggagaaagcagcagctggtcctgagagggccagatgagaccaaagaggaggaagaggaccctcctctgcccaccaccccaaccagcgtcaactatcacttcactcgccagtgcaactacaaatgcggcttctgtttccacacagccaaaacatcctttgtgctgccccttgaggaagcaaagagaggattgcttttgcttaaggaagctggtatggagaagatcaacttttcaggtggagagccatttcttcaagaccggggagaatacctgggcaagttggtgaggttctgcaaagtagagttgcggctgcccagcgtgagcatcgtgagcaatggaagcctgatccgggagaggtggttccagaattatggtgagtatttggacattctcgctatctcctgtgacagctttgacgaggaagtcaatgtccttattggccgtggccaaggaaagaagaaccatgtggaaaaccttcaaaagctgaggaggtggtgtagggattatagagtcgctttcaagataaattctgtcattaatcgtttcaacgtggaagaggacatgacggaacagatcaaagcactaaaccctgtccgctggaaagtgttccagtgcctcttaattgagggtgagaattgtggagaagatgctctaagagaagcagaaagatttgttattggtgatgaagaatttgaaagattcttggagcgccacaaagaagtgtcctgcttggtgcctgaatctaaccagaagatgaaagactcctaccttattctggatgaatatatgcgctttctgaactgtagaaagggacggaaggacccttccaagtccatcctggatgttggtgtagaagaagctataaaattcagtggatttgatgaaaagatgtttctgaagcgaggaggaaaatacatatggagtaaggctgatctgaagctggattggtagagcggaaagtggaacgagacttcaacacaccagtgggaaaactcctagagtaactgccattgtctgcaatactatcccgttggtatttcccagtggctgaaaacctgattttctgctgcacgtggcatctgattacctgtggtcactgaacacacgaataacttggatagcaaatcctgagacaatggaaaaccattaactttacttcattggcttataaccttgttgttattgaaacagcacttctgtttttgagtttgttttagctaaaaagaaggaatacacacaggaataatgaccccaaaaatgcttagataaggcccctatacacaggacctgacatttagctcaatgatgcgtttgtaagaaataagctctagtgatatctgtgggggcaaaatttaatttggatttgattttttaaaacaatgtttactgcgatttctatatttccattttgaaactatttcttgttccaggtttgttcatttgacagagtcagtattttttgccaaatatccagataaccagttttcacatctgagacattacaaagtatctgcctcaattatttctgctggttataatgcttttttttttttgcctttatgccattgcagtcttgtactttttactgtgatgtacagaaatagtcaacagatgtttccaagaacatatgatatgataatcctaccaattttcaagaagtctctagaaagagataacacatggaaagacggtgtggtgcagcccagcccacggtggctgttccatgaatgctggctacctatgtgtgtggtacctgttgtgtccctttctcttcaaagatcctgagcaaaacaaagatacgctttccatttgatgatggagttgacatggaggcagtgcttgcattgctttgttcgcctatcatctggccacatgaggctgtcaagcaaaagaataggagtgtagttgagtagctggttggccctacatctctgagaagtgacggcacactgggttggcataagatatcctaaaatcacgctggaaccttgggcaaggaagaatgtgagcaagagtagagagagtgcctggatttcatgtcagtgaagccaagtcaccatatcatatttttgaatgaactctgagtcagttgaaatagggtaccatctaggtcagtttaagaagagtcagctcagagaaagcaagcataagggaaaatgtcacgtaaactagatcagggaacaaaatcctctccttgtggaaatatcccatgcagtttgttgatacaacttagtatcttattgcctaaaaaaaaatttcttatcattgtttcaaaaaagcaaaatcatggaaaatttttgttgtccaggcaaataaaaggtcattttaatttagctgcaatttcagtgttcctcactaggtggcatttaaatgtcgcctgatgtcattaagcaccatccaaaaagtctgcttcataatctattttcaagacttggtgattctgaaagttttggtttttgtgactttgtttctcaggaaaaaaaatattcctacttaaattttaagtctataattcaatttaaatatgtgtgtgtctcatccaggataggataggttgtcttctattttccattttacctatttactttttttgtaagaaaagagaaaaatgaattctaaagatgttccccatgggttttgattgtgtctaagctatgatgaccttcatataatcagcataaacataaaacaaattttttacttaacatgagtgcactttactaatcctcatggcacagtggctcacgcctgtaatcccagcacttgggaggacaatgtgggtggatcacgaggtcaggagttcgagaacagcctggccaacatggtgaaaccccgtctccactaaaaatacaaaaattagccaggcatggtggcgtacacttgtaattccagctactcaagaggctgaggcaggaggattgcttgaaccctgaaggcagaggttacagagccaagatagcgccactgcactccagcctggatgacagagcaagactccgtctcaaaaaaaaaaaaaaaaaaaagcaagagagttcaactaagaaaggtcacatatgtgaaagcccaaggacactgtttgatatacagcaggtattcaatcagtgttatttgaaaccaaatctgaatttgaagtttgaatcttctgagttggaatgaatttttttctagctgagggaaactgtatttttctttccccaaagaggaatgtaatgtaaagtgaaataaaactataagctatgtt

SEQ ID NO: 33 - DNA, 526 bp, Homo sapiens
gaattcggctttggtgactctagataacctcgggccgatcgcacgccccccgtggcggcgacgacccattcgaacgtctgccctatcaactttcgatggtagtcgccgtgcctaccatggtgaccacggtgacggggaatcagggttcgattccggagagggagcctgagaaacggctccacatccaaggaaggcagcaggcgcgcaaattacccactcccgacccggggaggtagtgacgaaaaataacaatcaggactctttcgaggccctgtaattggaatgagtccactttaaatcctttacgaggatccattggagggcaagtctggtgccagcagccgcggtaattccagctccaatagcgtatattaaagttctgcagttaaaaagctcgtagttggatcttgggagcgggcgggcggtccgccggaggcgagccaccgcccgtccccgccccttgcctctcggcgccccctcgatgctcttagctgagtgtcaaacagaggattacccatgtaagctt

SEQ ID NO: 34 - DNA, 551 bp, Homo sapiens
gaattcaccgccgagaccgcgtccgccccgcgagcacagagcctcgcctttgccgatccgccgcccgtccacacccgccgccagctcaccatggatgatgatatcgccgcgctcgtcgtcgacaacgctccggcatgtgcaaggccggcttcgcgggcgacgatgccccccgggccgtttcccctccatcgtggggcgccccaggcaccagggcgtgatggtgggcatgggtcagaaggattcctatgtgggcacgaggcccagagcaagagaggcatcctcaccctgaagtaccccatcgagcaggcatcgtcaccaactgggacgacatggagaaaatctggcaccacaccttctacaatgagctgcgtgtggctccgaggagcaccccgtgctgctgaccgaggcccccctgaaccccaaggccaaccggagaagatgacccagatcatgtttgagaccttcaacaccccagccatgtacgttgctatccaggctgtgctatcctgtacgcctctggccgtaaacatgaggattacccatgtaagctt

SEQ ID NO: 35 - DNA, 684 bp, Homo sapiens
gaattcaaattgagcccgcagcctcccgcttcgctctctgctcctcctgttcgacagtcagccgcatcttcttttgcgtcgccagccgagccacatcgctcagacaccatggggaaggtgaaggtcgagtcaacggatttggtcgtattgggcgcctggtcaccagggctgcttttaacctggtaaagtggatattgttgccatcaatgaccccttcattgacctcaactacatggtttacatgttccaatatattccacccatggcaaattccatggcaccgtcaaggctgagaacgggaagcttgtcatcaatggaaatcccatcaccatcttccaggagcgagatccctccaaaatcaagtggggcgatgctggcgcagtacgtcgtggagtccactggcgtcttcaccaccatggagaaggctggggctcatttgcaggggggagccaaaagggtcatcatctctgccccctctgctgatgcccccatgttcgtcatgggtggaccatgagaagtatgacaacagcctcaagatcatcagcaatgcctcctgcaccaccaactgcttagcacccctggccaaggtcatccatgacaactttggtatcgtggaaggactcatgaccacatcatgccatcactgccacccagaagaaacatgaggattacccatgtggatcc

SEQ ID NO: 36 - protein, 5 aa, Homo sapiens
NYWIA

SEQ ID NO: 37 - protein, 17 aa, Homo sapiens
IIYPGDSDIRYSPSFQG

SEQ ID NO: 38 - protein, 8 aa, Homo sapiens
HDIEGFDY

SEQ ID NO: 39 - protein, 12 aa, Homo sapiens
RASQSVSSSFFA

SEQ ID NO: 40 - protein, 7 aa, Homo sapiens
GASSRAT

SEQ ID NO: 41 - protein, 9 aa, Homo sapiens
QQYDSSAIT

SEQ ID NO: 42 - protein, 117 aa, Homo sapiens [sequence omitted]

SEQ ID NO: 43 - protein, 108 aa, Homo sapiens [sequence omitted]

SEQ ID NO: 44 - DNA, 351 bp, Homo sapiens
gaggtgcagctggtgcagtctggagcagaggtgaaaaagcccggggagtctctgaagatctcctgtaagggttctggatacatctttaccaactactggatcgcctgggtgcgccagatgcccggtaaaggcctggagtcgatggggatcatctatcctggtgactctgatatcagatacagcccgtccttccaaggccaggtcaccatctcagccgacaagtccatcaccaccgcctacctgcagtggagcagtctgaaggcctcagacaccgccatgtattactgtgcgagacatgacatagaggggtttgactactggggccggggaaccctggtcaccgtctcctca

SEQ ID NO: 45 - DNA, 324 bp, Homo sapiens
gaaattgtgttgacgcagtctccaggcaccctgtctttgtctccaggggaaagagccaccctctcctgcagggccagtcagagtgttagcagcagcttcttcgcctggtaccagcagaaacctggccaggctcccaggctcctcatctatggtgcatccagcagggccactggcatcccagacaggttaagtggcagtgggtctgggacagacttcactctcaccatcaccagactggagcctgaagattttgcagtgtattactgtcagcagtatgatagctcagcgatcaccttcggccaagggacacgactggagattaaa

(19) United States
(12) Patent Application Publication
(10) Pub. No.: US 2016/0024205 A1
(43) Pub. Date: Jan. 28, 2016

(54) Type I Interferon Diagnostic

(71) Applicant: MEDIMMUNE, LLC, Gaithersburg, MD (US)

(72) Inventors: Brandon Higgs, Gaithersburg, MD (US); Wei Zhu, Gaithersburg, MD (US); Chris Morehouse, Middletown, MD (US); Barbara White, Finksburg, MD (US); Bahija Jallal, Potomac, MD (US); Yihong Yao, Boyds, MD (US)

(73) Assignee: MEDIMMUNE, LLC, Gaithersburg, MD (US)

(21) Appl. No.: 14/876,549

(22) Filed: Oct. 6, 2015

Related U.S. Application Data
(63) Continuation of application No. 14/662,134, filed on Mar. 18, 2015.
Application No. 14/662,134 is a continuation of application No. 13/393,616, filed on Jun. 18, 2012, now abandoned.
(60) Provisional application No. 61/239,630, filed on Sep. 3, 2009.

(51) Int. Cl. C07K 16/24; C12Q 1/68

(52) U.S. Cl. CPC C07K 16/249; C12Q 1/6883; C07K 2317/565; C12Q 2600/158; C12Q 2600/106

(57) ABSTRACT

The present disclosure encompasses type-I IFN and IFNα-induced PD marker expression profiles, kits, and methods for identifying such IFNα-induced PD marker expression profiles. The type-I IFN and IFNα-induced PD marker expression profiles may also be used in, for example, methods of treating patients having a type-I IFN or IFNα-mediated disorder, methods of monitoring disease progression of patients receiving treatment with a therapeutic agent that modulates type 1 interferon activity, identifying patients as candidates to receive a therapeutic that binds to and neutralizes IFNα activity, and in diagnosing or providing a prognosis to patients having IFNα-induced disorders.

### CROSS REFERENCE TO RELATED APPLICATIONS

This application claims priority to U.S. Provisional Application No. 61/239,630, filed Sep. 3, 2009, which is incorporated by reference in its entirety.

### SEQUENCE LISTING

The instant application contains a Sequence Listing which has been submitted in ASCII format via EFS-Web and is hereby incorporated by reference in its entirety. The ASCII copy, created on Sep. 1, 2010, is named MED217T4.txt and is 28,665 bytes in size.

### FIELD OF THE DISCLOSURE

The present disclosure relates to pharmacodynamic (PD) markers inducible by type 1 interferons, such as interferon (IFN) alpha, probes and kits that detect the PD markers, and methods employing the same. The present disclosure further relates to PD markers induced by autoimmune disease. The present disclosure further relates to genes whose expression can be used as a diagnostic for patients suffering from autoimmune diseases, such as SLE, DM, PM, SSc, RA, Sjogrens, and lupus nephritis. The disclosure further relates to genes whose expression can be used for identifying patients suffering from an autoimmune disease who will respond to a therapeutic agent that modulates type 1 interferon activity, such as an anti-interferon alpha antibody.

### BACKGROUND OF THE DISCLOSURE

The present disclosure encompasses PD markers that are induced by IFNα. The PD markers can be used in methods of treating patients with a therapeutic agent that a therapeutic agent that modulates type 1 interferon activity, such as an agent that binds to and modulates IFNα activity, methods that identify patients as candidates for a therapeutic agent that modulates type 1 interferon activity, methods of diagnosing a patient as having a disorder associated with increased type 1 interferon or IFNα levels, methods of monitoring disease progression of a patient receiving treatment with a therapeutic agent that modulates type 1 interferon activity, such as a therapeutic agent that binds to and modulates IFNα activity, and methods of identifying a candidate therapeutic for treating IFNα-mediated disorders. The present disclosure also encompasses PD markers otherwise involved in autoimmune diseases such as SLE, DM, PM, SSc, RA, Sjogrens, and lupus nephritis.

### SUMMARY OF THE DISCLOSURE

One embodiment of the disclosure encompasses a method of identifying a patient as a candidate for a therapeutic agent that modulates type 1 interferon activity, such as a therapeutic agent that binds to and modulates IFNα activity and an agent that binds to a receptor of a type I interferon or IFNα. Presence or absence of an IFNα-inducible PD marker expression profile is detected in a sample from the patient. The PD marker expression profile comprises up-regulation of expression or activity of a set of genes. In a specific embodiment, the set of genes may be: (a) IFI27, IFI44, IFI44L, IFI6 and RSAD2; or (b) IFI44, IFI44L, IFI6 and RSAD2; or (c) IFI27, IFI44L, IFI6 and RSAD2; or (d) IFI27, IFI44, IFI6 and RSAD2; or (e) IFI27, IFI44, IFI44L, and RSAD2; or (f) IFI27, IFI44, IFI44L, and IFI6.

Another embodiment of the disclosure encompasses a method of treating a patient having a type I IFN or IFNα-mediated disease or disorder. An agent that modulates type I IFN or IFNα activity is administered to the patient. In one embodiment, the agent binds to and neutralizes IFN or IFNα activity. In another embodiment, the agent binds to a receptor of a type 1 interferon or IFNα. The agent neutralizes a type I IFN or IFNα-inducible PD marker expression profile of the patient. The PD marker expression profile comprises up-regulation of expression or activity of a set of genes. In a specific embodiment, the set of genes may be: (a) IFI27, IFI44, IFI44L, IFI6 and RSAD2; or (b) IFI44, IFI44L, IFI6 and RSAD2; or (c) IFI27, IFI44L, IFI6 and RSAD2; or (d) IFI27, IFI44, IFI6 and RSAD2; or (e) IFI27, IFI44, IFI44L, and RSAD2; or (f) IFI27, IFI44, IFI44L, and IFI6.

Yet another embodiment of the disclosure encompasses a method of treating an autoimmune disease patient comprising a moderate or strong type I IFN or an IFNα PD marker profile. An agent that modulates type I IFN or IFNα activity is administered to the patient. In one embodiment, the agent binds to and neutralizes IFN or IFNα activity. In another embodiment, the agent binds to a receptor of a type 1 interferon or IFNα. The agent neutralizes a type I IFN or IFNα-inducible PD marker expression profile of the patient. The PD marker expression profile comprises up-regulation of expression or activity of a set of genes. In a specific embodiment, the set of genes may be: (a) IFI27, IFI44, IFI44L, IFI6 and RSAD2; or (b) IFI44, IFI44L, IFI6 and RSAD2; or (c) IFI27, IFI44L, IFI6 and RSAD2; or (d) IFI27, IFI44, IFI6 and RSAD2; or (e) IFI27, IFI44, IFI44L, and RSAD2; or (f) IFI27, IFI44, IFI44L, and IFI6.

A further embodiment of the disclosure encompasses a method of neutralizing a type I IFN or IFNα-inducible PD marker expression profile in a patient in need thereof. An agent that modulates type I IFN or IFNα activity is administered to the patient. In one embodiment, the agent binds to and neutralizes IFN or IFNα activity. In another embodiment, the agent binds to a receptor of a type 1 interferon or IFNα. The agent neutralizes a type I IFN or IFNα-inducible PD marker expression profile of the patient. The PD marker expression profile comprises up-regulation of expression or activity of a set of genes. In a specific embodiment, the set of genes may be: (a) IFI27, IFI44, IFI44L, IFI6 and RSAD2; or (b) IFI44, IFI44L, IFI6 and RSAD2; or (c) IFI27, IFI44L, IFI6 and RSAD2; or (d) IFI27, IFI44, IFI6 and RSAD2; or (e) IFI27, IFI44, IFI44L, and RSAD2; or (f) IFI27, IFI44, IFI44L, and IFI6.

Another embodiment of the disclosure encompasses a method of diagnosing a patient as having a disorder associated with increased IFNα levels. Presence or absence of an IFNα-inducible PD marker expression profile is detected in a sample from the patient. The PD marker expression profile comprises up-regulation of expression or activity of a set of genes. In a specific embodiment, the set of genes may be: (a) IFI27, IFI44, IFI44L, IFI6 and RSAD2; or (b) IFI44, IFI44L, IFI6 and RSAD2; or (c) IFI27, IFI44L, IFI6 and RSAD2; or (d) IFI27, IFI44, IFI6 and RSAD2; or (e) IFI27, IFI44, IFI44L, and RSAD2; or (f) IFI27, IFI44, IFI44L, and IFI6.

A further embodiment of the disclosure encompasses a method of monitoring disease progression of a patient receiving treatment with a therapeutic agent that binds to and modulates IFNα activity. A first IFNα-inducible PD marker expression profile is obtained in a first sample from the patient. An agent that modulates type I IFN or IFNα activity is administered to the patient. In one embodiment, the agent binds to and neutralizes IFN or IFNα activity. In another embodiment, the agent binds to a receptor of a type 1 interferon or IFNα. A second IFNα-inducible PD marker expression profile is obtained from a second sample from the patient. The first and the second IFNα-inducible PD marker expression profiles are compared. The PD marker expression profile comprises up-regulation of expression or activity of a set of genes. In a specific embodiment, the set of genes may be: (a) IFI27, IFI44, IFI44L, IFI6 and RSAD2; or (b) IFI44, IFI44L, IFI6 and RSAD2; or (c) IFI27, IFI44L, IFI6 and RSAD2; or (d) IFI27, IFI44, IFI6 and RSAD2; or (e) IFI27, IFI44, IFI44L, and RSAD2; or (f) IFI27, IFI44, IFI44L, and IFI6.

Yet another embodiment of the disclosure encompasses a method of identifying a candidate therapeutic for treating IFNα-mediated disorders. Cells comprising an IFNα-inducible PD marker expression profile are contacted with an agent. Presence or absence of a change in the IFNα-induced PD marker expression profile of the cells is detected. The PD marker expression profile comprises up-regulation of expression or activity of a set of genes. In a specific embodiment, the set of genes may be: (a) IFI27, IFI44, IFI44L, IFI6 and RSAD2; or (b) IFI44, IFI44L, IFI6 and RSAD2; or (c) IFI27, IFI44L, IFI6 and RSAD2; or (d) IFI27, IFI44, IFI6 and RSAD2; or (e) IFI27, IFI44, IFI44L, and RSAD2; or (f) IFI27, IFI44, IFI44L, and IFI6.

A further embodiment of the disclosure encompasses a set of oligonucleotides. The set of oligonucleotides may comprise oligonucleotides that specifically detect expression of a set of genes. In a specific embodiment, the set of genes may be: (a) IFI27, IFI44, IFI44L, IFI6 and RSAD2; or (b) IFI44, IFI44L, IFI6 and RSAD2; or (c) IFI27, IFI44L, IFI6 and RSAD2; or (d) IFI27, IFI44, IFI6 and RSAD2; or (e) IFI27, IFI44, IFI44L, and RSAD2; or (f) IFI27, IFI44, IFI44L, and IFI6.

Yet a further embodiment of the disclosure encompasses oligonucleotides that specifically detect 18S, ACTB, and GAPDH.

Another embodiment of the disclosure is a kit comprising oligonucleotides for specifically detecting at least four of IFI27, IFI44, IFI44L, IFI6 and RSAD2, and 18S, ACTB, and GAPDH, as well as reagents suitable for the detection.

Another embodiment of the disclosure encompasses a method of detecting IFN activity in a sample. Cells comprising a polynucleotide sequence comprising a reporter gene under the control of an IFN-stimulated response element are incubated with a sample. Expression of the reporter gene is detected.

Yet a further embodiment of the disclosure encompasses a method of monitoring autoimmune disorder progression or regression of a patient. A first PD marker expression profile is obtained from a first sample from the patient. A second PD marker expression profile is obtained from a second sample from the patient. The first and the second PD marker expression profiles are compared. A variance in the first and the second PD marker expression profiles indicates disease progression or regression.

### BRIEF DESCRIPTION OF THE FIGURES

FIG. 1. Receiver operator characteristic (ROC) curve for the four gene (IFI27, IFI44, IFI44L, and RSAD2) signature used for a diagnostic. This plot demonstrates the trade off between sensitivity (true positive rate) and 1-specificity (false positive rate) for treated SLE patients using the primary clinical endpoint at days 182 and 196.

FIGS. 2 A and B. Clear boundary between diagnostic test positive and negative patients in SLE using the four gene (IFI27, IFI44, IFI44L, and RSAD2) signature based test. (A) Average log₂ fold change—using Applied Biosystem's qRT-PCR TaqMan low density array (TLDA) platform for test negative and test positive patients is shown. (B) Average log₂ fold change density plot of the gene signature values for drug-treated SLE patients.

FIGS. 3 A and B. Time adjusted area under the curve minus baseline SLEDAI score in four gene (IFI27, IFI44, IFI44L, and RSAD2) signature (A) positive or (B) negative SLE patients in placebo or 0.3/1/3/10 mg/kg of MEDI-545 cohorts. All data are from a phase 1b, multicenter, randomized, double-blinded, placebo-controlled, dose-escalation study to evaluate multiple intravenous doses of MEDI-545 in patients with moderately to severely active SLE. All SLE subjects have SLEDAI score ≧6 at prescreening.

FIGS. 4 A and B. SELDAI responses, reduction (improvement) ≧4 points. A. Diagnostic positive. B. Diagnostic negative. All data are from a phase 1b, multicenter, randomized, double-blinded, placebo-controlled, dose-escalation study to evaluate multiple intravenous doses of MEDI-545 in patients with moderately to severely active SLE. All SLE subjects have SLEDAI score ≧6 at prescreening.

FIGS. 5 A and B. SLEDAI responses, reduction (improvement) >4 points, in Dx+ subjects with >50% reduction vs. <50% reduction in Dx post baseline. All data are from a phase 1b, multicenter, randomized, double-blinded, placebo-controlled, dose-escalation study to evaluate multiple intravenous doses of MEDI-545 in patients with moderately to severely active SLE. All SLE subjects have SLEDAI score ≧6 at prescreening.

FIGS. 6 A and B. Composite Responses. A) Composite response for patients positive for four gene signature. B) Composite response for patients negative for four gene signature. All data are from a phase 1b, multicenter, randomized, double-blinded, placebo-controlled, dose-escalation study to evaluate multiple intravenous doses of MEDI-545 in patients with moderately to severely active SLE. All SLE subjects have SLEDAI score ≧6 at prescreening.

FIGS. 7 A and B. SLEDAI area under the curve minus baseline. A) subjects positive for four gene signature. B) subjects negative for four gene signature. All data are from a phase 1b, multicenter, randomized, double-blinded, placebo-controlled, dose-escalation study to evaluate multiple intravenous doses of MEDI-545 in patients with moderately to severely active SLE. All SLE subjects have SLEDAI score ≧6 at prescreening.

FIGS. 8 A and B. SLEDAI change from baseline. A) subjects positive for four gene signature. B) subjects negative for four gene signature. All data are from a phase 1b, multicenter, randomized, double-blinded, placebo-controlled, dose-escalation study to evaluate multiple intravenous doses of MEDI-545 in patients with moderately to severely active SLE. All SLE subjects have SLEDAI score ≧6 at prescreening.

FIGS. 9 A-C. Subjects with >50% inhibition of Type I IFN Signature have higher SLEDAI responses (reduction >4 points). A) 1 mg/kg IV q2wks B) 3 mg/kg IV q2wks C) 10 mg/kg IV q2wks

FIGS. 10 A-C. Subjects with <50% inhibition of Type I IFN Signature have lower SLEDAI responses (reduction >4 points). A) 1 mg/kg IV q2wks B) 3 mg/kg IV q2wks C) 10 mg/kg IV q2wks.

FIGS. 11 A and B. Five gene signature in various diseases. A) Gene signature in whole blood from normal, SLE, SM, PM, RA, and SSc. B) Gene signature in normal skin, SLE skin, SSc skin, normal muscle, DM muscle, PM muscle, normal synovium tissue.

FIGS. 12 A and B. Four gene signature in various diseases. A) Gene signature in whole blood from normal, SLE, SM, PM, RA, and SSc. B) Gene signature in normal skin, SLE skin, SSc skin, normal muscle, DM muscle, PM muscle, normal synovium tissue.

### DETAILED DESCRIPTION

The disclosure encompasses methods of identifying, diagnosing, treating, and monitoring disease progression in patients. Patients include any animal having a type I IFN or an IFNα-inducible disease, disorder, or condition. Patients include any animal having an autoimmune disease or disorder or condition. Autoimmune diseases/disorders/conditions include systemic lupus erythematosus (SLE), insulin dependent diabetes mellitus, inflammatory bowel disease (including Crohn's disease, ulcerative colitis, and Celiac's disease), multiple sclerosis, psoriasis, autoimmune thyroiditis, schleroderma, rheumatoid arthritis, glomerulonephritis, idiopathic inflammatory myositis, Sjogren's syndrome, vasculitis, inclusion body myositis (IBM), dermatomyositis (DM), polymyositis (PM), sarcoidosis, scleroderma and lupus nephritis. The patient may have the disease, disorder, or condition as a result of experimental research, e.g., it may be an experimental model developed for the disease, disorder, or condition. Alternatively, the patient may have the disease, disorder, or condition in the absence of experimental manipulation. Patients include humans, mice, rats, horses, pigs, cats, dogs, and any animal used for research.

The patient may comprise a type I IFN or IFNα-inducible PD marker expression profile. The type I IFN or IFNα-inducible PD marker expression profile may be a strong profile, a moderate profile, or a weak profile. The type I IFN or IFNα-inducible PD marker expression profile can readily be designated as strong, moderate, or weak by determining the fold dysregulation of the type I IFN or IFNα-inducible PD marker expression profile of the patient, (e.g., the fold increase in expression of upregulated type I IFN or IFNα-inducible PD markers in the patient), relative to a control sample(s) or control patient(s) and comparing the patient's fold dysregulation to that of other patients having a type I IFN or IFNα-inducible PD marker expression profile. Fold dysregulation can be calculated by well known methods in the art as can the comparing. See, e.g., Example 8 of International Application No. PCT/US2007/024947. In one embodiment, the fold dysregulation is calculated as fold change in mRNA expression levels. Strong, moderate, or weak profiles may likewise be generated for genes that are not specifically type I IFN or IFNα-inducible.

Up or down regulation of a group of genes comprised by a type I IFN or IFNα-inducible PD marker expression profile can be calculated by well known methods in the art. In one embodiment, up or down regulation is calculated as average fold change in the mRNA expression levels of the group of at least four genes chosen from IFI27, IFI44, IFI44L, IFI6 and RSAD2. In another embodiment, the up or down regulation is calculated as the difference between the mean Ct (cycle threshold) for at least four target genes (IFI27, IFI44, IFI44L, IFI6 and RSAD2) and the mean Ct of three control genes.

### I. Type I IFN or IFNα-Inducible PD Marker Expression Profile

A. Diagnostic Genes

The group of genes included in the type I IFN or IFNα-inducible PD marker expression profile of the patient are (a) IFI27, IFI44, IFI44L, IFI6 and RSAD2; or (b) IFI44, IFI44L, IFI6 and RSAD2; or (c) IFI27, IFI44L, IFI6 and RSAD2; or (d) IFI27, IFI44, IFI6 and RSAD2; or (e) IFI27, IFI44, IFI44L, and RSAD2; or (f) IFI27, IFI44, IFI44L, and IFI6.

In a specific embodiment, the group of genes included in the type I IFN or IFNα-inducible PD marker expression profile of the patient comprises IFI27, IFI44, IFI44L, IFI6 and RSAD2. In another specific embodiment, the group of genes included in the type I IFN or IFNα-inducible PD marker expression profile of the patient consists of IFI27, IFI44, IFI44L, IFI6 and RSAD2. In a further specific embodiment, the group of genes included in the type I IFN or IFNα-inducible PD marker expression profile of the patient comprises IFI27, IFI44, IFI44L, and RSAD2. In another specific embodiment, the group of genes included in the type I IFN or IFNα-inducible PD marker expression profile of the patient consists of IFI27, IFI44, IFI44L, and RSAD2.

The IFNα-inducible PD markers in an expression profile may include (a) IFI27, IFI44, IFI44L, IFI6 and RSAD2; or (b) IFI44, IFI44L, IFI6 and RSAD2; or (c) IFI27, IFI44L, IFI6 and RSAD2; or (d) IFI27, IFI44, IFI6 and RSAD2; or (e) IFI27, IFI44, IFI44L, and RSAD2; or (f) IFI27, IFI44, IFI44L, and IFI6.

The IFNα-inducible PD markers in an expression profile may consist of (a) IFI27, IFI44, IFI44L, IFI6 and RSAD2; or (b) IFI44, IFI44L, IFI6 and RSAD2; or (c) IFI27, IFI44L, IFI6 and RSAD2; or (d) IFI27, IFI44, IFI6 and RSAD2; or (e) IFI27, IFI44, IFI44L, and RSAD2; or (f) IFI27, IFI44, IFI44L, and IFI6.

B. Serum Proteins

The IFNα-inducible PD markers in an expression profile may include alterations in any one or more of serum protein levels of adiponectin, alpha-fetoprotein, apolipoprotein CHI, beta-2 microglobulin, cancer antigen 125, cancer antigen 19-9, eotaxin, FABP, factor VII, ferritin, IL-10, IL-12p70, IL-16, IL-18, IL-1ra, IL-3, MCP-1, MMP-3, myoglobin, SGOT, tissue factor, TIMP-1, TNF RII, TNF-alpha, VCAM-1, vWF, BDNK, complement 3, CD40 ligand, EGF, ENA-78, EN-RAGE, IGF-1, MDC, myeloperoxidase, RANTES, or thrombopoietin.

The IFNα-inducible PD markers in an expression profile may include alterations in any one or more of serum protein levels of adiponectin, alpha-fetoprotein, apolipoprotein CHI, beta-2 microglobulin, cancer antigen 125, cancer antigen 19-9, eotaxin, FABP, factor VII, ferritin, IL-10, IL-12p70, IL-16, IL-18, IL-1ra, IL-3, MCP-1, MMP-3, myoglobin, SGOT, tissue factor, TIMP-1, TNF RII, TNF-alpha, VCAM-1, or vWF.

The IFNα-inducible PD markers in an expression profile may include alterations in any one or more of serum protein levels of BDNK, complement 3, CD40 ligand, EGF, ENA-78, EN-RAGE, IGF-1, MDC, myeloperoxidase, RANTES, or thrombopoietin.

IFNα-inducible PD marker expression profiles may include up-regulated expression or activity of genes in cells exposed to elevated IFNα levels relative to baseline. Up-regulated expression or activity of genes includes an increase in expression of mRNA from a gene, an increase in expression of a protein encoded by a gene, or an increase in activity of a protein encoded by a gene. The expression or activity of the genes may be up-regulated as a direct or indirect response to IFNα.

C. Interferon Subtypes

The patient comprising the type I IFN or IFNα-inducible PD marker expression profile may further comprise upregulation of expression of any number of IFNα or type-I IFN subtypes. The IFNα or type-I IFN subtypes may include any more than one, more than two, more than three, more than four, more than five, more than six, more than seven, more than eight, more than nine, or more than ten IFNα or type-I IFN subtypes. These subtypes may include IFNα1, IFNα2, IFNα4, IFNα5, IFNα6, IFNα7, IFNα8, IFNα10, IFNα14, IFNα17, IFNα21, IFNβ, or IFNω. The patient may comprise upregulation of expression of IFN subtypes IFNα1, IFNα2, IFNα8, and IFNα14.

Alternatively, a patient treated in the methods encompassed by the disclosure may simply be one identified as comprising a gene expression profile with upregulation of expression of any number of IFNα or type-I IFN subtypes. The IFNα or type-I IFN subtypes may include any more than one, more than two, more than three, more than four, more than five, more than six, more than seven, more than eight, more than nine, or more than ten IFNα or type-I IFN subtypes. These subtypes may include IFNα1, IFNα2, IFNα4, IFNα5, IFNα6, IFNα7, IFNα8, IFNα10, IFNα14, IFNα17, IFNα21, IFNβ, or IFNω. These subtypes may include IFNα1, IFNα2, IFNα8, and IFNα14.

D. IFN-α Receptors

The patient comprising the type I IFN or IFNα-inducible PD marker expression profile may further comprise upregulation of expression of IFNα receptors, either IFNAR1 or IFNAR2, or both, or TNFα, or IFNγ, or IFNγ receptors (either IFNGR1, IFNGR2, or both IFNGR1 and IFNGR2). The patient may simply be identified as one who comprises upregulation of expression of IFNα receptors, either IFNAR1 or IFNAR2, or both, or TNFα, or IFNγ, or IFNγ receptors (either IFNGR1, IFNGR2, or both IFNGR1 and IFNGR2).

### II. Upregulation

The upregulation or downregulation of the type I IFN or IFNα-inducible PD markers in the patient's expression profile may be by any degree relative to that of a sample from a control (which may be from a sample that is not disease tissue of the patient (e.g., non-lesional skin of a psoriasis patient) or from a healthy person not afflicted with the disease or disorder) or may be relative to that of genes from the patient whose expression is not changed by the disease (so called “house keeping” genes.)

The degree upregulation or downregulation may be at least 10%, at least 15%, at least 20%, at least 25%, at least 30%, at least 40%, at least 50%, at least 60%, at least 70%, at least 75%, at least 80%, at least 85, at least 90%, at least 95%, at least 100%, at least 125%, at least 150%, or at least 200%, or at least 300%, or at least 400%, or at least 500% or more that of the control or control sample.

Type I IFN or IFNα-inducible PD marker expression profile may be calculated as the average fold increase in the expression or activity of the set of genes comprised by the PD marker. The Type I IFN or IFNα-inducible PD marker expression profile may also be calculated as the difference between the mean Ct (cycle threshold) for the four target genes and the mean Ct of three control genes.

The average fold increase in the expression or activity of the set of genes may be between at least about 2 and at least about 15, between at least about 2 and at least about 10, or between at least about 2 and at least about 5. The average fold increase in the expression or activity of the set of genes may be at least about 2, at least about 2.5, at least about 3, at least about 3.5, at least about 4, at least about 4.5, at least about 5, at least about 5.5, at least about 6, at least about 6.5, at least about 7, at least about 8, at least about 9 or at least about 10.

The degree of increased expression permits the identification of a fold change cutoff for identifying signature positive and signature negative patients suffering from autoimmune diseases. In one embodiment, the cutoff is at least about 2. In another embodiment, the cutoff is at least about 2.5. In another embodiment, the cutoff is at least about 3. In another embodiment, the cutoff is at least about 3.5. In another embodiment, the cutoff is at least about 4. In another embodiment, the cutoff is at least about 4.5. In another embodiment, the cutoff is chosen from at least 3.5, 3.6, 3.7, 3.8, 3.9, 4.0, 4.1, 4.2, 4.3, 4.4, and 4.5. In another embodiment the cutoff is between about 2 and about 8. In one embodiment, the cutoff is the mean of the increased expression levels of at least four of IFI27, IFI44, IFI44L, IFI6 and RSAD2. In another embodiment, the cutoff is the median of the increased expression levels of at least four of IFI27, IFI44, IFI44L, IFI6 and RSAD2.

The degree of increased expression also permits the identification of a delta Ct cutoff for identifying signature positive and signature negative patients suffering from autoimmune diseases. In one embodiment, the cutoff is at least about 7.6. In another embodiment, the cutoff is 7.56. The fold change cutoff may be used to determine an appropriate delta Ct cutoff (e.g., 1<log 2 of the fold change <3 corresponds to delta Ct range of 8.65 to 6.56.). Thus, in another embodiment, the delta Ct cutoff is between about 6.56 to about 8.56.

Furthermore, the patient may overexpress or have a tissue that overexpresses a type I IFN subtype at least 10%, at least 15%, at least 20%, at least 25%, at least 30%, at least 40%, at least 50%, at least 60%, at least 70%, at least 75%, at least 80%, at least 90%, at least 100%, at least 125%, at least 150%, or at least 200%, or at least 300%, or at least 400%, or at least 500% that of the control. The type I IFN subtype may be any one of IFNα1, IFNα2, IFNα4, IFNα5, IFNα6, IFNα7, IFNα8, IFNα10, IFNα14, IFNα17, IFNα21, IFNβ, or IFNω. The type I IFN subtypes may include all of IFNα1, IFNα2, IFNα8, and IFNα14.

The up-regulated expression or activity of any gene detected in a sample, by probes, or by probes in kits in an IFNα-inducible PD marker expression profile may be at least 1.2-fold, at least 1.25-fold, at least 1.3-fold, at least 1.4-fold, at least 1.5-fold, at least 2.0-fold, at least 2.25-fold, at least 2.5-fold, at least 2.75-fold, at least 3.0-fold, at least 3.5-fold, at least 4.0-fold, at least 4.5-fold, at least 5.0-fold, at least 6.0-fold, at least 7.0-fold, at least 8.0-fold, at least 9.0-fold, at least 10.0-fold, at least 15.0-fold, at least 20.0-fold, at least 25.0-fold, or at least 50.0-fold relative to baseline levels of control cells, e.g., cells of healthy volunteers or cells of control animals or cells not exposed to IFNα in culture. All of the genes in the IFNα-inducible PD marker expression profile may have up-regulated expression or activity at the same fold increase. Alternatively, the genes in the PD marker expression profile may have varying levels of up-regulated expression or activity.

A. Measuring Upregulation

Up- or down-regulation of gene expression or activity of IFNα-inducible PD markers may be determined by any means known in the art. For example, up- or down-regulation of gene expression may be detected by determining mRNA levels. mRNA expression may be determined by northern blotting, slot blotting, quantitative reverse transcriptase polymerase chain reaction, or gene chip hybridization techniques. See U.S. Pat. Nos. 5,744,305 and 5,143,854 for examples of making nucleic acid arrays for gene chip hybridization techniques. See Establishing and functional characterization of an HEK-293 cell line expressing autofluorescently tagged-actin (pEYFP-ACTIN) and the neurokinin type 1 receptor (NK1-R) Hrovat, A; Zavec, A B; Pogacnik, A; Frangez, R; Vrecl, M 2010 Cellular & Molecular Biology Letters 1, 55-69, Expression profiles of proliferative and antiapoptotic genes in sporadic and colitis-related mouse colon cancer models Svec, J; Ergang, P; Mandys, V; Kment, M; Pacha, J 2010 International Journal of Experimental Pathology 1, 44-53, and Protein kinase inhibitors emodin and dichloro-ribofuranosylbenzimidazole modulate the cellular accumulation and cytotoxicity of cisplatin in a schedule-dependent manner Kurokawa, T; He, G A; Siddik, Z H 2010 Cancer Chemotherapy and Pharmacology 3, 427-436, for examples of how to use the TAQMAN® method for measuring gene expression.

Primers that selectively bind to targets in polymerase chain reactions (PCR) can be chosen based on empirically determining primers that hybridize in a PCR reaction and produce sufficient signal to detect the target over background, or can be predicted using the melting temperature of the primer:target duplex as described in Maniatis et al. Molecular Cloning, Second Edition, Section 11.46. 1989. Similarly, probes for detecting PCR products in a TAQMAN® or related method can be empirically chosen or predicted. Such primers and probes (collectively “oligonucleotides”) may be between 10 and 30 nucleotides or greater in length.

Up- or down-regulation of gene expression or activity of IFNα-inducible PD markers may be determined by detecting protein levels. Methods for detecting protein expression levels include immuno-based assays such as enzyme-linked immunosorbant assays, western blotting, protein arrays, and silver staining.

An IFNα-inducible PD marker expression profile may comprise a profile of protein activity. Up- or down-regulation of gene expression or activity of IFNα-inducible PD markers may be determined by detecting activity of proteins including, but not limited to, detectable phosphorylation activity, de-phosphorylation activity, or cleavage activity. Furthermore, up- or down-regulation of gene expression or activity of IFNα-inducible PD markers may be determined by detecting any combination of these gene expression levels or activities.

### III. Type I IFN or an IFNα-Inducible Disease, Disorder, or Conditions

A type I IFN or an IFNα-inducible disease, disorder, or condition is any that exhibits a type I IFN or an IFNα PD marker expression profile or gene signature. A PD marker expression profile and a gene signature will be understood to be equivalent. These diseases, disorders, or conditions include those with an autoimmune component such as systemic lupus erythematosus (SLE), insulin dependent diabetes mellitus, inflammatory bowel disease (including Crohn's disease, ulcerative colitis, and Celiac's disease), multiple sclerosis, psoriasis, autoimmune thyroiditis, schleroderma, rheumatoid arthritis, glomerulonephritis, idiopathic inflammatory myositis, Sjogren's syndrome, vasculitis, inclusion body myositis (IBM), dermatomyositis, polymyositis, lupus nephritis, and sarcoidosis. Other diseases, disorders, or conditions include graft versus host disease and transplant rejection.

A. Patient Symptoms

The patients may also exhibit any of a number of symptoms as discussed in, e.g., International Application No. PCT/US2007/024941, or may have a clinical SLEDAI score or BILAG score as discussed in the same. These symptoms may include fatigue, organ damage, malar rash, and alopecia. The patient may be scored using a known clinical scoring system, e.g., SLEDAI which is an index of SLE disease activity as measured and evaluated within the last 10 days (Bombardier C, Gladman D D, Urowitz M B, Caron D, Chang C H and the Committee on Prognosis Studies in SLE: Derivation of the SLEDAI for Lupus Patients. Arthritis Rheum 35:630-640, 1992.). Disease activity under the SLEDAI scoring system can range from 0 to 105. The following categories of SLEDAI activity have been defined: no activity (SLEDAI=0); mild activity (SLEDAI=1-5); moderate activity (SLEDAI=6-10); high activity (SLEDAI=11-19); very high activity (SLEDAI=20 or higher). (Griffiths, et al., Assessment of Patients with Systemic Lupus Erythematosus and the use of Lupus Disease Activity Indices). Another disease scoring index is the BILAG index which is an activity index of SLE that is based on specific clinical manifestations in eight organ systems: general, mucocutaneous, neurological, musculoskeletal, cardiovascular, respiratory, renal, and hematology results. Scoring is based on a letter system, but weighted numerical scores can also be assigned to each letter, making it possible to calculate a BILAG score in the range of 0-72. (Griffiths, et al., Assessment of Patients with Systemic Lupus Erythematosus and the use of Lupus Disease Activity Indices). Other scoring indices include the PGA score, the composite responder index (CRI), and the ANAM4™ test. The methods described herein, e.g., of treating an autoimmune disorder, may be used for any subject identified as having any activity level of disease activity as measured by any classification methodology known in the art, e.g., mild, moderate, high, or very high. The methods described herein, e.g., of treating an autoimmune disorder, may result in a decrease in a patient's symptoms or may result in an improvement in a score of disease for the patient's type I IFN or an IFNα-inducible disease, disorder, or condition.

### IV. Therapeutic Agents

A therapeutic agent may be administered to a patient or a patient may be identified as a candidate for administration of an agent or a therapeutic agent. A therapeutic agent may modulate type 1 interferon or IFNα activity. Suitable therapeutic agents include molecules that bind to and modulate type I IFN or IFNα activity. Suitable therapeutic agents include molecules that bind to and modulate activity of receptors of type I interferons or IFNα. The therapeutic agent may be a small molecule or a biological agent. If the therapeutic agent is a small molecule it may be synthesized or identified and isolated from a natural source.

If the therapeutic agent is a biological agent, it may be an antibody specific for any subtype(s) of type I IFN or IFNα. For instance, the antibody may be specific for any one of IFNα1, IFNα2, IFNα4, IFNα5, IFNα6, IFNα7, IFNα8, IFNα10, IFNα14, IFNα17, IFNα21, IFNβ, or IFNω. Alternatively, the antibody may be specific for any two, any three, any four, any five, any six, any seven, any eight, any nine, any ten, any eleven, any twelve type I IFN of IFNα subtypes. If the antibody is specific for more than one type I IFN subtype, the antibody may be specific for IFNα1, IFNα2, IFNα4, IFNα5, IFNα8, IFNα10, and IFNα21; or it may be specific for IFNα1, IFNα2, IFNα4, IFNα5, IFNα8, and IFNα10; or it may be specific for IFNα1, IFNα2, IFNα4, IFNα5, IFNα8, and IFNα21; or it may be specific for IFNα1, IFNα2, IFNα4, IFNα5, IFNα 10, and IFNα21. Antibodies specific for type I IFN or IFNα include MEDI-545, any biologic or antibody other than MEDI-545, antibodies described in U.S. patent application Ser. No. 11/009,410 filed Dec. 10, 2004 and Ser. No. 11/157,494 filed Jun. 20, 2005, 9F3 and other antibodies described in U.S. Pat. No. 7,087,726 (Example 1 and Example 2, those disclosed in Table 3 and Table 4, and/or those disclosed in the table entitled “Deposit of Material” on lines 25-54, column 56), NK-2 and YOK5/19 (WO 84/03105), LO-22 (U.S. Pat. No. 4,902,618), 144 BS (U.S. Pat. No. 4,885,166), and EBI-1, EBI-2, and EBI-3 (EP 119476). A therapeutic agent that modulates IFNα activity may neutralize IFNα activity. One of skill in the art is well aware of preparation and formulation of such biological agents and methods of their administration.

MEDI-545 is a fully human, 147,000 Dalton IgG1k monoclonal antibody (Mab) that binds to a majority of interferon-alpha (IFN-α) subtypes. MEDI-545 is made from 100% human protein sequences, thereby making it a fully human monoclonal antibody. Fully human monoclonal antibodies may have advantages over other forms of monoclonal antibodies, such as chimeric and humanized antibodies, as they may have a more favorable safety profile and may be eliminated less rapidly from the human body, thereby possibly reducing the frequency of dosing. MEDI-545 was derived from an IgG4 antibody, 13H5, which was selected based on functional assays as having the most desirable properties for a potential therapeutic agent. 13H5 was subsequently converted to an IgG1 antibody isotype, produced in CHO cells, and selected for further characterization and preclinical development with an initial designation of MDX-1103, now referred to as MEDI-545. See also U.S. Patent Application Publication No. 2007/0014724; PCT Application PCT/US2008/058133 filed Mar. 25, 2008 entitled “Antibodies with Decreased Deamidation Profiles,” and PCT Application PCT/US2008/058132 filed Mar. 25, 2008, each of which is hereby incorporated by reference in their entirety for all purposes.

The therapeutic agent may be an antibody against an interferon receptor, including those disclosed in U.S. Pat. Nos. 7,619,070 and 7,662,381 and International Application No. PCT/US2009/033358.

A. Antibodies

The antibody may be a synthetic antibody, a monoclonal antibody, polyclonal antibodies, a recombinantly produced antibody, an intrabody, a multispecific antibody (including bi-specific antibodies), a human antibody, a humanized antibody, a chimeric antibody, a single-chain Fv (scFv) (including bi-specific scFv), a BiTE molecule, a single chain antibody, a Fab fragments, a F(ab′) fragment, a disulfide-linked Fv (sdFv), or an epitope-binding fragment of any of the above. The antibody may be any of an immunoglobulin molecule or immunologically active portion of an immunoglobulin molecule. Furthermore, the antibody may be of any isotype. For example, it may be any of isotypes IgG1, IgG2, IgG3 or IgG4. The antibody may be a full-length antibody comprising variable and constant regions, or an antigen-binding fragment thereof, such as a single chain antibody, or a Fab or Fab′2 fragment. The antibody may also be conjugated or linked to a therapeutic agent, such as a cytotoxin or a radioactive isotope.

In the methods of treatment a second agent other than an agent that binds to modulates IFNα activity, or an agent that binds to and modulates the activity of a receptor of a type I interferon or IFNα may be administered to the patient. Second agents include, but are not limited to, non-steroidal anti-inflammatory drugs such as ibuprofen, naproxen, sulindac, diclofenac, piroxicam, ketoprofen, diflunisal, nabumetone, etodolac, and oxaprozin, indomethacin; anti-malarial drugs such as hydroxychloroquine; corticosteroid hormones, such as prednisone, hydrocortisone, methylprednisolone, and dexamethasone; methotrexate; immunosuppressive agents, such as azathioprine and cyclophosphamide; and biologic agents that, e.g., target T cells such as Alefacept and Efalizumab, or target TNFα, such as, Enbrel, Remicade, and Humira.

B. Identifying Candidate Therapeutic Agents

A candidate therapeutic for treating IFNα-mediated disorders may be identified by the methods encompassed by the disclosure. Candidate therapeutics may be any type of molecule including a small molecule or a biological agent. A candidate therapeutic identified by the methods encompassed by the disclosure may immediately be identified as useful as a therapeutic for a disease, disorder, or condition. Alternatively, a candidate therapeutic identified by the methods encompassed by the disclosure may need to be further tested and/or modified before selection for treating patients. Alternatively, a candidate therapeutic identified by the methods encompassed by the disclosure may, after further testing, be de-selected as a molecule for treating patients.

In methods that identify candidate therapeutics, cells comprising an IFNα-inducible PD marker expression profile are contacted with an agent. The cells may be any type of cells, such as commercially available immortalized cell lines that comprise an IFNα-inducible PD marker expression profile, commercially available immortalized cell lines that have been treated with IFNα to induce an IFNα-inducible PD marker expression profile, cells isolated from a patient having an IFNα-inducible PD marker expression profile, or cells isolated from a healthy patient and treated with IFNα to induce an IFNα-inducible PD marker expression profile.

Presence or absence of a change in the IFNα-inducible PD marker expression profile of the cells is detected following contacting the cells with the agent. Presence of change may be any change in IFNα-inducible PD marker expression profile including at least a 10% decrease in up-regulated expression or activity of at least 1 gene in the IFNα-inducible PD marker expression profile, at least a 20% decrease of the at least 1 up-regulated gene, at least a 30% decrease of the at least up-regulated 1 gene, at least a 40% decrease of the at least 1 up-regulated gene, at least a 50% decrease of the at least 1 up-regulated gene, at least a 60% decrease of the at least 1 up-regulated gene, at least a 70% decrease of the at least 1 up-regulated gene, at least a 75% decrease of the at least 1 up-regulated gene, at least an 80% decrease of the at least 1 up-regulated gene, at least an 85% decrease of the at least 1 up-regulated gene, at least a 90% decrease of the at least 1 up-regulated gene, at least a 95% decrease of the at least 1 up-regulated gene, at least a 96% decrease of the at least 1 up-regulated gene, at least a 97% decrease of the at least 1 up-regulated gene, at least a 98% decrease of the at least 1 up-regulated gene, at least a 99% decrease of the at least 1 up-regulated gene, or a 100% decrease of the at least 1 up-regulated gene.

### V. Neutralization of the Type I IFN or IFNα-Inducible Profile in Patients

Treatment with the agent may result in neutralization of the type I IFN or IFNα-inducible profile. Treatment with the agent may result in a decrease in one or more symptoms of the type I IFN or an IFNα-mediated disease or disorder. Treatment with the agent may result in fewer flare-ups related to the type I IFN or an IFNα-mediated disease or disorder. Treatment with the agent may result in improved prognosis for the patient having the type I IFN or an IFNα-mediated disease or disorder. Treatment with the agent may result in a higher quality of life for the patient. Treatment with the agent may alleviate the need to co-administer second agents or may lessen the dosage of administration of the second agent to the patient. Treatment with the agent may reduce the number of hospitalizations of the patient that are related to the type I IFN or an IFNα-mediated disease or disorder.

The agent that binds to and modulates type I IFN or IFNα activity may neutralize a type I IFN or IFNα-inducible profile. Neutralization of the type I IFN or IFNα-inducible profile may be a reduction in at least one, at least two, at least three, at least four genes. Neutralization of the type I IFN or IFNα-inducible profile is a reduction of at least 2%, at least 3%, at least 4%, at least 5%, at least 7%, at least 8%, at least 10%, at least 15%, at least 25%, at least 30%, at least 35%, at least 40%, at least 45%, at least 50%, at least 60%, at least 70%, at least 75%, at least 80%, or at least 90% of any of the at least one, at least two, at least three, at least four genes up-regulated in the type I IFN or IFNα-inducible profile. Alternatively, neutralization of the type I IFN or IFNα-inducible profile refers to a reduction of expression of up-regulated type I IFN or IFNα-inducible genes that is within at most 50%, at most 45%, at most 40%, at most 35%, at most 30%, at most 25%, at most 20%, at most 15%, at most 10%, at most 5%, at most 4%, at most 3%, at most 2%, or at most 1% of expression levels of those type I IFN or IFNα-inducible genes in a control sample. If the agent that binds to and modulates type I IFN or IFNα activity is a biologic agent, such as an antibody, the agent may neutralize the type I IFN or IFNα profile at doses of 0.3 to 30 mg/kg, 0.3 to 10 mg/kg, 0.3 to 3 mg/kg, 0.3 to 1 mg/kg, 1 to 30 mg/kg, 3 to 30 mg/kg, 5 to 30 mg/kg, 10 to 30 mg/kg, 1 to 10 mg/kg, 3 to 10 mg/kg, or 1 to 5 mg/kg.

The agent that binds to and modulates type I IFN or IFNα activity may further or alternatively neutralize expression of one or more type I IFN or IFNα subtypes. The IFNα or type-I IFN subtypes may include any more than one, more than two, more than three, more than four, more than five, more than six, more than seven, more than eight, more than nine, or more than ten IFNα or type-I IFN subtypes. These subtypes may include IFNα1, IFNα2, IFNα4, IFNα5, IFNα6, IFNα7, IFNα8, IFNα10, IFNα14, IFNα17, IFNα21, IFNβ, or IFNω. These subtypes may include all of IFNα1, IFNα2, IFNα8, and IFNα14. Alternatively, these subtypes may include IFNα1, IFNα2, IFNα4, IFNα5, IFNα8, IFNα10, IFNα21. Neutralization of the IFNα or type-I IFN subtypes may be a reduction of at least 2%, at least 3%, at least 4%, at least 5%, at least 7%, at least 8%, at least 10%, at least 15%, at least 25%, at least 30%, at least 35%, at least 40%, at least 45%, at least 50%, at least 60%, at least 70%, at least 75%, at least 80%, or at least 90% of any of the at least one, at least two, at least three, at least five, at least seven, at least eight, or at least ten of the subtypes. Neutralization of the IFNα or type-I IFN subtypes may be a reduction in expression of IFNα or type-I IFN subtype genes that is within at most 50%, at most 45%, at most 40%, at most 35%, at most 30%, at most 25%, at most 20%, at most 15%, at most 10%, at most 5%, at most 4%, at most 3%, at most 2%, or at most 1% of expression levels of those IFNα or type I IFN subtypes in a control sample. If the agent that binds to and modulates IFNα activity or type I IFN activity is a biologic agent, such as an antibody, the agent may neutralize the IFNα or type I IFN subtypes at doses of 0.3 to 30 mg/kg, 0.3 to 10 mg/kg, 0.3 to 3 mg/kg, 0.3 to 1 mg/kg, 1 to 30 mg/kg, 3 to 30 mg/kg, 5 to 30 mg/kg, 10 to 30 mg/kg, 1 to 10 mg/kg, 3 to 10 mg/kg, or 1 to 5 mg/kg.

The agent that binds to and modulates type I IFN or IFNα activity may further or alternatively neutralize expression of IFNα receptors, either IFNAR1 or IFNAR2, or both, or TNFα, or IFNγ, or IFNγ receptors (either IFNGR1, IFNGR2, or both IFNGR1 and IFNGR2). Neutralization of expression of IFNα receptors, either IFNAR1 or IFNAR2, or both, or TNFα, or IFNγ, or IFNγ receptors (either IFNGR1, IFNGR2, or both IFNGR1 and IFNGR2) may be a reduction of at least 2%, at least 3%, at least 4%, at least 5%, at least 7%, at least 8%, at least 10%, at least 15%, at least 25%, at least 30%, at least 35%, at least 40%, at least 45%, at least 50%, at least 60%, at least 70%, at least 75%, at least 80%, or at least 90% of any of the at least one, at least two, at least three, at least five, or at least six of these genes. Neutralization of expression of IFNα receptors, either IFNAR1 or IFNAR2, or TNFα, or IFNγ, or IFNγ receptors (either IFNGR1, IFNGR2, or both IFNGR1 and IFNGR2) is a reduction of expression of at most 50%, at most 45%, at most 40%, at most 35%, at most 30%, at most 25%, at most 20%, at most 15%, at most 10%, at most 5%, at most 4%, at most 3%, at most 2%, or at most 1% of expression levels of these genes in a control sample. If the agent that binds to and modulates type I IFN or IFNα activity is a biologic agent, such as an antibody, the agent may neutralize expression of IFNα receptors IFNAR1 or IFNAR2, or TNFα, or IFNγ, or IFNγ receptors IFNGR1 or IFNGR2 at doses of 0.3 to 30 mg/kg, 0.3 to 10 mg/kg, 0.3 to 3 mg/kg, 0.3 to 1 mg/kg, 1 to 30 mg/kg, 3 to 30 mg/kg, 5 to 30 mg/kg, 10 to 30 mg/kg, 1 to 10 mg/kg, 3 to 10 mg/kg, or 1 to 5 mg/kg.

C. Patient Samples

Samples may also be obtained from patients in the methods of the disclosure. Samples include any biological fluid or tissue, such as whole blood, saliva, urine, synovial fluid, bone marrow, cerebrospinal fluid, nasal secretions, sputum, amniotic fluid, bronchoalveolar lavage fluid, peripheral blood mononuclear cells, total white blood cells, lymph node cells, spleen cells, tonsil cells, or skin. The samples may be obtained by any means known in the art.

### VI. Methods of Monitoring Disease Progression

In methods of monitoring disease progression of a patient samples from the patient may be obtained before and after administration of an agent, e.g., an agent that binds to and modulates type I IFN or IFNα activity, or an agent that binds to and does not modulate type I IFN or IFNα activity, or a combination of agents that may or may not include an agent that binds to and modulates type I IFN or IFNα activity. Type I IFN or IFNα inducible PD marker expression profiles are obtained in the (before and after agent administration) samples. The type I IFN or IFNα inducible PD marker expression profiles in the samples are compared. Comparison may be of the number of type I IFN or IFNα inducible PD markers present in the samples or may be of the quantity of type I IFN or IFNα inducible PD markers present in the samples, or any combination thereof. Variance indicating efficacy of the therapeutic agent may be indicated if the number or level (or any combination thereof) of up-regulated type I IFN or IFNα inducible PD markers decreases in the sample obtained after administration of the therapeutic agent relative to the sample obtained before administration of the therapeutic agent. The number of up-regulated type I IFN or IFNα inducible PD markers may decrease by at least 1, at least 2, at least 3, at least 4, at least 5, at least 6, at least 7, at least 8, at least 9, or at least 10 fold. The level of any given up-regulated type I IFN or IFNα inducible PD marker may decrease by at least 10%, at least 20%, at least 25%, at least 30%, at least 35%, at least 40%, at least 50%, at least 60%, at least 70%, at least 80%, at least 90%, or at least 95%. The number of up-regulated type I IFN or IFNα inducible PD markers with decreased levels may be at least 1, at least 2, at least 3, or at least 4. Any combination of decreased number and decreased level of up-regulated type I IFN or IFNα inducible PD markers may indicate efficacy. Variance indicating efficacy of the therapeutic agent may be indicated if the number or level (or any combination thereof) of down-regulated type I IFN or IFNα inducible PD markers decreases in the sample obtained after administration of the therapeutic agent relative to the sample obtained before administration of the therapeutic agent.

The sample obtained from the patient may be obtained prior to a first administration of the agent, i.e., the patient is naïve to the agent. Alternatively, the sample obtained from the patient may occur after administration of the agent in the course of treatment. For example, the agent may have been administered prior to the initiation of the monitoring protocol. Following administration of the agent an additional sample may be obtained from the patient and type I IFN or IFNα inducible PD markers in the samples are compared. The samples may be of the same or different type, e.g., each sample obtained may be a blood sample, or each sample obtained may be a serum sample. The type I IFN or IFNα inducible PD markers detected in each sample may be the same, may overlap substantially, or may be similar.

The samples may be obtained at any time before and after the administration of the therapeutic agent. The sample obtained after administration of the therapeutic agent may be obtained at least 2, at least 3, at least 4, at least 5, at least 6, at least 7, at least 8, at least 9, at least 10, at least 12, or at least 14 days after administration of the therapeutic agent. The sample obtained after administration of the therapeutic agent may be obtained at least 2, at least 3, at least 4, at least 5, at least 6, at least 7, or at least 8 weeks after administration of the therapeutic agent. The sample obtained after administration of the therapeutic agent may be obtained at least 2, at least 3, at least 4, at least 5, or at least 6 months following administration of the therapeutic agent.

Additional samples may be obtained from the patient following administration of the therapeutic agent. At least 2, at least 3, at least 4, at least 5, at least 6, at least 7, at least 8, at least 9, at least 10, at least 12, at least 15, at least 20, at least 25 samples may be obtained from the patient to monitor progression or regression of the disease or disorder over time. Disease progression may be monitored over a time period of at least 1 week, at least 2 weeks, at least 3 weeks, at least 4 weeks, at least 5 weeks, at least 6 weeks, at least 7 weeks, at least 2 months, at least 3 months, at least 4 months, at least 5 months, at least 6 months, at least 1 year, at least 2 years, at least 3 years, at least 4 years, at least 5 years, at least 10 years, or over the lifetime of the patient. Additional samples may be obtained from the patient at regular intervals such as at monthly, bi-monthly, once a quarter year, twice a year, or yearly intervals. The samples may be obtained from the patient following administration of the agent at regular intervals. For instance, the samples may be obtained from the patient at one week following each administration of the agent, or at two weeks following each administration of the agent, or at three weeks following each administration of the agent, or at one month following each administration of the agent, or at two months following each administration of the agent. Alternatively, multiple samples may be obtained from the patient following each administration of the agent.

Disease progression in a patient may similarly be monitored in the absence of administration of an agent. Samples may periodically be obtained from the patient having the disease or disorder. Disease progression may be identified if the number of type I IFN or IFNα inducible PD markers increases in a later-obtained sample relative to an earlier obtained sample. The number of type I IFN or IFNα inducible PD markers may increase by at least 1, at least 2, at least 3, at least 4, at least 5, at least 6, at least 7, at least 8, at least 9, or at least 10. Disease progression may be identified if level of any given up-regulated type I IFN or IFNα inducible PD marker increases by at least 10%, at least 20%, at least 25%, at least 30%, at least 35%, at least 40%, at least 50%, at least 60%, at least 70%, at least 80%, at least 90%, or at least 95%. Disease progression may be identified if level of any given down-regulated type I IFN or IFNα inducible PD marker decreases by at least 10%, at least 20%, at least 25%, at least 30%, at least 35%, at least 40%, at least 50%, at least 60%, at least 70%, at least 80%, at least 90%, or at least 95%. The number of up-regulated type I IFN or IFNα inducible PD markers with increased levels may be at least 1, at least 2, at least 3, at least 4, at least 5, at least 6, at least 7, at least 8, at least 9, at least 10, at least 15, at least 20, at least 25, at least 30, or at least 35. The number of down-regulated type I IFN or IFNα inducible PD markers with decreased levels may be at least 1, at least 2, at least 3, at least 4, at least 5, at least 6, at least 7, at least 8, at least 9, at least 10, at least 15, at least 20, at least 25, at least 30, or at least 35. Any combination of increased number and increased level of up-regulated type I IFN or IFNα inducible PD marker may indicate disease progression. Alternatively, or in combination, any combination of decreased number and decreased level of down-regulated type I IFN or IFNα inducible PD marker may indicate disease progression. Disease regression may also be identified in a patient having a disease or disorder, not treated by an agent. In this instance, regression may be identified if the number of type I IFN or IFNα inducible PD markers decreases in a later-obtained sample relative to an earlier obtained sample. The number of type I IFN or IFNα inducible PD markers may decrease by at least 1, at least 2, at least 3, at least 4, at least 5, at least 6, at least 7, at least 8, at least 9, or at least 10. Disease regression may be identified if level of any given up-regulated type I IFN or IFNα inducible PD marker decreases by at least 10%, at least 20%, at least 25%, at least 30%, at least 35%, at least 40%, at least 50%, at least 60%, at least 70%, at least 80%, at least 90%, or at least 95%. Disease regression may be identified if level of any given down-regulated type I IFN or IFNα inducible PD marker increases by at least 10%, at least 20%, at least 25%, at least 30%, at least 35%, at least 40%, at least 50%, at least 60%, at least 70%, at least 80%, at least 90%, or at least 95%. The number of up-regulated type I IFN or IFNα inducible PD markers with decreased levels may be at least 1, at least 2, at least 3, at least 4, at least 5, at least 6, at least 7, at least 8, at least 9, at least 10, at least 15, at least 20, at least 25, at least 30, or at least 35. The number of down-regulated type I IFN or IFNα inducible PD markers with increased levels may be at least 1, at least 2, at least 3, at least 4, at least 5, at least 6, at least 7, at least 8, at least 9, at least 10, at least 15, at least 20, at least 25, at least 30, or at least 35. Disease progression or disease regression may be monitored by obtaining samples over any period of time and at any interval. Disease progression or disease regression may be monitored by obtaining samples over the course of at least 1 week, at least 2 weeks, at least 3 weeks, at least 4 weeks, at least 5 weeks, at least 6 weeks, at least 7 weeks, at least 2 months, at least 3 months, at least 4 months, at least 5 months, at least 6 months, at least 1 year, at least 2 years, at least 3 years, at least 4 years, at least 5 years, at least 10 years, or over the lifetime of the patient. Disease progression or disease regression may be monitored by obtaining samples at least monthly, bi-monthly, once a quarter year, twice a year, or yearly. The samples need not be obtained at strict intervals.

### VII. Kits and Probes

The disclosure also encompasses kits and probes. The probes may be any molecule that detects any expression or activity of any gene that may be included in an IFNα-inducible PD marker expression profile.

### VIII. Methods of Detecting IFN Activity

The disclosure also encompasses methods of detecting IFN activity. These methods may employ cells comprising a polynucleotide sequence comprising a reporter gene under the control of an interferon-stimulated response element. The cells comprising the polynucleotide sequence may be any cells amenable to transfection or transformation with a polynucleotide sequence and that can be maintained in culture. These cells include animal cells, bacterial cells, yeast cells, insect cells, or plant cells. These cells may be adherent or may grow in suspension. If the cells are animal cells, they may be from a known cell line such as HeLa, COS, NIH3T3, AGS, 293, CHO, Huh-7, HUVEC, MCF-7, C6, BHK-21, BNL CL 2, C2C12, HepG2, and ATDC5. Countless other cell lines are known and can be obtained by those of skill in the art. The cells may alternatively be primary cells that have or have not been immortalized.

The cells may comprise a polynucleotide sequence comprising a reporter gene under the control of an interferon-stimulated response element. The polynucleotide sequence may be stably integrated in the DNA of the cell or may be an extrachomosomal element that is stably or transiently in the cell. The polynucleotide may have been introduced to the cell as a naked polynucleotide molecule, a polynucleotide molecule complexed with lipids or other molecules, or a polynucleotide in a virus particle.

If the polynucleotide was introduced as a naked polynucleotide molecule, the polynucleotide may have been a linear or a circular molecule. Non-limiting examples of circular polynucleotide molecules include plasmids, and artificial chromosomes. These vectors may be cleaved with enzymes, for example, to generate linear polynucleotide molecules.

Furthermore, if the polynucleotide was introduced as a naked polynucleotide it may have been introduced into the cells by any of many well known techniques in the art. These techniques include, but are not limited to, electroporation, microinjection, and biolistic particle delivery. See, also, e.g., Loeffler and Behr, 1993, Meth. Enzymol. 217:599-618; Cohen et al., 1993, Meth. Enzymol. 217:618-644; Clin. Pharma. Ther. 29:69-92 (1985), Sambrook, et al. Molecular Cloning: A Laboratory Manual. 2nd, ed., Cold Spring Harbor Laboratory, Cold Spring Harbor Laboratory Press, Cold Spring Harbor, N. Y., 1989 and Ausubel et al., ed. Current Protocols in Molecular Biology, John Wiley & Sons, Inc., N.Y., N.Y. (1987-2001).

If the polynucleotide was introduced as a complex with lipids or liposomes, it too may have been introduced by one of many known techniques to the skilled artisan. Lipids or liposomes comprise a mixture of fat particles or lipids which bind to DNA or RNA to provide a hydrophobic coated delivery vehicle. Suitable liposomes may comprise any of the conventional synthetic or natural phospholipid liposome materials including phospholipids from natural sources such as egg, plant or animal sources such as phosphatidylcholine, phosphatidylethanolamine, phosphatidylglycerol, sphingomyelin, phosphatidylserine or phosphatidylinositol. Synthetic phospholipids also may be used, e.g., dimyristoylphosphatidylcholine, dioleoylphosphatidylcholine, dioleoylphosphatidycholine and corresponding synthetic phosphatidylethanolamines and phosphatidylglycerols. Lipids or liposomes that may be conjugated with the vector are also commercially available to the skilled artisan. Examples of commercially available lipid or liposome transfection reagents known to those of skill in the art include LIPOFECTAMINE™ (Invitrogen), GENEJUICE® (Novagen), GENEJAMMER® (Stratagene), FUGENE® HD (Roche), MEGAFECTIN™ (Qbiogene), SUPERFECT® (Qiagen), and EFFECTENE® (Qiagen).

If the polynucleotide was introduced as a complex with other molecules it may have been compacted or in a nanosphere. Compacted polynucleotide complexes are described in U.S. Pat. Nos. 5,972,901, 6,008,336, and 6,077,835. Nanospheres are described in U.S. Pat. Nos. 5,718,905 and 6,207,195. These compacted polynucleotide complexes and nanospheres that complex nucleic acids utilize polymeric cations. Typical polymeric cations include gelatin, poly-L-lysine, and chitosan. Alternatively, the polynucleotide may have been complexed with DEAE-dextran, or transfected using techniques such as calcium phosphate coprecipitation, or calcium chloride coprecipitation.

If the polynucleotide was introduced associated with a virus, the virus may have been any well known suitable virus for polynucleotide delivery. Example viruses that may be used as vectors include adenovirus, adeno-associated virus, lentivirus, retrovirus, herpes virus (e.g. herpes simplex virus), vaccina virus, papovirus, Sendai virus, SV40 virus, respiratory syncytial virus, etc.

The polynucleotide sequence may include a reporter gene and an interferon-stimulated response element. The reporter gene may be any one of luciferase, chloramphenicol acetyl transferase, β-galactosidase, green fluorescent protein, β-glucuronidase, or secreted placental alkaline phosphatase. Variations of many of these reporter genes, e.g., green fluorescent protein and luceriferase, are known and can be readily identified and/or produced by those of skill in the art. Other reporter genes in addition to those listed will also be known to those of skill in the art and are readily available. Interferon-stimulated response elements are also known to those of skill in the art. They may be obtained from commercial vendors such as Stratagene, Clonetech, and Biomyx. They have also been reported in, for instance, Alcantara et al. (Nuc. Acid. Res. 30 (2002):2068-2075 and Kirchhoff et al. (Oncogene 18 (1999):3725-3736).

The cells employed in the assay may be incubated with a sample. The sample may be obtained from a patient, from a vendor with patient samples, or a control sample used for calibration or as a control. If the sample is obtained from a patient it may be any biological fluid or tissue, such as whole blood, saliva, urine, synovial fluid, bone marrow, cerebrospinal fluid, nasal secretions, sputum, amniotic fluid, bronchoalveolar lavage fluid, peripheral blood mononuclear cells, total white blood cells, lymph node cells, spleen cells, tonsil cells, or skin.

Expression of the reporter gene is detected by any well known means in the art. The expression, even if “0” indicates IFN activity in the sample. One of skill in the art may further quantitate any level of expression of the reporter gene which may then correlate to level of IFN activity in the sample.

Applicants provide a set of non-limiting embodiments to describe some of the aspects of the disclosure.

### EMBODIMENTS

### Embodiment 1

A method of treating a patient having a type I IFN or an IFNα-mediated disease or disorder comprising:

administering an agent that binds to and modulates type I IFN or IFNα activity;

wherein the patient comprises a type I IFN or IFNα-inducible PD marker expression profile;

and wherein the agent neutralizes the type I IFN or IFNα-inducible PD marker expression profile of the patient.

### Embodiment 2

A method of treating an autoimmune disease patient comprising a moderate or strong type I IFN or an IFNα PD marker profile comprising:

administering an agent that binds to and modulates type I IFN or IFNα activity;

wherein the agent neutralizes the type I IFN or IFNα-inducible PD marker expression profile of the patient.

### Embodiment 3

A method of neutralizing a type I IFN or IFNα-inducible PD marker expression profile in a patient having a disease or disorder, comprising:

administering an agent that binds to and modulates type I IFN or IFNα activity to the patient;

wherein the agent neutralizes the type I IFN or IFNα-inducible PD marker expression profile of the patient.

### Embodiment 4

The method of any one of embodiments 1 to 3 further comprising detecting neutralization of the type I IFN or IFNα-inducible PD marker expression profile of the patient.

### Embodiment 5

The method of any one of embodiments 1 to 4 wherein the type I IFN or IFNα-inducible PD marker expression profile comprises transcripts of PD marker genes.

### Embodiment 6

The method of any one of embodiments 1 to 4 wherein the type I IFN or IFNα-inducible PD marker expression profile comprises polypeptides expressed from PD marker genes.

### Embodiment 7

The method of any one of embodiments 1 to 6 wherein the type I IFN or IFNα-inducible PD marker expression profile comprises up-regulated expression or activity of a set of genes chosen from of:

(a) IFI27, IFI44, IFI44L, IFI6, and RSAD2;

(b) IFI44, IFI44L, IFI6, and RSAD2;

(c) IFI27, IFI44L, IFI6, and RSAD2;

(d) IFI27, IFI44, IFI6, and RSAD2;

(e) IFI27, IFI44, IFI44L, and RSAD2; and

(f) IFI27, IFI44, IFI44L, and IFI6.

### Embodiment 8

The method of any one of embodiments 1 to 6 wherein the type I IFN or IFNα-inducible PD marker expression profile consists of up-regulated expression or activity of a set of genes chosen from:

(a) IFI27, IFI44, IFI44L, IFI6, and RSAD2;

(b) IFI44, IFI44L, IFI6, and RSAD2;

(c) IFI27, IFI44L, IFI6, and RSAD2;

(d) IFI27, IFI44, IFI6, and RSAD2;

(e) IFI27, IFI44, IFI44L, and RSAD2; and

(f) IFI27, IFI44, IFI44L, and IFI6.

### Embodiment 9

The method embodiment 7 or 8 wherein the up-regulated expression or activity of a set of genes is calculated as an average fold increase in the expression or activity of the set of genes.

### Embodiment 10

The method embodiment 9 wherein the average fold increase in the expression or activity of the set of genes is between at least about 3 and at least about 15, between at least about 3 and at least about 10, or between at least about 3 and at least about 5.

### Embodiment 11

The method embodiment 9 wherein the average fold increase in the expression or activity of the set of genes is at least about 2, at least about 2.5, at least about 3, at least about 3.5, at least about 4, at least about 4.5, at least about 5, at least about 5.5, at least about 6, at least about 6.5, at least about 7, at least about 8, at least about 9 or at least about 10.

### Embodiment 12

The method of any one of embodiments 1 to 11 wherein the agent is a biologic agent.

### Embodiment 13

The method of embodiment 12 wherein the agent is an antibody.

### Embodiment 14

The method of embodiment 13 wherein the antibody is MEDI-545.

### Embodiment 15

The method of embodiment 13 wherein the antibody is specific for one or more type I IFN or IFNα subtype but is not MEDI-545.

### Embodiment 16

The method of any one of embodiments 1 to 15 wherein the administering the agent alleviates one or more symptoms of the disease or disorder.

### Embodiment 17

The method of any one of embodiments 13 to 16 wherein the antibody is administered at a dose between approximately 0.03 and 30 mg/kg.

### Embodiment 18

The method of embodiment 17 wherein the antibody is administered at a dose between 0.3 and 3 mg/kg or between 0.03 and 1 mg/kg.

### Embodiment 19

The method of any one of embodiments 1 to 18 wherein the agent neutralizes the type I IFN or IFNα-inducible PD marker expression profile of the patient by at least 10%, by at least 20%, by at least 30%, by at least 40% or by at least 50%.

### Embodiment 20

The method of any one of embodiments 1 to 19 wherein the disease or disorder is one of lupus, lupus nephritis, dermatomyositis, polymyositis, psoriasis, SSc, vasculitis, sarcoidosis, Sjogren's syndrome, or idiopathic inflammatory myositis.

### Embodiment 21

The method of embodiment 20 wherein the disease or disorder is lupus.

### Embodiment 22

The method of embodiment 20 wherein the disease or disorder is psoriasis.

### Embodiment 23

The method of any one of embodiments 1 to 22 wherein the type I IFN or IFNα-inducible PD marker expression profile comprises up-regulated expression or activity of at least IFNα subtypes 1, 2, 8, and 14.

### Embodiment 24

A method of monitoring or prognosing autoimmune disease progression of a patient comprising:

obtaining a first IFNα-inducible PD marker expression profile in a first sample from a patient.

### Embodiment 25

The method of embodiment 24 wherein the first IFNα-inducible PD marker expression profile is a strong profile and the patient prognosis is disease progression.

### Embodiment 26

The method of embodiment 25 wherein the autoimmune disease is SLE and the progression is an SLE flare.

### Embodiment 27

The method of embodiment 26 wherein the first IFNα-inducible PD marker expression profile is a weak profile and the patient prognosis is disease regression.

### Embodiment 28

The method of embodiment 24 further comprising:

obtaining a second IFNα-inducible PD marker expression profile in a second sample from a patient;

wherein an increase in number or level of type I IFN or IFNα inducible PD markers in the second relative to the first expression profile prognoses disease progression; or

wherein a decrease in number or level of type I IFN or IFNα inducible PD markers in the second relative to the first expression profile prognoses disease regression.

### Embodiment 29

The method of any one of embodiments 26 to 28 wherein the type I IFN or IFNα-inducible PD marker expression profile comprises transcripts of PD marker genes.

### Embodiment 30

The method of any one of embodiments 24 to 28 wherein the type I IFN or IFNα-inducible PD marker expression profile comprises polypeptides expressed from PD marker genes.

### Embodiment 31

The method of any one of embodiments 24 to 28 wherein the type I IFN or IFNα-inducible PD marker expression profile comprises expression or activity of a set of genes chosen from:

(a) IFI27, IFI44, IFI44L, IFI6, and RSAD2;

(b) IFI44, IFI44L, IFI6, and RSAD2;

(c) IFI27, IFI44L, IFI6, and RSAD2;

(d) IFI27, IFI44, IFI6, and RSAD2;

(e) IFI27, IFI44, IFI44L, and RSAD2; and

(f) IFI27, IFI44, IFI44L, and IFI6.

### Embodiment 32

The method of any one of embodiments 24 to 28 wherein the type I IFN or IFNα-inducible PD marker expression profile consists of expression or activity of a set of genes chosen from:

(a) IFI27, IFI44, IFI44L, IFI6, and RSAD2;

(b) IFI44, IFI44L, IFI6, and RSAD2;

(c) IFI27, IFI44L, IFI6, and RSAD2;

(d) IFI27, IFI44, IFI6, and RSAD2;

(e) IFI27, IFI44, IFI44L, and RSAD2; and

(f) IFI27, IFI44, IFI44L, and IFI6.

### Embodiment 33

A method of monitoring disease progression of a patient receiving treatment with a therapeutic agent that binds to and modulates IFNα activity comprising:

obtaining a first IFNα-inducible PD marker expression profile in a first sample from the patient;

administering a therapeutic agent that binds to and modulates IFNα activity;

obtaining a second IFNα-inducible PD marker expression profile in a second sample from the patient; and

comparing the first and the second IFNα-inducible PD marker expression profiles,

wherein a variance in the first and the second IFNα-inducible PD marker expression profiles indicates a level of efficacy of the therapeutic agent that binds to and modulates IFNα activity.

### Embodiment 34

The method of embodiment 33 wherein the first type I IFN or IFNα-inducible PD marker expression profile comprises up-regulated expression or activity of a set of genes chosen from:

(a) IFI27, IFI44, IFI44L, IFI6, and RSAD2;

(b) IFI44, IFI44L, IFI6, and RSAD2;

(c) IFI27, IFI44L, IFI6, and RSAD2;

(d) IFI27, IFI44, IFI6, and RSAD2;

(e) IFI27, IFI44, IFI44L, and RSAD2; and

(f) IFI27, IFI44, IFI44L, and IFI6.

### Embodiment 35

The method of embodiment 33 wherein the first type I IFN or IFNα-inducible PD marker expression profile consists of up-regulated expression or activity of a set of genes chosen from:

(a) IFI27, IFI44, IFI44L, IFI6, and RSAD2;

(b) IFI44, IFI44L, IFI6, and RSAD2;

(c) IFI27, IFI44L, IFI6, and RSAD2;

(d) IFI27, IFI44, IFI6, and RSAD2;

(e) IFI27, IFI44, IFI44L, and RSAD2; and

(f) IFI27, IFI44, IFI44L, and IFI6.

### Embodiment 36

The method of embodiment 34 or 35 wherein the variance is a decrease in up-regulated expression of activity levels of the genes.

### Embodiment 37

The method of any one of embodiments 33 to 35 wherein the disease or disorder is one of lupus, lupus nephritis, dermatomyositis, polymyositis, psoriasis, SSc, vasculitis, sarcoidosis, Sjogren's syndrome, or idiopathic inflammatory myositis.

### Embodiment 38

The method of embodiment 37 wherein the disease is lupus.

### Embodiment 39

The method of any one of embodiments 33 to 38 wherein the therapeutic agent is a small molecule or a biologic agent.

### Embodiment 40

The method of embodiment 39 wherein the biologic agent is an antibody.

### Embodiment 41

The method of embodiment 40 wherein the antibody is MEDI-545 and/or an antibody specific for one or more type I IFN or IFNα subtype but is not MEDI-545.

### Embodiment 42

The method of any one of embodiments 33 to 41 wherein the first IFNα-inducible PD marker expression profile is obtained prior to administration of the therapeutic agent.

### Embodiment 43

The method of any one of embodiments 33 to 41 wherein the first IFNα-inducible PD marker expression profile is obtained at the time of administration of the therapeutic agent.

### Embodiment 44

The method of any one of embodiments 33 to 43 wherein the first and the second sample are whole blood or serum.

### Embodiment 45

The method of any one of embodiments 33 to 44 further comprising obtaining a third IFNα-inducible PD marker expression profile in a third sample from the patient.

### Embodiment 46

The method of embodiment 45 further comprising obtaining a fourth IFNα-inducible PD marker expression profile in a fourth sample from the patient.

### Embodiment 47

The method of embodiment 46 further comprising obtaining a fifth IFNα-inducible PD marker expression profile in a fifth sample from the patient.

### Embodiment 48

The method of embodiment 47 further comprising obtaining a sixth IFNα-inducible PD marker expression profile in a sixth sample from the patient.

### Embodiment 49

The method of any one of embodiments 33 to 48 wherein the second sample is obtained at least one week, at least two weeks, at least three weeks, at least one month or at least two months following administration of the therapeutic agent.

### Embodiment 50

The method of embodiment 45 wherein the third sample is obtained at least 2 days, at least 5 days, at least one week, at least 2 weeks, at least three weeks, at least one month or at least two months following obtaining the second sample.

### Embodiment 51

The method of embodiment 46 wherein the fourth sample is obtained at least two days, at least five days, at least one week, at least two weeks, at least three weeks, at least one month or at least two months following obtaining the third sample.

### Embodiment 52

The method of embodiment 47 wherein the fifth sample is obtained at least two days, at least five days, at least one week, at least two weeks, at least three weeks, at least one month or at least two months following obtaining the fourth sample.

### Embodiment 53

The method of any one of embodiments 33 to 52 wherein variance is a decrease in up-regulated expression or activity of the gene.

### Embodiment 54

The method of embodiment 53 wherein the decrease is at least 10%, at least 20%, at least 25%, at least 30%, at least 40%, at least 45%, at least 50%, at least 60%, at least 70%, at least 75%, at least 80%, at least 85%, at least 90%, at least 95%, at least 96%, at least 97%, at least 98%, at least 99%, or 100%.

### Embodiment 55

A method of identifying a patient as a candidate for a therapeutic agent that binds to and modulates IFNα activity comprising:

detecting presence or absence of an IFNα-inducible PD marker expression profile in a sample from the patient,

wherein detecting presence of the IFNα-induced PD marker expression profile identifies the patient as a candidate for the therapeutic agent that binds to and modulates IFNα activity.

### Embodiment 56

The method of embodiment 55 wherein the IFNα-inducible PD marker expression profile comprises up-regulated expression or activity of a set of genes chosen from:

(a) IFI27, IFI44, IFI44L, IFI6, and RSAD2;

(b) IFI44, IFI44L, IFI6, and RSAD2;

(c) IFI27, IFI44L, IFI6, and RSAD2;

(d) IFI27, IFI44, IFI6, and RSAD2;

(e) IFI27, IFI44, IFI44L, and RSAD2; and

(f) IFI27, IFI44, IFI44L, and IFI6.

### Embodiment 57

The method of embodiment 55 wherein the IFNα-inducible PD marker expression profile consists of up-regulated expression or activity of a set of genes chosen from:

(a) IFI27, IFI44, IFI44L, IFI6, and RSAD2;

(b) IFI44, IFI44L, IFI6, and RSAD2;

(c) IFI27, IFI44L, IFI6, and RSAD2;

(d) IFI27, IFI44, IFI6, and RSAD2;

(e) IFI27, IFI44, IFI44L, and RSAD2; and

(f) IFI27, IFI44, IFI44L, and IFI6.

### Embodiment 58

The method embodiment 56 or 57 wherein the up-regulated expression or activity of a set of genes is calculated as an average fold increase in the expression or activity of the set of genes.

### Embodiment 59

The method embodiment 58 wherein the average fold increase in the expression or activity of the set of genes is at least about 2, at least about 3, and at least about 4.

### Embodiment 60

The method embodiment 59 wherein the average fold increase in the expression or activity of the set of genes is at least about 2, at least about 2.5, at least about 3, at least about 3.5, at least about 4, at least about 4.5, at least about 5, at least about 5.5, at least about 6, at least about 6.5, at least about 7, at least about 8, at least about 9 or at least about 10.

### Embodiment 61

The method of any one of embodiments 55 to 60 wherein the patient has been diagnosed as having a disorder chosen from lupus, lupus nephritis, dermatomyositis, polymyositis, psoriasis, SSc, vasculitis, sarcoidosis, Sjogren's syndrome, or idiopathic inflammatory myositis.

### Embodiment 62

The method of embodiment 61 wherein the disorder is lupus.

### Embodiment 63

The method of any one of embodiments 55 to 62 wherein the therapeutic agent is a small molecule or a biologic agent.

### Embodiment 64

The method of embodiment 63 wherein the biologic agent is an antibody.

### Embodiment 65

The method of embodiment 64 wherein the antibody is MEDI-545.

### Embodiment 66

The method of embodiment 64 wherein the antibody is specific for one or more type I IFN or IFNα subtype but is not MEDI-545.

### Embodiment 67

The method of any one of embodiments 55 to 66 wherein the up-regulated expression or activity comprises an increase in mRNA levels of one or more of the genes.

### Embodiment 68

The method of any one of embodiments 55 to 66 wherein the up-regulated expression or activity comprises an increase in protein levels of one or more of the genes.

### Embodiment 69

The method of any one of embodiments 55 to 66 wherein the up-regulated expression or activity comprises an increase in enzymatic activity of a protein expressed from one or more of the genes.

### Embodiment 70

The method of any one of embodiments 55 to 69 wherein the sample is whole blood.

### Embodiment 71

A method of diagnosing a patient as a having a disorder associated with increased IFNα levels comprising:

detecting presence or absence of an IFNα-inducible PD marker expression profile in a sample from the patient,

wherein detecting presence of the IFNα-induced PD marker expression profile identifies the patient as having a disorder associated with increased IFNα levels.

### Embodiment 72

The method of embodiment 71 wherein the IFNα-inducible PD marker expression profile comprises up-regulated expression or activity of a set of genes chosen from:

(a) IFI27, IFI44, IFI44L, IFI6, and RSAD2;

(b) IFI44, IFI44L, IFI6, and RSAD2;

(c) IFI27, IFI44L, IFI6, and RSAD2;

(d) IFI27, IFI44, IFI6, and RSAD2;

(e) IFI27, IFI44, IFI44L, and RSAD2; and

(f) IFI27, IFI44, IFI44L, and IFI6.

### Embodiment 73

The method of embodiment 71 wherein the IFNα-inducible PD marker expression profile consists of up-regulated expression or activity of a set of genes chosen from:

(a) IFI27, IFI44, IFI44L, IFI6, and RSAD2;

(b) IFI44, IFI44L, IFI6, and RSAD2;

(c) IFI27, IFI44L, IFI6, and RSAD2;

(d) IFI27, IFI44, IFI6, and RSAD2;

(e) IFI27, IFI44, IFI44L, and RSAD2; and

(f) IFI27, IFI44, IFI44L, and IFI6.

### Embodiment 74

The method embodiment 72 or 73 wherein the up-regulated expression or activity of a set of genes is calculated as an average fold increase in the expression or activity of the set of genes.

### Embodiment 75

The method embodiment 74 wherein the average fold increase in the expression or activity of the set of genes is between at least about at least about 2, at least about 3 and at least about 4.

### Embodiment 76

The method embodiment 74 wherein the average fold increase in the expression or activity of the set of genes is at least about 2, at least about 2.5, at least about 3, at least about 3.5, at least about 4, at least about 4.5, at least about 5, at least about 5.5, at least about 6, at least about 6.5, at least about 7, at least about 8, at least about 9 or at least about 10.

### Embodiment 77

The method of any one of embodiments 72 to 76 wherein the disease or disorder is one of lupus, lupus nephritis, dermatomyositis, polymyositis, psoriasis, SSc, vasculitis, sarcoidosis, Sjogren's syndrome, or idiopathic inflammatory myositis.

### Embodiment 78

The method of embodiment 77 wherein the disorder is lupus.

### Embodiment 79

The method of any one of embodiments 72 to 78 wherein the up-regulated expression or activity comprises an increase in mRNA levels of one or more of the genes.

### Embodiment 80

The method of any one of embodiments 72 to 78 wherein the up-regulated expression or activity comprises an increase in protein levels of one or more of the genes.

### Embodiment 81

The method of any one of embodiments 72 to 78 wherein the up-regulated expression or activity comprises an increase in enzymatic activity of a protein expressed from one or more of the genes.

### Embodiment 82

A method of identifying a candidate therapeutic for treating IFNα-mediated disorders comprising:

contacting cells comprising an IFNα-inducible PD marker expression profile with an agent; and

detecting presence or absence of a change in the IFNα-induced PD marker expression profile of the cells,

wherein the presence of a change comprising a reduction in the up-regulation of the genes of the IFNα-inducible PD marker expression profile indicates the agent is a candidate therapeutic agent.

### Embodiment 83

The method of embodiment 82 wherein the IFNα-inducible PD marker expression profile comprises up-regulated expression or activity of a set of genes chosen from:

(a) IFI27, IFI44, IFI44L, IFI6, and RSAD2;

(b) IFI44, IFI44L, IFI6, and RSAD2;

(c) IFI27, IFI44L, IFI6, and RSAD2;

(d) IFI27, IFI44, IFI6, and RSAD2;

(e) IFI27, IFI44, IFI44L, and RSAD2; and

(f) IFI27, IFI44, IFI44L, and IFI6.

### Embodiment 84

The method of embodiment 82 wherein the IFNα-inducible PD marker expression profile consists of up-regulated expression or activity of a set of genes chosen from:

(a) IFI27, IFI44, IFI44L, IFI6, and RSAD2;

(b) IFI44, IFI44L, IFI6, and RSAD2;

(c) IFI27, IFI44L, IFI6, and RSAD2;

(d) IFI27, IFI44, IFI6, and RSAD2;

(e) IFI27, IFI44, IFI44L, and RSAD2; and

(f) IFI27, IFI44, IFI44L, and IFI6.

### Embodiment 85

The method embodiment 83 or 84 wherein the up-regulated expression or activity of a set of genes is calculated as an average fold increase in the expression or activity of the set of genes.

### Embodiment 86

The method embodiment 85 wherein the average fold increase in the expression or activity of the set of genes is between at least about 3 and at least about 15, between at least about 3 and at least about 10, or between at least about 3 and at least about 5.

### Embodiment 87

The method embodiment 85 wherein the average fold increase in the expression or activity of the set of genes is at least about 2, at least about 2.5, at least about 3, at least about 3.5, at least about 4, at least about 4.5, at least about 5, at least about 5.5, at least about 6, at least about 6.5, at least about 7, at least about 8, at least about 9 or at least about 10.

### Embodiment 88

The method of any one of embodiments 83 to 87 wherein the cells obtained from a patient comprising a disorder associated with increased IFNα levels.

### Embodiment 89

The method of any one of embodiments 83 to 87 wherein the cells are cells treated with IFNα to induce the IFNα-inducible PD marker expression profile.

### Embodiment 90

The method of any one of embodiments 83 to 89 wherein the up-regulation of the genes of the IFNα-inducible PD marker expression profile comprises an increase in mRNA levels of one or more of the genes.

### Embodiment 91

The method of any one of embodiments 83 to 89 wherein the up-regulation of the genes of the IFNα-inducible PD marker expression profile comprises an increase in protein levels of one or more of the genes.

### Embodiment 92

The method of any one of embodiments 83 to 89 wherein the up-regulation of the genes of the IFNα-inducible PD marker expression profile comprises an increase in enzymatic activity of a protein expressed from one or more of the genes.

### Embodiment 93

A set of primers comprising polynucleotides that specifically amplify and detect expression of any one of the following sets of genes:

(a) IFI27, IFI44, IFI44L, IFI6, and RSAD2; or

(b) IFI44, IFI44L, IFI6, and RSAD2; or

(c) IFI27, IFI44L, IFI6, and RSAD2; or

(d) IFI27, IFI44, IFI6, and RSAD2; or

(e) IFI27, IFI44, IFI44L, and RSAD2; or

(f) IFI27, IFI44, IFI44L, and IFI6.

### Embodiment 94

The set of primers of embodiment 93 further comprising primers for amplifying and detecting 18S, ACTB, and GAPDH.

### Embodiment 95

A set of primers consisting of polynucleotides that specifically detect expression of any one of the sets of genes:

(a) IFI27, IFI44, IFI44L, IFI6, and RSAD2; or

(b) IFI44, IFI44L, IFI6, and RSAD2; or

(c) IFI27, IFI44L, IFI6, and RSAD2; or

(d) IFI27, IFI44, IFI6, and RSAD2; or

(e) IFI27, IFI44, IFI44L, and RSAD2; or

(f) IFI27, IFI44, IFI44L, and IFI6.

### Embodiment 96

The embodiments 93 and 94, wherein the set of primers have sequences of SEQ ID NOs 1-24.

### Embodiment 96

Any of embodiments 1-95, wherein IFI27, IFI44, IFI44L, IFI6, and RSAD2 have the sequences of SEQ ID NOs: 25-32.

### Embodiment 97

A kit comprising the primers of embodiments 93-96.

### Embodiment 98

Any of embodiments 1-96, wherein the increased expression is the mean or median increased expression in the mRNA levels of IFI27, IFI44, IFI44L, IFI6, and RSAD2.

### Embodiment 99

A method of identifying a subject suitable for treatment with a therapeutic agent that modulates type 1 interferon activity comprising detecting increased mRNA of at least four of IFI27, IFI44, IFI44L, IFI6, and RSAD2 in a sample of the subject, wherein an increase in mRNA of at least about four fold indicates a subject suitable for treatment with the agent.

### Embodiment 100

The method of embodiment 99, wherein the mRNA is increased relative to the mRNA of at least four of IFI27, IFI44, IFI44L, IFI6, and RSAD2 in pooled samples from healthy patients.

### Embodiment 101

The method of any of embodiments 99 or 100, wherein the increased mRNA is relative to the mRNA of one or more control genes present in the sample.

### Embodiment 102

The method of any of embodiments 99-101, wherein the one or more control genes are chosen from ACTB, GAPDH, and 18S rRNA.

### Embodiment 103

The method of any of embodiment 99-102, wherein increased mRNA of IFI27, IFI44, IFI44L, and RSAD2 is detected.

### Embodiment 104

The method of any of embodiment 99-103, wherein the agent is chosen from an anti-interferon alpha antibody and an anti-interferon alpha receptor antibody.

### Embodiment 105

The method of embodiment of any of 99-104, wherein the anti-interferon antibody is sifalimumab.

### Embodiment 106

The method of embodiment of any of 99-105, wherein the anti-interferon antibody is not sifalimumab.

### Embodiment 107

The method of embodiment of any of 99-106 wherein detecting mRNA of at least IFI27, IFI44, IFI44L, IFI6, and RSAD2 comprises

1) isolating RNA from a sample obtained from the subject;

2) synthesizing cDNA from the RNA;

3) hybridizing the cDNA with oligonucleotides that hybridize to nucleic acid sequences of SEQ ID NOs: 25-32; and

4) amplifying the cDNA and detecting the amplified products.

### Embodiment 108

The method of embodiment of any of 99-107, wherein the oligonucleotides are chosen from oligonucleotides having the sequences of SEQ ID NOs: 13-24.

### Embodiment 109

A method of identifying a subject suitable for treatment with a therapeutic agent that modulates type 1 interferon activity comprising detecting increased mRNA of at least four of IFI27, IFI44, IFI44L, IFI6, and RSAD2 in a sample of the subject, wherein the increased mRNA is calculated according to the following algorithm:

$$Δ {Ct}_{IFN} = \frac{\begin{matrix}( {Ct}_{IFI 44} - {Ct}_{REF} ) + ( {Ct}_{IFI 44 L} - {Ct}_{REF} ) + \\ ( {Ct}_{IFI 27} - {Ct}_{REF} ) + ( {Ct}_{RSAD 2} - {Ct}_{REF} )\end{matrix}}{4} ; wherein$$

$$Δ {Ct}_{REF} = \frac{{Ct}_{ACTB} + {Ct}_{GAPDH} + {Ct}_{18 S}}{3}$$

and wherein a Ct_{IFN} of about 7.6 indicates a subject suitable for treatment with the agent.

### Embodiment 110

The method of embodiment of any of 99-109, wherein the agent is chosen from an anti-interferon alpha antibody and anti-interferon alpha receptor antibody.

### Embodiment 111

The method of embodiment of any of 99-110, wherein the anti-interferon antibody is sifalimumab.

### Embodiment 112

The method of embodiment of any of 99-111, wherein the anti-interferon antibody is not sifalimumab.

### Embodiment 113

The method of embodiment of any of 99-112, wherein detecting the mRNA of at least IFI27, IFI44, IFI44L, IFI6, and RSAD2 comprises:

1) isolating RNA from a sample obtained from the subject;

2) synthesizing cDNA from the RNA

3) hybridizing the cDNA with oligonucleotides that hybridize to nucleic acid sequences of SEQ ID NOs: 25-35, and

4) amplifying the cDNA and detecting the amplified products.

### Embodiment 114

The method of embodiment of any of 99-113, wherein the oligonucleotides are chosen from oligonucleotides having the sequences of SEQ ID NOs: 1-24.

### Embodiment 115

A method for treating a subject with a therapeutic agent that modulates type 1 interferon activity comprising:

a) identifying a subject suitable for treatment by detecting increased mRNA of at least four of IFI27, IFI44, IFI44L, IFI6, and RSAD2 in a sample of the subject, wherein an increase in mRNA of at least about 4 fold indicates a subject suitable for treatment; and

b) administering the therapeutic agent.

### Embodiment 116

The method of embodiment of any of 99-115, wherein the increased mRNA is relative to the mRNA of at least four of IFI27, IFI44, IFI44L, IFI6, and RSAD2 in pooled samples from healthy patients.

### Embodiment 117

The method of embodiment of any of 99-116, wherein the increased mRNA is relative to the mRNA of one or more control genes present in the sample.

### Embodiment 118

The method of embodiment of any of 99-117, wherein the one or more control genes are chosen from ACTB, GAPDH, and 18S rRNA.

### Embodiment 119

The method of embodiment of any of 99-118, wherein increased mRNA of IFI27, IFI44, IFI44L, and RSAD2 is detected.

### Embodiment 120

The method of embodiment of any of 99-119, wherein the agent is chosen from an anti-interferon alpha antibody and anti-interferon alpha receptor antibody.

### Embodiment 121

The method of embodiment of any of 99-120, wherein the anti-interferon antibody is sifalimumab.

### Embodiment 122

The method of embodiment of any of 99-121, wherein the anti-interferon antibody is not sifalimumab.

### Embodiment 123

The method of embodiment of any of 99-122 wherein detecting the mRNA of at least IFI27, IFI44, IFI44L, IFI6, and RSAD2 comprises

1) isolating RNA from a sample obtained from the subject;

2) synthesizing cDNA from the RNA;

3) hybridizing the cDNA with oligonucleotides that hybridize to nucleic acid sequences of SEQ ID NOs: 25-32, and

4) amplifying the cDNA and detecting the amplified products.

### Embodiment 124

The method of embodiment of any of 99-123, wherein the oligonucleotides are chosen from oligonucleotides having the sequences of SEQ ID NOs: 13-24.

### Embodiment 125

A method of identifying a subject suitable for treatment with a therapeutic agent that modulates type 1 interferon activity comprising

a) detecting increased mRNA of at least four of IFI27, IFI44, IFI44L, IFI6, and RSAD2 in a sample of the subject, wherein the increased mRNA is calculated according to the following algorithm:

$$Δ {Ct}_{IFN} = \frac{\begin{matrix}( {Ct}_{IFI 44} - {Ct}_{REF} ) + ( {Ct}_{IFI 44 L} - {Ct}_{REF} ) + \\ ( {Ct}_{IFI 27} - {Ct}_{REF} ) + ( {Ct}_{RSAD 2} - {Ct}_{REF} )\end{matrix}}{4} ; wherein$$

$$Δ {Ct}_{REF} = \frac{{Ct}_{ACTB} + {Ct}_{GAPDH} + {Ct}_{18 S}}{3}$$

and wherein a Ct_{IFN} of about 7.6 indicates a subject suitable for treatment with a therapeutic agent that modulates IFNα activity; and
b) administering the therapeutic agent.

### Embodiment 126

The method of embodiment of any of 99-125, wherein the agent is chosen from an anti-interferon alpha antibody and anti-interferon alpha receptor antibody.

### Embodiment 127

The method of embodiment of any of 99-126, wherein the anti-interferon antibody is sifalimumab.

### Embodiment 128

The method of embodiment of any of 99-127, wherein the anti-interferon antibody is not sifalimumab.

### Embodiment 129

The method of embodiment of any of 99-128, wherein detecting the mRNA of at least IFI27, IFI44, IFI44L, IFI6, and RSAD2 comprises:

1) isolating RNA from a sample obtained from the subject;

2) synthesizing cDNA from the RNA;

3) hybridizing the cDNA with oligonucleotides that hybridize to nucleic acid sequences of SEQ ID NOs: 25-35, and

4) amplifying the cDNA and detecting the amplified products.

### Embodiment 130

The method of embodiment of any of 99-129, wherein the oligonucleotides are chosen from oligonucleotides having the sequences of SEQ ID NOs: 1-24.

All publications, patents and patent applications mentioned in this specification are herein incorporated by reference into the specification to the same extent as if each individual publication, patent or patent application was specifically and individually indicated to be incorporated herein by reference in its entirety.

This application incorporates by reference U.S. Provisional Application Ser. No. 60/924,219 filed May 3, 2007, U.S. Provisional Application Ser. No. 60/924,584 filed May 21, 2007, U.S. Provisional Application Ser. No. 60/960,187 filed Sep. 19, 2007, U.S. Provisional Application Ser. No. 60/996,176 filed Nov. 5, 2007, U.S. Provisional Application Ser. No. 61/129,366 filed Jun. 20, 2008, PCT application PCT/US2007/024947 filed Dec. 6, 2007, PCT application PCT/US2008/62646 filed May 5, 2008, PCT application PCT/US2009/048028 filed Jun. 19, 2009, and U.S. patent application Ser. No. 12/517,333 filed Jun. 2, 2009. This application also incorporates by reference U.S. Provisional Application Ser. No. 60/924,220 filed May 3, 2007, U.S. Provisional Application Ser. No. 60/996,219 filed Nov. 6, 2007, and U.S. Provisional Application Ser. No. 60/996,820 filed Dec. 6, 2007. This application further incorporates by reference U.S. Provisional Application Ser. No. 60/996,174 filed Nov. 5, 2007, PCT application PCT/US2007/024941 filed Dec. 6, 2007 and U.S. patent application Ser. No. 12/517,334 filed Jun. 2, 2009. This application further incorporates by reference U.S. Provisional Application Ser. No. 61/006,963 filed Feb. 8, 2008 and PCT application PCT/US2009/033407 filed Feb. 6, 2009.

The set of examples that follow are provided for the purpose of illustration only and the disclosure should in no way be construed as being limited to these examples.

### EXAMPLES

### IX. Example 1

### Development of a Diagnostic Assay for Anti-IFN Alpha Therapeutics

A. Background

Gene expression profiling was used to identify pharmacodynamic (PD) genes whose transcripts satisfy three selection criteria. Specifically, they are 1) inducible by type 1 IFN subtypes, 2) repressed in SLE patient sera by MEDI-545, and 3) over-expressed in SLE patients compared to normal healthy donors. Such genes include those in International Application No. PCT/US2007/024947, filed Dec. 6, 2007, International Application No. PCT/US2008/062646, filed May 5, 2008, International Application No. PCT/US2009/033407, filed Feb. 6, 2009 and International Application No. PCT/US2009/048028, filed Jun. 19, 2009, each of which is incorporated by reference in its entirety.

Prior studies allowed the identification of groups of genes than can be used as “signatures” of diseases, such as SLE. For example, we previously identified a 21-gene signature for SLE and other autoimmune diseases. See International Application No. PCT/US2007/024947, filed Dec. 6, 2007, which is incorporated by reference in its entirety. We wanted to determine whether a subset of the 21 genes could be reliably used to identify patients suffering from autoimmune diseases, such as SLE. Moreover, we wanted to determine whether a subset of the 21 genes could be used as a signature for whether a patient would respond to a therapeutic agent that modulates type 1 interferon, such as, for example, anti-interferon alpha antibodies or anti interferon receptor antibodies.

To determine whether a subset of the 21 genes could be used to identify patients, we analyzed gene expression data from patients involved in on-going clinical trials of MEDI-545. MEDI-545 was administered to SLE patients following standard clinical protocols. Clinical outcome was assessed using standard SLE evaluation methods such as the Safety of Estrogens in Lupus Erythematosus-Systemic Lupus Erythematosus Disease Activity Index (SELENA-SLEDAI), the British Isles Lupus Activity Group index, and the Physician Global Assessment (MDGA). Gene expression profiling of patient samples was conducted using Affymetrix human genome U133 plus 2.0 GeneChips® to identify candidate genes.

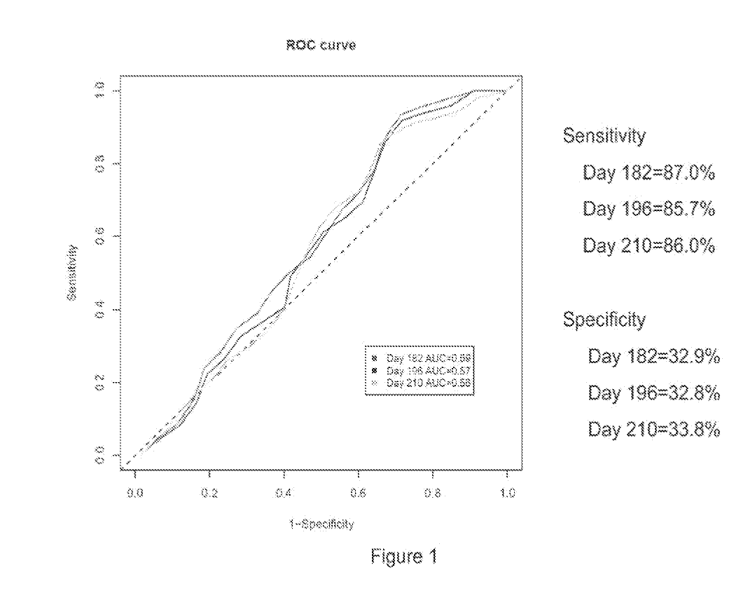

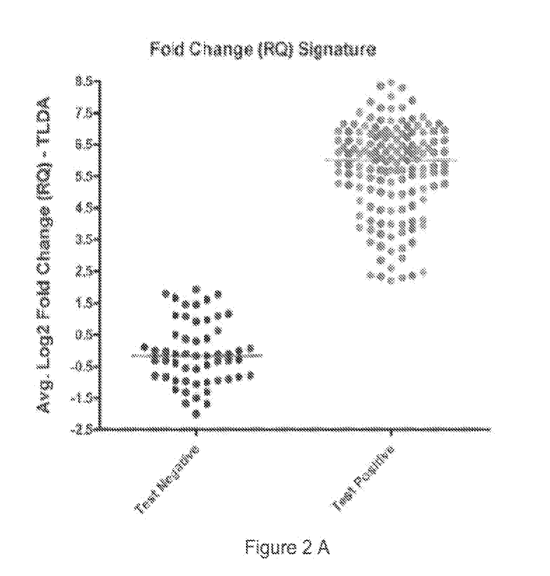

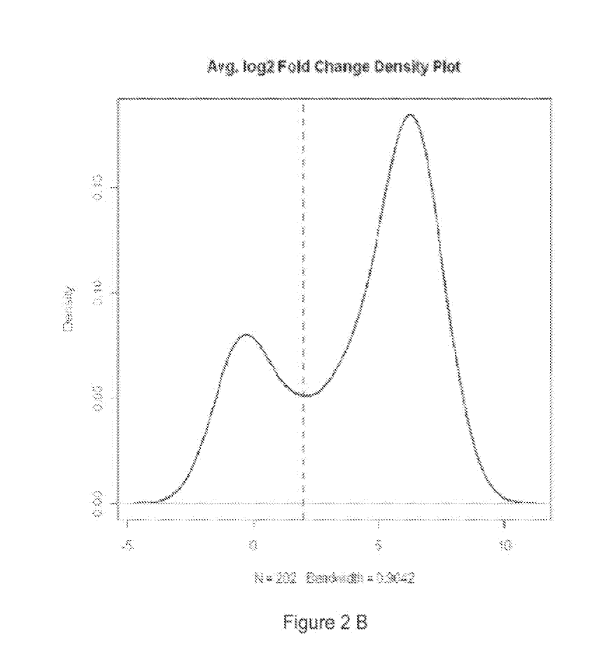

As discussed below, this analysis led to the identification of a set of diagnostic genes (e.g., 4 or 5 gene based assay) for the identification of patients (e.g., SLE or myositis patients) for treatment with an anti-IFN alpha or type I interferon therapeutic. Specifically, the diagnostic gene set was selected for its ability to predict response to sifalimumab (MEDI-545) in SLE patients. As a result, measurement of the expression of these four genes can be used to predict SLE patients who will or will not benefit from treatment with sifalimumab. As detailed below, expression of the four genes may be used in a variety of conditions and to identify patients suitable for treatment with therapies directed against those diseases. A group of five genes suitable for a diagnostic assay consists of IFI44, IFI44L, IFI27, RSAD2 and IFI6. Any 4 genes selected from this group may be suitable for a diagnostic assay. The group of genes for which analytical data is provided in Table 1 and FIGS. 1 and 2 consists of IFI27, IFI44, IFI44L, and RSAD2.

B. Assay Principle

The diagnostic assay is based on quantitative Reverse Transcription-Polymerase Chain Reaction (qRT-PCR). The assay amplifies and detects transcripts originating from four target genes: IFI44, IFI44L, IFI27 and RSAD2. The diagnostic assay also amplifies and detects a set of endogenous RNA molecules as controls (“housekeeping genes”): ACTB, GAPDH, and 18S rRNA. The transcript targets are amplified from total RNA samples obtained from whole blood samples collected from patients using the cleared PAXgene™ Blood RNA System (Qiagen, kit Cat #762164; Becton Dickinson, collection tubes Cat #762165; K042613). RNA is isolated using the procedures specified in the PAXgene™ Blood RNA kit. The target transcripts are amplified using two sequence-specific forward and reverse primers (unlabeled). Each amplified target is detected using a sequence-specific probe that is labeled with a fluorescent reporter moiety, FAM, and a fluorescence quenching moiety, BHQ1. Each target is amplified and detected in individual wells of a 96 well plate. A quantitative determination of the amount of each transcript in a sample is made based on detection of fluorescence on the Applied Biosystems 7500 Fast Dx Real-Time PCR Instrument, a Clinical Multiplex Test System (Applied Biosystems, Foster City, Calif., Cat #4406985; K082562) with SDS software version 1.4.

The quantitative measurement results in the determination of a Cycle Threshold (Ct) value for each target that corresponds to the relative abundance of the transcript in the RNA sample. The Ct is determined by measuring the geometric increase in the fluorescence signal that results from the release of reporter-quencher proximity by the 5′-3′ exonuclease activity of the polymerase during the elongation phase of each amplification cycle. The quantitative Ct values for each gene and control are used to calculate the level of overexpression of target genes in subject suffering from autoimmune diseases relative to healthy subjects. The level of overexpression may be expressed as a mean fold change in mRNA level of the target genes in a subject suffering from an autoimmune disease relative to the mean level of the genes in a healthy subject of or it may be expressed as a score of overexpression of the Type I Interferon-inducible genes.

Specifically, the score (Ct_{IFN}) (“delta Ct”) is calculated as the difference between the mean Ct for the four target genes and the mean Ct of the three control genes according to the following algorithm:

$$Δ {Ct}_{IFN} = \frac{\begin{matrix}( {Ct}_{IFI 44} - {Ct}_{REF} ) + ( {Ct}_{IFI 44 L} - {Ct}_{REF} ) + \\ ( {Ct}_{IFI 27} - {Ct}_{REF} ) + ( {Ct}_{RSAD 2} - {Ct}_{REF} )\end{matrix}}{4} ; where$$

$$Δ {Ct}_{REF} = \frac{{Ct}_{ACTB} + {Ct}_{GAPDH} + {Ct}_{18 S}}{3}$$

This score is then used to determine a qualitative test result in which a score greater than or equal to a cutoff is a positive test result and a score less than a cutoff is a negative test result. A negative result indicates that a patient is not likely to respond to sifalimumab treatment (Non-Responder) and a positive result indicates that a patient is likely to respond to sifalimumab treatment (Responder).

C. Assay Steps

### 1. RNA Preparation

Total RNA is prepared from whole blood, using the cleared PAXgene™ Blood RNA System (Qiagen, kit Cat #762164; Becton Dickinson, collection tubes Cat #762165; K042613) and provides the template for cDNA synthesis. RNA yield and quality is tested by spectrophotometry by measuring the absorbance at wavelengths of 260 nm and 280 nm RNA yield is calculated using the formula C (mg/ml)=A280/0.025. In addition, the purity of RNA is evaluated using the ratio of the absorbance at 260 nm and 280 nm. The acceptable range of A260/A280 ratio is ≧1.6.

2. cDNA Synthesis and Target Amplification

cDNA is synthesized from purified total RNA using the liquid RT Enzyme Mix and the RT Buffer containing the RT primers and nucleotides. Synthesis of cDNA uses a random priming approach using RT primers that are a mixture of random hexadecamers that hybridize to the RNA molecules and serve as a substrate for the RT Enzymes. The resulting cDNA contains a proportional mixture of DNA representing the sequences present in the RNA sample.

The target transcripts are simultaneously amplified and detected using the qPCR buffer, the gene primer and probes and clinical grade AmpliTaq Gold polymerase. During each amplification cycle, two sequence-specific forward and reverse primers (unlabeled) hybridize to complementary cDNA templates during the annealing phase and serve as a substrate for the AmpliTaq Gold polymerase during the elongation phase, resulting in the production of a new DNA strand complementary to the target. The forward and reverse primers each hybridize to a different sense or antisense strand. Detection of each amplified target is accomplished using a sequence-specific probe that is labeled with a fluorescent reporter moiety, FAM, and a fluorescence quenching moiety, BHQ1, that hybridizes to the complementary target sequence during the annealing phase. Each target is amplified and detected in individual wells of a 96 well plate.

Thermal Cycling Protocol

| 37 C. for 15 min | |
|---|---|
| 95 C. for 10 min | |
| 95 C. for 15 sec | 50 cycles |

60 C. for 1 min
7500 thermal cycler run mode: Standard

### 3. Signal Detection

A quantitative determination of the amount of each transcript in a sample is made based on detection of fluorescence on the Applied Biosystems 7500 Fast Dx Real-Time PCR Instrument (Applied Biosystems, Foster City, Calif., Cat #4406985; K082562) with SDS software version 1.4, which results in the determination of a Cycle Threshold (Ct) value for each target that corresponds to the relative abundance of the transcript in the RNA sample. The instrument determines the Ct by measuring the geometric increase in the fluorescence signal that results from the release of reporter-quencher proximity by the 5′-3′ exonuclease activity of the polymerase during the elongation phase of each amplification cycle. The quantitative Ct values for each gene and control are used to calculate a score of overexpression of the Type I Interferon-inducible genes.

### 4. Assay Controls

In vitro Transcript (IVT) RNA controls (each of the four target genes and the housekeeping gene controls) optionally included with each run are designed to detect potential user error, contamination, cross-reactions and/or assay failure during the Reverse Transcription, cDNA synthesis, PCR, hybridization, and/or detection steps. The control results are used as validity controls to validate or invalidate a given run. They are not used to validate the RNA isolation, which is performed with the cleared PAXgene™ Blood RNA System and controlled by a RNA quality check. A Negative and a low Positive control, close to the cutoff, may be included provided.

### 5. Software

Software is used to calculate the delta Ct using the equation described above. The delta Ct is then used to determine a qualitative test result in which a score greater than or equal to a cutoff is a positive test result and a score less than the cutoff is a negative test result. A negative result indicates that a patient is not likely to respond to sifalimumab treatment (Non-Responder) and a positive result indicates that a patient is likely to respond to sifalimumab treatment (Responder).

### 6. Assay Kit Components

| Usage | Components |
|---|---|
| RT Reagents | RT Buffer |
| | RT Enzyme mix |
| | Diluent |
| DNA | qPCR buffer |
| Amplification | Oligonucleotide primers and probes for IFI44, IFI44L, |
| Reagents | IFI27 RSAD2 and |
| | Oligonucleotide primers and probes for Three Control |
| | Genes (18S, ACTB, and GAPDH) |
| | AmpliTaq Gold Polymerase |
| Controls | Positive Control |
| | Negative Control |
| | NTC |

### 7. Oligonucleotides for Simultaneously Amplifying and Detecting Target Genes

| Oligo Name | Target | SEQ ID NO: | Sequence |
|---|---|---|---|
| MEDI_0001-F | 18S | SEQ ID NO: 1 | GCTACCACAT |
| | | | CCAAGGAAGG |
| MEDI_0001-P | | SEQ ID NO: 2 | CGCAAATTAC |
| | | | CCACTCCCGA |
| | | | CCC |
| MEDI_0001-R | | SEQ ID NO: 3 | GCCTCGAAAG |
| | | | AGTCCTGTAT |
| | | | TG |
| MEDI_0002-F | ACTB | SEQ ID NO: 4 | ACAGAGCCTC |
| | | | GCCTTTG |
| MEDI_0002-P | | SEQ ID NO: 5 | AGCTGGCGGC |
| | | | GGGTGTGG |
| MEDI_0002-R | | SEQ ID NO: 6 | CCTTGCACAT |
| | | | GCCGGAG |
| MEDI_0003_A-F | GAPDH | SEQ ID NO: 7 | ACATCGCTCA |
| | | | GACACCATG |
| MEDI_0003_A-P | | SEQ ID NO: 8 | CCGTTGACTC |
| | | | CGACCTTCAC |
| | | | CTT |
| MEDI_0003_A-R | | SEQ ID NO: 9 | ACCAGAGTTA |
| | | | AAAGCAGCCC |
| MEDI_0003_B-F | GAPDH | SEQ ID NO: 10 | TGGGTGTGAA |
| | | | CCATGAGAAG |
| | | | TATG |
| MEDI_0003_B-P | | SEQ ID NO: 11 | CCTCAAGATC |
| | | | ATCAGCAATG |
| | | | CCTCCTGCA |
| MEDI_0003_B-R | | SEQ ID NO: 12 | CAGGGGTGCT |
| | | | AAGCAGTTGG |
| MEDI_0004-F | IFI27 | SEQ ID NO: 13 | CTCTCACCTC |
| | | | ATCAGCAGTG |
| MEDI_0004-P | | SEQ ID NO: 14 | CCAGAGGCCA |
| | | | CCCTGACCAC |
| MEDI_0004-R | | SEQ ID NO: 15 | TCACAACTGT |
| | | | AGCAATCCTG |
| | | | G |
| MEDI_0005-F | IFI44 | SEQ ID NO: 16 | GATGCGAAGA |
| | | | TTCACTGGAT |
| | | | G |
| MEDI_0005-P | | SEQ ID NO: 17 | AGTTCTCAAG |
| | | | GCAGACAGTA |
| | | | AGCTCTTC |
| MEDI_0005-R | | SEQ ID NO: 18 | TGTTGAACCA |
| | | | GGGATCCATA |
| | | | TG |
| MEDI_0006-F | IFI44L | SEQ ID NO: 19 | AAGCCGTAGT |
| | | | GGGGTCT |
| MEDI_0006-P | | SEQ ID NO: 20 | TATACCGCTC |
| | | | GGTTATGCTG |
| | | | GTG |
| MEDI_0006-R | | SEQ ID NO: 21 | AACATAAATG |
| | | | GCAGAGATTT |
| | | | TCCA |
| MEDI_0007-F | RSAD2 | SEQ ID NO: 22 | AAAGACTCCT |
| | | | ACCTTATTCT |
| | | | GGATG |
| MEDI_0007-P | | SEQ ID NO: 23 | CTGAACTGTA |
| | | | GAAAGGGACG |
| | | | GAAGGAC |
| MEDI_0007-R | | SEQ ID NO: 24 | CTTCTACACC |
| | | | AACATCCAGG |
| | | | A |

### 8. Sequence of Target Genes

| Target Name | SEQ ID NO |
|---|---|
| IFI27 variant 1 | SEQ ID NO: 25 |
| IFI27 variant 2 | SEQ ID NO: 26 |
| IFI44 | SEQ ID NO: 27 |
| IFI44L | SEQ ID NO: 28 |
| IFI6 variant 1 | SEQ ID NO: 29 |
| IFI6 variant 2 | SEQ ID NO: 30 |
| IFI6 variant 3 | SEQ ID NO: 31 |
| RSAD2 | SEQ ID NO: 32 |
| 18S | SEQ ID NO: 33 |
| ACTB | SEQ ID NO: 34 |
| GAPDH | SEQ ID NO: 35 |

### X. Example 2

### A Variety of Analyses Demonstrate that Response to MEDI-545 Correlates with the Four Gene Signature

The threshold value used to designate a diagnostic positive versus negative subject was determined using two primary methods. First, the distribution of the four gene diagnostic fold change score from 202 SLE subjects was evaluated for the presence of modes beyond a single mode. Such a multi-mode distribution would imply the presence of more than one score population. From this distribution, two distinct modes were identified and the centroid of the region that discriminated these two modes was found at a value of approximately 4. The distribution shows that a range about to 2 to about 8 may be used to discriminate the two modes and thus a suitable mean fold change cutoff may be between about 2 and about 8.

Second, the four gene diagnostic fold change score distribution of 24 normal healthy donors was evaluated for upper limits in comparison to the SLE score distribution. The average diagnostic fold change score from the 24 normal healthy donors is 1.34 with the upper bound (mean+2*SD) of 2.91. Since the subject count in the normal healthy donor population was much smaller than the subject counts in our disease population, we used a conservative estimate of the upper bound diagnostic score in the normal healthy donors that agreed with the results from the bimodal SLE score distribution. As such, a cut point of 4 was selected for stratifying SLE patients in diagnostic positive and diagnostic negative groups.

For the diagnostic positive population, 147 SLE subjects were stratified into this group and the mean, median, and standard deviation for the scores are 36.13, 63.76, and 2.71, respectively. For the diagnostic negative population, 55 SLE subjects were stratified in this group and mean, median, and standard deviation for the scores are 0.95, 0.89, and 2.00, respectively. A two-sample two-tailed Welch's modified t-test indicates a significant difference between the two groups at a p-value of 1.62×10⁻⁶⁶.

Receiver Operator Characteristic (ROC) curves using SLEDAI clinical endpoint were generated using the four gene signature (i.e. IFI27, IFI44, IFI44L, and RSAD2 signature) obtained from the clinical trial data based on the fold-change calculation. The curve is shown in FIG. 1 using a SLEDAI drop of at least 4 points from baseline as the endpoint, evaluated at days 182, 196, and 210 post-treatment. Based on the ROC curve, a cutoff point of mean fold change of 4 in expression for IFI27, IFI44, IFI44L, and RSAD2 was selected.

With a cutoff point of mean fold change 4 the sensitivity and specificity of the test is 87.0% and 32.9%, respectively, based on data obtained from blood samples collected on day 182 post treatment. The AUC for day 182 data is 0.59. With a cut point of mean fold change ≧4 the sensitivity and specificity of the test is 85.7% and 32.8%, respectively, based on data obtained from blood samples collected on day 196 post treatment. The AUC for day 196 data is 0.57. Finally, when using the same cutoff point, the sensitivity, specificity, and AUC values obtained at day 210 post treatment are 86.0%, 33.8%, and 0.56, respectively.

When using the mean of overexpression shown as a fold-change for each of the four genes as a classifier to partition SLE patients into Diagnostic Positive (Responder) versus Diagnostic Negative (Non-Responder) status, there is a clear separation between groups. The distribution of the four gene mean scores on a log 2 scale clearly exhibits two apparent modes, with few values falling between them. FIGS. 2A and B show the distribution of patients into diagnostic test positive and negative groups using the four gene (IFI27, IFI44, IFI44L, and RSAD2) diagnostic with a cut off of mean fold change of

A. SLENA-SLEDAI ≧4

We pooled all sifalimumab dose groups across MI-CP 152, and used the clinical endpoint of reduction in SELENA-SLEDAI ≧4 points from baseline (SELENA-SLEDAI Responder), to calculate at Day 182 a predictive value positive of 46.6% (PPV; percentage of subject Responders among Diagnostic-Positive subjects [Dx+]). Similarly, we calculated a predictive value negative at Day 182 of 82.1% (NPV; percentage of subject Non-Responders among Diagnostic-Negative subjects [Dx−]). For this same clinical endpoint, the sensitivity and specificity values were 87.0% and 32.9%.

Because we observed a placebo response rate of approximately 20% to 40%, we found it appropriate to calculate the “delta PPV” and “delta NPV,” to factor in the placebo effect on the accuracy rates. The delta PPV value is the difference between the PPV for sifalimumab-treated subjects (based on mostly 0.3 and 1.0 mg/kg data available at the time of the interim analysis) and the PPV for placebo-treated subjects. Similarly, for the delta NPV, this calculation consisted of the difference between the NPV for sifalimumab-treated subjects (again mostly 0.3 and 1.0 mg/kg group data) and the NPV for placebo-treated subjects. These delta PPV/NPV statistics are conservative estimates of the PPV and NPV and demonstrate the percentage of responding patients given drug, in the context of those patients that respond given placebo. As such, these statistics show the inherent ‘noise’ level in responder status. Even with these adjusted PPV/NPV values, however, we still see a strong response rate for Dx positive patients treated with Sifalimumab as compared to Dx negative patients treated with Sifalimumab.

A summary of results is shown in Table 1.

TABLE 1
Response rates stratified by diagnostic status, reported at days 182, 196, and 210 post treatment

| | SELENA-SLEDAI | | | | | | |
|---|---|---|---|---|---|---|---|
| Treatment | | Responders | Day 182 | Responders | Day 196 | Responders | Day 210 |
| Group | DX Status | at day 182 | 95% CI | at day 196 | 95% CI | at day 210 | 95% CI |
| Sifalimumab | Positive | 46.6% (88) | 36.5% 56.9% | 48.9% (88) | 38.7% 59.1% | 50.6% (87) | 40.3% 60.8% |
| | Negative | 17.9% (28) | 7.9% 35.6% | 21.4% (28) | 10.2% 39.5% | 21.4% (28) | 10.2% 39.5% |
| Placebo | Positive | 32.1% (28) | 17.9% 50.7% | 39.3% (28) | 23.6% 57.6% | 37.0% (27) | 21.5% 55.8% |
| | Negative | 11.1% (9) | 2.0% 13.5% | 22.2% (9) | 6.3% 4.7% | 11.1% (9) | 2.0% 3.5% |
| Sifalimumab − | Positive | 14.4% | −5.7% 34.6% | 9.6% | −11.3% 30.5% | 13.5% | −7.5% 34.6% |
| Placebo | Negative | 6.7% | −36% 34.8% | −0.8% | −31.9% 30.3% | 10.3% | −15.2% 35.9% |

CI = confidence interval;
DX = diagnostic

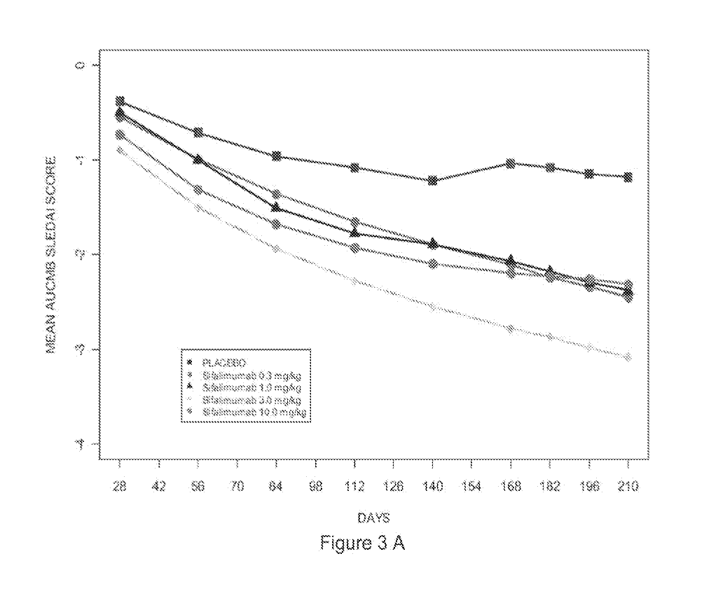

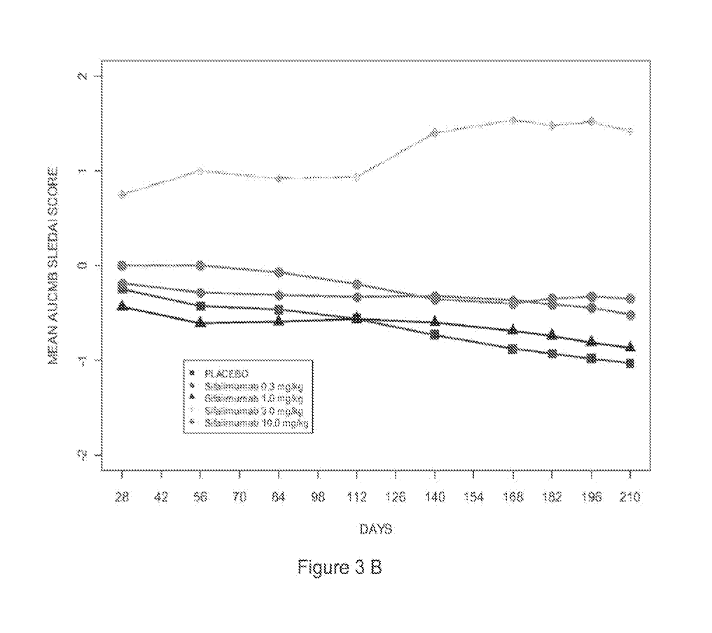

FIGS. 3 A and B show data from a phase 1b, multicenter, randomized, double-blinded, placebo-controlled, dose-escalation study to evaluate multiple intravenous doses of MEDI-545 in patients with moderately to severely active SLE. All SLE subjects have SLEDAI score ≧6 at prescreening. FIGS. 3A and B shows the time adjusted area under the curve minus baseline SLEDAI score in four gene (IFI27, IFI44, IFI44L, and RSAD2) signature positive or negative SLE patients in placebo, or 0.3/1/3/10 mg/kg of MEDI-545 cohorts. FIG. 3A signature positive, FIG. 3B signature negative.

There are no major correlation patterns between the four gene diagnostic (i.e. IFI27, IFI44, IFI44L, and RSAD2 signature) and the primary demographic/clinical variables of the subjects in the clinical trials. Pearson's correlation coefficients are provided in Table 2 for these primary variables.

TABLE 2
Pearson's correlation coefficients for the four gene signature
(i.e. IFI27, IFI44, IFI44L, and RSAD2 signature) and the primary
demographic/clinical variables. Day 182 and day 196 represents
gene expression patterns obtained from blood collected on day
182 and 196, respectively, following MEDI545 administration.

| Demographic/Clinical variables | Day 182 | Day 196 |
|---|---|---|
| Age | −0.04 | −0.04 |
| Weight | 0.11 | 0.07 |
| Sex | 0.11 | 0.10 |
| Race | −0.09 | −0.09 |
| Ethnicity | −0.14 | −0.14 |
| Country | −0.11 | −0.11 |
| Baseline steroid use | 0.17 | 0.13 |

B. Positive Correlation in SELDAI Responses, Reduction (Improvement) ≧4 Points.

Data are from trial CP152. Subjects with moderately to severely active SLE were stratified by screening type I IFN signature and then exposed to placebo or sifalimumab from days 0-182. A SLEDAI response is shown, which is SLEDAI reduction (improvement in disease activity) ≧4 points. Solid squares/line represent sifalimumab exposed subjects and open squares/dotted lines represent placebo patients. Short lines on days 182-210 only are average values for days 182-210, with no symbols, solid line representing average value for sifalimumab subjects and no symbols, dotted lines representing average values for placebo subjects. Subjects who received at least one dose of placebo or sifalimumab and who had a baseline SLEDAI score ≧6 are included, with data shown for subjects receiving 1, 3, or 10 mg/kg sifalimumab or placebo. Subjects who required rescue corticosteroids at levels greater than that allowed in the protocol for an increase in disease activity on or before day 196 are considered non-responders in this analysis.

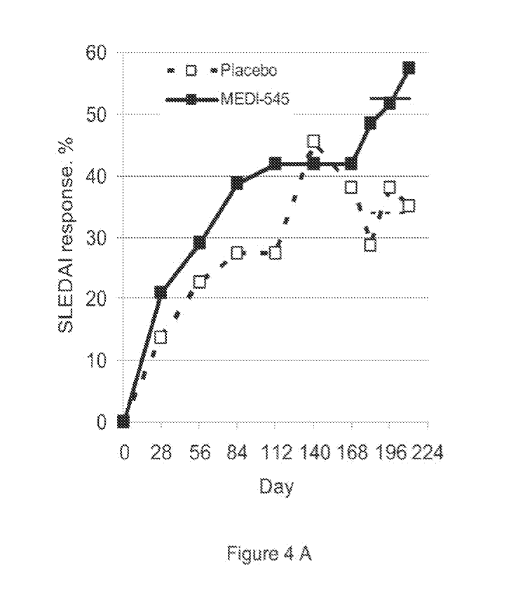

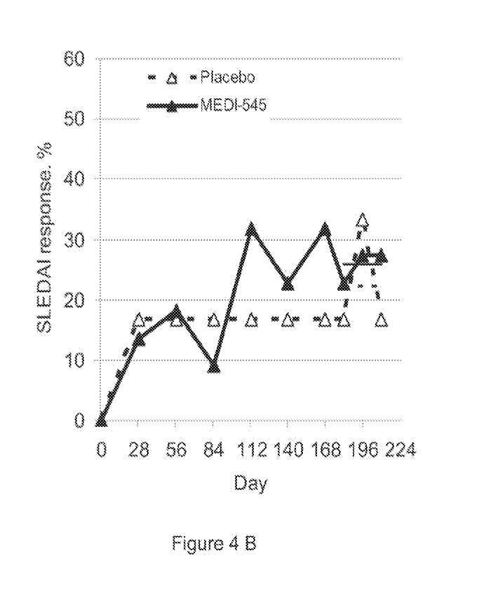

FIG. 4 A includes subjects with a positive diagnostic test at screening, with average SLEDAI response (%) on days 182-210 52.5% for sifalimumab subjects (n=88) and 33.9% for placebo subjects (n=22). FIG. 4 B includes subjects with a negative diagnostic test at screening, with average composite response on days 182-210 25.7% for sifalimumab subjects (n=22) and 22.2% for placebo subjects (n=6).

C. Positive Correlation in Reduction of Signature and SLEDAI Response. SLEDAI Responses, Reduction (Improvement) ≧4 Points, in Dx+ Subjects with ≧50% Reduction Vs. <50% Reduction in Dx Post Baseline

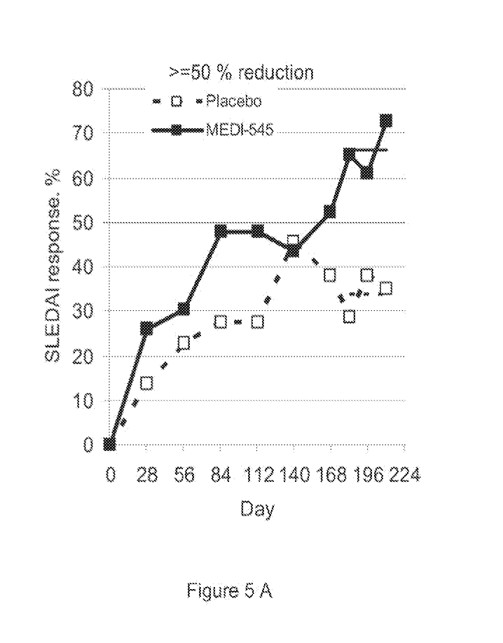

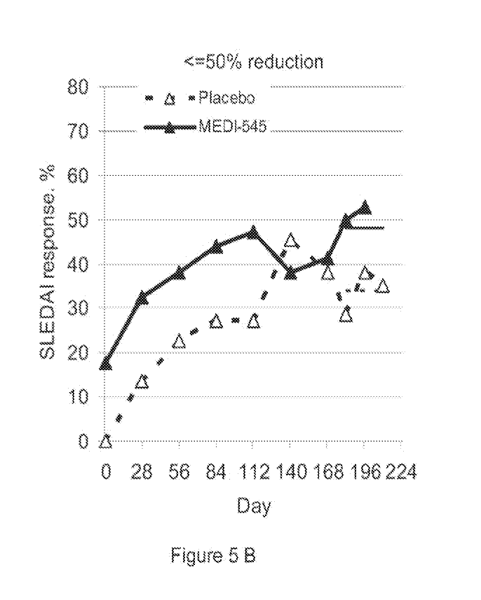

FIGS. 5 A and B. Data are from trial CP152. Subjects with moderately to severely active SLE were stratified by screening type I IFN signature and then exposed to placebo or sifalimumab from days 0-182. A SLEDAI response is shown, which is SLEDAI reduction (improvement in disease activity) >4 points. Solid squares/line represent sifalimumab exposed subjects and open squares/dotted lines represent placebo patients. Short lines on days 182-210 only are average values for days 182-210, with no symbols, solid line representing average value for sifalimumab subjects and no symbols, dotted lines representing average values for placebo subjects. Subjects who received at least one dose of placebo or sifalimumab and a baseline SLEDAI score >6 are included, with data shown for subjects receiving 1, 3, or 10 mg/kg sifalimumab or placebo. Subjects who required rescue corticosteroids at levels greater than that allowed in the protocol for an increase in disease activity on or before day 196 are considered non-responders in this analysis.

FIG. 5 A includes subjects with a positive diagnostic test at screening and, if sifalimumab treated, had a >50% median reduction in baseline diagnostic signature score post dosing days 28-210, with average SLEDAI response (%) on days 182-210 66.3% for sifalimumab subjects (n=23) and 33.9% for placebo subjects (n=22). Figure B includes subjects with a positive diagnostic test at screening, and, if sifalimumab treated, had a <50% median reduction in baseline diagnostic signature score post dosing days 28-210, with average SLEDAI response on days 182-210 48.0% for sifalimumab subjects (n=23) and 33.9% for placebo subjects (n=22).

D. Positive Correlation in Composite Response and Signature

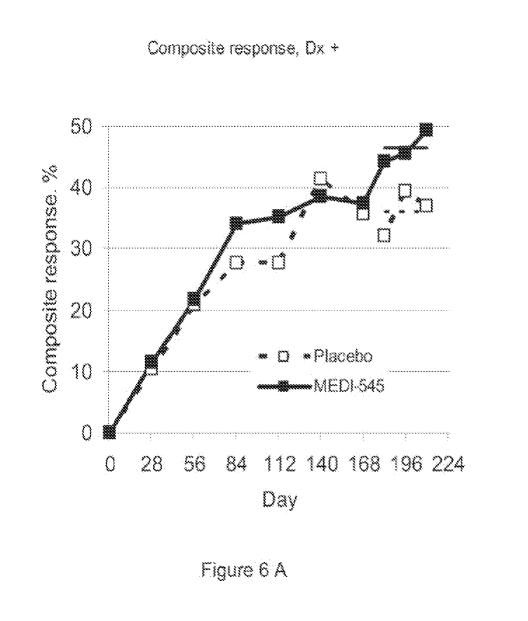

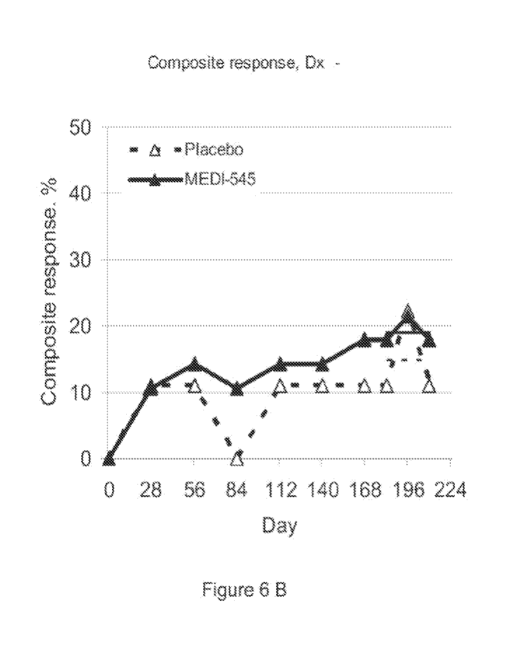

As shown in FIGS. 6 A and B, the composite response of subjects correlates with a positive four gene signature score. Data are from trial CP152. Subjects with moderately to severely active SLE were stratified by screening type I IFN signature and then exposed to placebo or sifalimumab at doses of 0.3, 1, 3, or 10 mg/kg IV every 2 weeks, from days 0-182. A composite response is shown, which is SLEDAI reduction (improvement) ≧4 points+no more than 1 new BILAG B score (a measure of at least moderate flare)+no worsening in physician global assessment >0.3 inches on 3 inch visual analogue scale. Solid squares/line represent sifalimumab exposed subjects and open squares/dotted lines represent placebo patients. Short lines on days 182-210 only are average values for days 182-210, with no symbols, solid line representing average value for sifalimumab subjects and no symbols, dotted lines representing average values for placebo subjects. Subjects who received at least one dose of placebo or sifalimumab and who had a baseline SLEDAI score >6 are included. Subjects who required rescue corticosteroids at levels greater than that allowed in the protocol for an increase in disease activity on or before day 196 are considered non-responders in this analysis. FIG. 6 A includes subjects with a positive diagnostic test at screening, with average composite response (%) on days 182-210 46.4% for sifalimumab subjects (n=88) and 36.1% for placebo subjects (n=29). FIG. 6 B includes subjects with a negative diagnostic test at screening, with average composite response on days 182-210 19.1% for sifalimumab subjects (n=28) and 14.8% for placebo subjects (n=9).

E. Positive Correlation in SLEDAI Area Under the Curve Minus Baseline

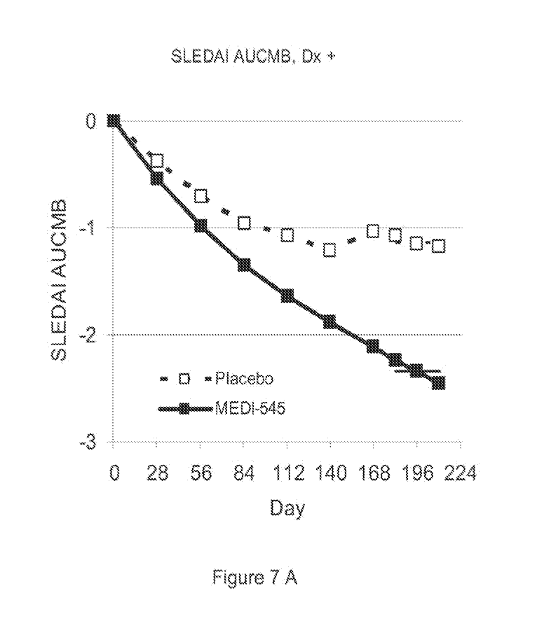

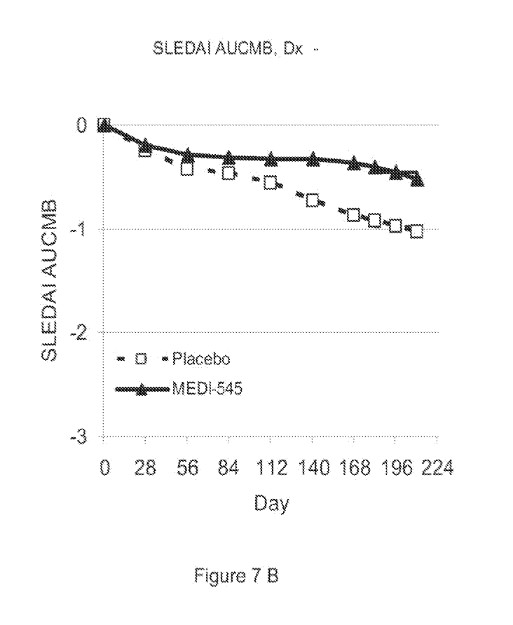

As shown in FIGS. 7 A and B, reduction in SLEDAI area under the curve minus baseline correlates with a positive four gene signature score. Data are from trial CP152. Subjects with moderately to severely active SLE were stratified by baseline type I IFN signature and then exposed to placebo or sifalimumab at doses of 0.3, 1, 3, or 10 mg/kg IV every 2 weeks, from days 0-182. The area under the curve minus baseline for SLEDAI score (a measure of disease activity) is shown, with greater negative values indicating greater reduction in disease activity. Solid squares/line represent sifalimumab exposed subjects and open squares/dotted lines represent placebo patients. Short lines on days 182-210 only are average values for days 182-210, with no symbols, solid line representing average value for sifalimumab subjects and no symbols, dotted lines representing average values for placebo subjects. Subjects who received at least one dose of placebo or sifalimumab are included. FIG. 7 A includes subjects with a positive diagnostic test at screening, with average SLEDAI area under the curve minus baseline on days 182-210 of −2.34 for sifalimumab subjects (n=92) and −1.14 for placebo subjects (n=30). FIG. 7 B includes subjects with a negative diagnostic test at screening, with average SLEDAI area under the curve minus baseline on days 182-210 of −0.46 for sifalimumab subjects (n=29) and −0.88 for placebo subjects (n=10).

F. Positive Correlation in SLEDAI Change from Baseline and Signature

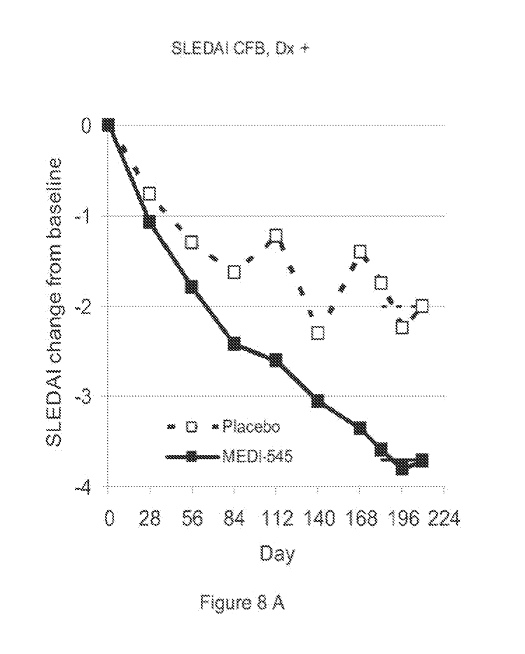

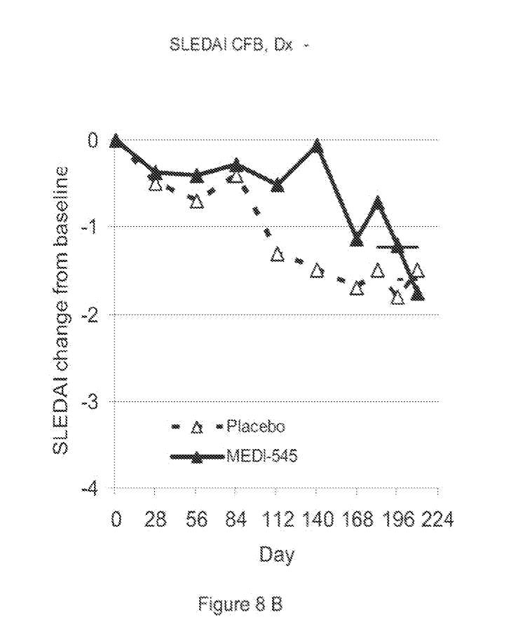

As shown in FIGS. 8 A and B reduction in SLEDAI from baseline correlated with a positive four gene signature score. Data are from trial CP152. Subjects with moderately to severely active SLE were stratified by screening type I IFN signature and then exposed to placebo or sifalimumab at doses of 0.3, 1, 3, or 10 mg/kg IV every 2 weeks, from days 0-182. A SLEDAI response is shown, which is SLEDAI reduction (improvement in disease activity) ≧4 points, in subjects with baseline SLEDAI ≧6. Solid squares/line represent sifalimumab exposed subjects and open squares/dotted lines represent placebo patients. Short lines on days 168-210 only are average values for days 168-210, with no symbols, solid line representing average value for sifalimumab subjects and no symbols, dotted lines representing average values for placebo subjects. Subjects who received at least one dose of placebo or sifalimumab. Subjects who required rescue corticosteroids at levels greater than that allowed in the protocol for an increase in disease activity on or before day 196 are considered non-responders in this analysis. Subjects with a positive diagnostic test at baseline and with median reduction in type I IFN signature of ≧50% post dosing from days 28-210 are shown. FIG. 8 A includes subjects who received sifalimumab at 1 mg/kg IV with average SLEDAI response days 168-210 of 53% for sifalimumab subjects (n=10) and 21% for placebo subjects from the same cohort (n=7). FIG. 8 B includes subjects who received sifalimumab at 3 mg/kg IV with average SLEDAI response days 168-210 of 63% for sifalimumab subjects (n=6) and 33% for placebo subjects from the same cohort (n=6).

G. Inhibition of Signature, SLEDAI Response, and Dose

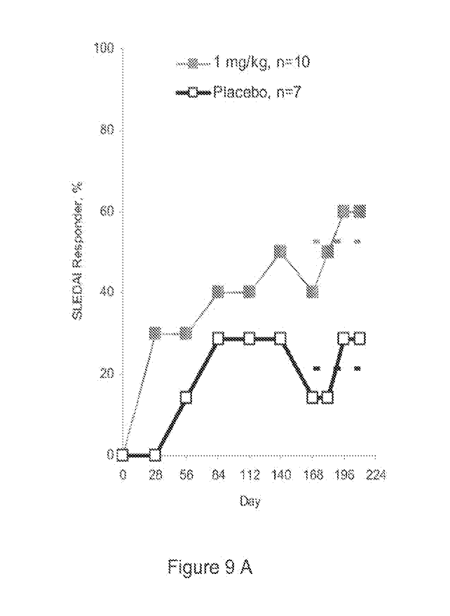

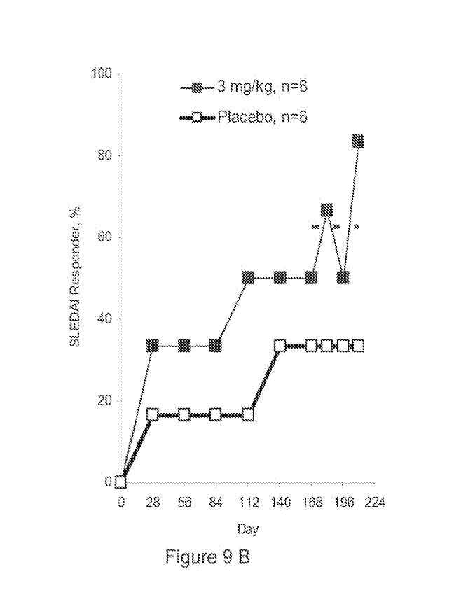

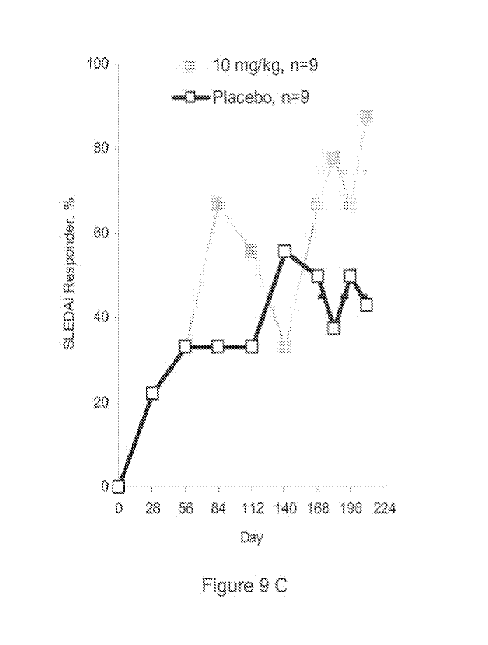

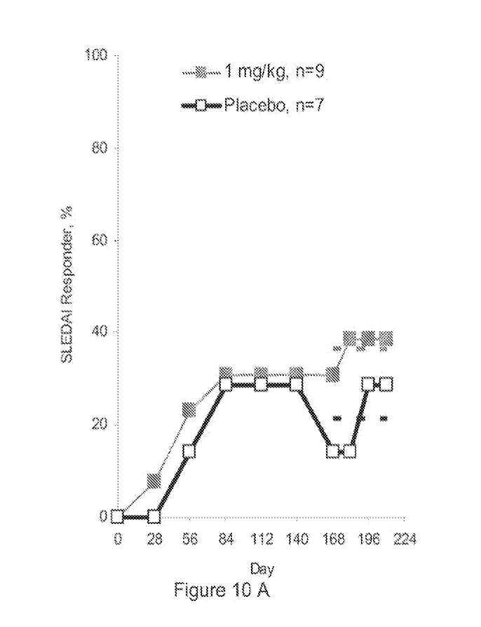

FIGS. 9 A-C. Data are from trial CP152. Subjects with moderately to severely active SLE were stratified by screening type I IFN signature and then exposed to placebo or sifalimumab at doses of 0.3, 1, 3, or 10 mg/kg IV every 2 weeks, from days 0-182. A SLEDAI response is shown, which is SLEDAI reduction (improvement in disease activity) ≧4 points, in subjects with baseline SLEDAI ≧6. Solid squares/line represent sifalimumab exposed subjects and open squares/dotted lines represent placebo patients. Short lines on days 168-210 only are average values for days 168-210, with no symbols, solid line representing average value for sifalimumab subjects and no symbols, dotted lines representing average values for placebo subjects. Subjects who received at least one dose of placebo or sifalimumab. Subjects who required rescue corticosteroids at levels greater than that allowed in the protocol for an increase in disease activity on or before day 196 are considered non-responders in this analysis. Subjects with a positive diagnostic test at baseline and with median reduction in type I IFN signature of ≧50% post dosing from days 28-210 are shown. FIG. 9 A includes subjects who received sifalimumab at 1 mg/kg IV with average SLEDAI response days 168-210 of 53% for sifalimumab subjects (n=10) and 21% for placebo subjects from the same cohort (n=7). FIG. 9 B includes subjects who received sifalimumab at 3 mg/kg IV with average SLEDAI response days 168-210 of 63% for sifalimumab subjects (n=6) and 33% for placebo subjects from the same cohort (n=6). FIG. 9 C includes subjects who received sifalimumab at 10 mg/kg IV with average SLEDAI response days 168-210 of 75% for sifalimumab subjects (n=9) and 45% for placebo subjects from the same cohort (n=9).

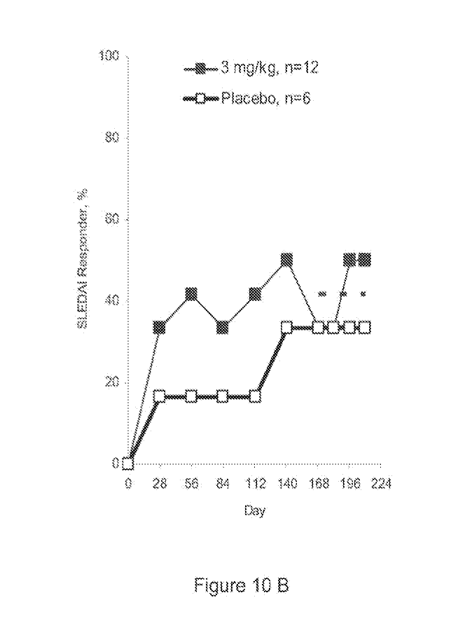

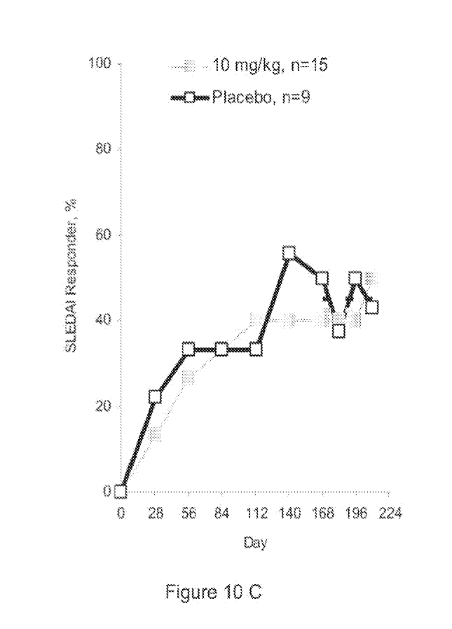

Data are from trial CP152. Subjects with moderately to severely active SLE were stratified by screening type I IFN signature and then exposed to placebo or sifalimumab at doses of 0.3, 1, 3, or 10 mg/kg IV every 2 weeks, from days 0-182. A SLEDAI response is shown, which is SLEDAI reduction (improvement in disease activity) >4 points, in subjects with baseline SLEDAI ≧6. Solid squares/line represent sifalimumab exposed subjects and open squares/dotted lines represent placebo patients. Short lines on days 168-210 only are average values for days 168-210, with no symbols, solid line representing average value for sifalimumab subjects and no symbols, dotted lines representing average values for placebo subjects. Subjects who received at least one dose of placebo or sifalimumab. Subjects who required rescue corticosteroids at levels greater than that allowed in the protocol for an increase in disease activity on or before day 196 are considered non-responders in this analysis. Subjects with a positive diagnostic test at baseline and with median reduction in type I IFN signature of <50% post dosing from days 28-210 are shown. FIG. 10 A includes subjects who received sifalimumab at 1 mg/kg IV with average SLEDAI response days 168-210 of 37% for sifalimumab subjects (n=9) and 21% for placebo subjects from the same cohort (n=7). FIG. 10 B includes subjects who received sifalimumab at 3 mg/kg IV with average SLEDAI response days 168-210 of 42% for sifalimumab subjects (n=12) and 33% for placebo subjects from the same cohort (n=6). FIG. 10 C includes subjects who received sifalimumab at 10 mg/kg IV with average SLEDAI response days 168-210 of 45% for sifalimumab subjects (n=15) and 45% for placebo subjects from the same cohort (n=9).

H. Delta Ct Relative to Fold-Change Relative to Healthy Donors

In the initial studies, we calculated fold changes using a pool of 24 normal healthy donors as a reference for each SLE patient. We have found, however, that this reference may have some confounding issues, specifically: 1) it can potentially introduce variance into the assay, and 2) manufacturing an artificial reference with the same dynamic range consistently is burdensome.

We conducted analyses to evaluate the need for this normal reference and found that when comparing delta Ct values to fold change values, there is greater than 99% correlation between the two (both log₂ transformed). Therefore, it is possible to use the delta Ct value with the house keeping genes from the SLE patient samples as the reference, and there is no need to measure or compare to healthy donors. This approach can be enhanced where the primary output of the diagnostic test is a qualitative result of positive or negative, as opposed to a quantitative score. Our results show that a cutoff of on the fold change scale translates to a cutoff of on the delta Ct scale.

### XI. Example 3

### Relationship Between Disease Activity and Type 1 Interferon- and Other Cytokine-Inducible Gene Expression in Blood in Dermatomyositis and Polymyositis

A. Background

Peripheral blood of 42 patients with DM or PM was subjected to gene expression profiling using Affymetrix human genome U133 plus 2.0 GeneChips® in an initial study to identify the prevalence of patients exhibiting periphery overexpression of type 1 IFN-inducible genes. To gain further scientific insight on type 1 IFN as a potential therapeutic target for DM and PM, 24 patients with DM or PM were then prospectively enrolled and followed for up to 6 years (mean of 1.9 years) while receiving standard clinical care.

B. Methodology

Clinical courses including MITAX (Myositis Intention to Treat Activity Index) scoring of disease activity were assessed across 150 patient visits. This index is based on 6 organs or systems that the physician uses, for each organ or system, which he/she would treat with large doses of steroids and/or immunosuppressive drugs. Peripheral blood samples collected at 80 patient visits were used for microarray analysis of cytokine-induced gene expression for type 1 IFN, TNF-α, IL-1β, GM-CSF, IL-10, and IL-13 signaling pathways.

C. Results

35 of 42 (87%) DM and PM patients had moderate/strong overexpression of type 1 IFN-inducible genes in the periphery blood. In the longitudinal study during the course of treatment, 21 of 24 patients showed overexpression of a type 1 IFN-inducible gene signature in peripheral blood. Specifically, overexpression of IFI27, IFI44, IFI44L, and RSAD2 and a type 1 IFN-inducible 13-gene composite signature (IFI27, RSAD2, IFI44L, ISG15, OAS3, HERC5, MX1, ESPTI1, IFIT3, IFI44, OAS1, IFIT1, and IFI6) correlated highly with disease activity during treatment. For 3 patients, type 1 IFN-inducible gene overexpression during treatment preceded disease relapse within approximately 1 month TNF-α, IL-1β, GM-CSF, IL-10, and IL-13 inducible gene signatures were also overexpressed in DM and PM patients but were not correlated with disease activity.

Targeting type 1 IFN may provide clinical benefit in DM and PM patient populations with overexpression of type 1 IFN-inducible genes in the periphery. Type 1 IFN-inducible gene overexpression in the periphery blood merits further study for use as a pharmacodynamic and predictive biomarker for developing anti-type 1 IFN therapy for these patients. Specifically, the results described above with SLE indicate that the four gene diagnostic will permit identification of DM and PM patients who will respond to anti-interferon alpha therapy.

### XII. Example 4

### Use of Four or Five Gene Signature to Evaluate the Presence and Magnitude of Overexpression of Type 1 IFN-Inducible Genes in Whole Blood from Patients Suffering from SLE, DM, PM, SSc, and RA

A. Background

We undertook studies to determine if there is a commonality of activation of the type I interferon (IFN) pathway in subjects with systemic lupus erythematosus (SLE), dermatomyositis (DM), polymyositis (PM), rheumatoid arthritis (RA), and systemic scleroderma (SSc).

We identified over-expressed transcripts in the whole blood (WB) of 262 SLE, 44 DM, 33 PM, 38 SSc and 89 RA subjects and compared expression to 24 healthy subjects using Affymetrix U133 Plus 2.0 Genechip®. Activation of the type I IFN pathway in WB was evaluated individually for each subject using the five gene type I IFN signature (IFI27, IFI44, IFI44L, IFI6, and RSAD2), as well as in lesional skin from 16 SLE, and 22 SSc subjects, muscle biopsies from 37 DM and 36 PM subjects, and synovium tissue from 20 RA subjects. Other cytokine gene signatures such as TNF-, IL1, IL-10, IL-13, IL-17, and GM-CSF were also assessed for pathway activation in the WB of these subjects. Additionally, a molecular classification of disease and healthy subjects was conducted with a clustering method using expression profiles of both type I IFN-inducible and non-type I IFN-inducible transcripts.

This 5 gene panel used to assess the activation of the type I IFN pathway in subjects was identified from the set of 21 type I IFN-inducible genes used to measure the pharmacodynamics of an anti-IFN-mAb in subjects with DM, PM, and SLE, as described in Yao et al., 2009. The 5 genes were found to be the most over-expressed among these 21 genes in subjects with SLE, DM, PM, SSc, RA, and Sjogren's disease compared to normal healthy donors (Table 3). The genes in Table 3 are sorted in descending for each block of 21 genes (within each individual disease), to illustrate the ranking of the 5 genes among the 21 genes.

TABLE 3
Gene expression in SLE, DM, PM, SSc, RA

| | | | | fold | | |
|---|---|---|---|---|---|---|
| Probe | Gene | Symbol | p-value | change | prevalence | disease |
| 202411_at | interferon, alpha-inducible protein 27 | IFI27 | 0 | 30.065 | 0.767 | SLE |
| 204415_at | interferon, alpha-inducible protein 6 | IFI6 | 0 | 16.465 | 0.882 | SLE |
| 214059_at | Interferon-induced protein 44 | IFI44 | 0 | 13.855 | 0.779 | SLE |
| 213797_at | radical S-adenosyl methionine domain containing 2 | RSAD2 | 0 | 13.232 | 0.763 | SLE |
| 204439_at | interferon-induced protein 44-like | IFI44L | 0 | 10.532 | 0.748 | SLE |
| 219211_at | ubiquitin specific peptidase 18 /// similar to ubiquitin | USP18 | 0 | 7.271 | 0.679 | SLE |
| | specific peptidase 18 /// similar to ubiquitin specific | | | | | |
| | peptidase 18 /// similar to ubiquitin specific peptidase 18 | | | | | |
| 44673_at | sialic acid binding Ig-like lectin 1, sialoadhesin | SIGLEC1 | 0 | 6.348 | 0.656 | SLE |
| 202145_at | lymphocyte antigen 6 complex, locus E | LY6E | 0 | 6.186 | 0.683 | SLE |
| 202869_at | 2′,5′-oligoadenylate synthetase 1, 40/46 kDa | OAS1 | 0 | 6.181 | 0.729 | SLE |
| 205569_at | lysosomal-associated membrane protein 3 | LAMP3 | 0 | 5.189 | 0.725 | SLE |
| 218400_at | 2′-5′-oligoadenylate synthetase 3, 100 kDa | OAS3 | 0 | 5.053 | 0.706 | SLE |
| 219863_at | hect domain and RLD 5 | HERC5 | 0 | 4.841 | 0.729 | SLE |
| 203153_at | interferon-induced protein with tetratricopeptide repeats 1 | IFIT1 | 0 | 4.840 | 0.729 | SLE |
| | /// interferon-induced protein with tetratricopeptide repeats | | | | | |
| | 1 | | | | | |
| 205483_s_at | ISG15 ubiquitin-like modifier | ISG15 | 0 | 4.633 | 0.702 | SLE |
| 227609_at | epithelial stromal interaction 1 (breast) | EPSTI1 | 0 | 4.519 | 0.706 | SLE |
| 202086_at | myxovirus (influenza virus) resistance 1, interferon- | MX1 | 0 | 4.142 | 0.725 | SLE |
| | inducible protein p78 (mouse) /// myxovirus (influenza | | | | | |
| | virus) resistance 1, interferon-inducible protein p78 | | | | | |
| | (mouse) | | | | | |
| 204972_at | 2′-5′-oligoadenylate synthetase 2, 69/71 kDa | OAS2 | 0 | 4.094 | 0.683 | SLE |
| 204747_at | interferon-induced protein with tetratricopeptide repeats 3 | IFIT3 | 0 | 3.992 | 0.702 | SLE |
| 219684_at | receptor (chemosensory) transporter protein 4 | RTP4 | 0 | 3.928 | 0.649 | SLE |
| 241916_at | Phospholipid scramblase 1 | PLSCR1 | 0 | 3.297 | 0.687 | SLE |
| 241812_at | DNA polymerase-transactivated protein 6 | DNAPTP6 | 0 | 3.217 | 0.561 | SLE |
| 204415_at1 | interferon, alpha-inducible protein 6 | IFI6 | 0 | 12.253 | 0.932 | DM |
| 214059_at1 | Interferon-induced protein 44 | IFI44 | 2.21E−11 | 9.736 | 0.750 | DM |
| 213797_at1 | radical S-adenosyl methionine domain containing 2 | RSAD2 | 5.63E−10 | 9.423 | 0.750 | DM |
| 202411_at1 | interferon, alpha-inducible protein 27 | IFI27 | 6.86E−06 | 8.796 | 0.682 | DM |
| 204439_at1 | interferon-induced protein 44-like | IFI44L | 3.13E−08 | 5.800 | 0.659 | DM |
| 202869_at1 | 2′,5′-oligoadenylate synthetase 1, 40/46 kDa | OAS1 | 2.91E−08 | 4.149 | 0.727 | DM |
| 203153_at1 | interferon-induced protein with tetratricopeptide repeats 1 | IFIT1 | 3.68E−06 | 3.883 | 0.682 | DM |
| | /// interferon-induced protein with tetratricopeptide repeats | | | | | |
| | 1 | | | | | |
| 219863_at1 | hect domain and RLD 5 | HERC5 | 3.03E−08 | 3.753 | 0.614 | DM |
| 205569_at1 | lysosomal-associated membrane protein 3 | LAMP3 | 2.11E−06 | 3.366 | 0.591 | DM |
| 204747_at1 | interferon-induced protein with tetratricopeptide repeats 3 | IFIT3 | 3.66E−09 | 3.263 | 0.682 | DM |
| 218400_at1 | 2′-5′-oligoadenylate synthetase 3, 100 kDa | OAS3 | 4.34E−06 | 3.191 | 0.591 | DM |
| 227609_at1 | epithelial stromal interaction 1 (breast) | EPSTI1 | 1.86E−06 | 3.096 | 0.568 | DM |
| 202086_at1 | myxovirus (influenza virus) resistance 1, interferon- | MX1 | 1.60E−06 | 3.058 | 0.636 | DM |
| | inducible protein p78 (mouse) /// myxovirus (influenza | | | | | |
| | virus) resistance 1, interferon-inducible protein p78 | | | | | |
| | (mouse) | | | | | |
| 219211_at1 | ubiquitin specific peptidase 18 /// similar to ubiquitin | USP18 | 5.25E−05 | 3.044 | 0.568 | DM |
| | specific peptidase 18 /// similar to ubiquitin specific | | | | | |
| | peptidase 18 /// similar to ubiquitin specific peptidase 18 | | | | | |
| 241916_at1 | Phospholipid scramblase 1 | PLSCR1 | 3.81E−08 | 2.939 | 0.705 | DM |
| 44673_at1 | sialic acid binding Ig-like lectin 1, sialoadhesin | SIGLEC1 | 2.87E−07 | 2.841 | 0.432 | DM |
| 205483_s_at1 | ISG15 ubiquitin-like modifier | ISG15 | 6.13E−06 | 2.722 | 0.568 | DM |
| 202145_at1 | lymphocyte antigen 6 complex, locus E | LY6E | 3.07E−05 | 2.685 | 0.500 | DM |
| 204972_at1 | 2′-5′-oligoadenylate synthetase 2, 69/71 kDa | OAS2 | 1.59E−06 | 2.545 | 0.500 | DM |
| 219684_at1 | receptor (chemosensory) transporter protein 4 | RTP4 | 3.84E−05 | 2.528 | 0.568 | DM |
| 241812_at1 | DNA polymerase-transactivated protein 6 | DNAPTP6 | 0.001397833 | 1.624 | 0.318 | DM |
| 204415_at2 | interferon, alpha-inducible protein 6 | IFI6 | 0 | 11.500 | 1.000 | PM |
| 213797_at2 | radical S-adenosyl methionine domain containing 2 | RSAD2 | 8.53E−08 | 6.775 | 0.727 | PM |
| 214059_at2 | Interferon-induced protein 44 | IFI44 | 4.66E−07 | 6.283 | 0.727 | PM |
| 202411_at2 | interferon, alpha-inducible protein 27 | IFI27 | 0.0003341 | 5.652 | 0.667 | PM |
| 204439_at2 | interferon-induced protein 44-like | IFI44L | 2.87E−06 | 4.470 | 0.667 | PM |
| 202869_at2 | 2′,5′-oligoadenylate synthetase 1, 40/46 kDa | OAS1 | 3.08E−08 | 4.431 | 0.697 | PM |
| 219863_at2 | hect domain and RLD 5 | HERC5 | 1.65E−06 | 3.102 | 0.576 | PM |
| 203153_at2 | interferon-induced protein with tetratricopeptide repeats 1 | IFIT1 | 0.000223959 | 2.922 | 0.576 | PM |
| | /// interferon-induced protein with tetratricopeptide repeats | | | | | |
| | 1 | | | | | |
| 227609_at2 | epithelial stromal interaction 1 (breast) | EPSTI1 | 2.60E−06 | 2.846 | 0.606 | PM |
| 205569_at2 | lysosomal-associated membrane protein 3 | LAMP3 | 6.22E−05 | 2.831 | 0.576 | PM |
| 241916_at2 | Phospholipid scramblase 1 | PLSCR1 | 3.24E−07 | 2.753 | 0.697 | PM |
| 204747_at2 | interferon-induced protein with tetratricopeptide repeats 3 | IFIT3 | 2.25E−06 | 2.684 | 0.606 | PM |
| 218400_at2 | 2′-5′-oligoadenylate synthetase 3, 100 kDa | OAS3 | 0.000152771 | 2.644 | 0.424 | PM |
| 44673_at2 | sialic acid binding Ig-like lectin 1, sialoadhesin | SIGLEC1 | 5.34E−05 | 2.620 | 0.424 | PM |
| 202145_at2 | lymphocyte antigen 6 complex, locus E | LY6E | 0.000183714 | 2.406 | 0.394 | PM |
| 205483_s_at2 | ISG15 ubiquitin-like modifier | ISG15 | 9.97E−05 | 2.369 | 0.485 | PM |
| 202086_at2 | myxovirus (influenza virus) resistance 1, interferon- | MX1 | 0.000540479 | 2.342 | 0.515 | PM |
| | inducible protein p78 (mouse) /// myxovirus (influenza | | | | | |
| | virus) resistance 1, interferon-inducible protein p78 | | | | | |
| | (mouse) | | | | | |
| 219684_at2 | receptor (chemosensory) transporter protein 4 | RTP4 | 8.87E−05 | 2.218 | 0.455 | PM |
| 219211_at2 | ubiquitin specific peptidase 18 /// similar to ubiquitin | USP18 | 0.003664023 | 2.195 | 0.364 | PM |
| | specific peptidase 18 /// similar to ubiquitin specific | | | | | |
| | peptidase 18 /// similar to ubiquitin specific peptidase 18 | | | | | |
| 204972_at2 | 2′-5′-oligoadenylate synthetase 2, 69/71 kDa | OAS2 | 0.000106864 | 2.082 | 0.364 | PM |
| 241812_at2 | DNA polymerase-transactivated protein 6 | DNAPTP6 | 0.0169138 | 1.533 | 0.242 | PM |
| 204415_at3 | interferon, alpha-inducible protein 6 | IFI6 | 0 | 6.235 | 0.865 | Rheumatoid Arthritis |
| 214059_at3 | Interferon-induced protein 44 | IFI44 | 1.60E−07 | 3.393 | 0.596 | Rheumatoid Arthritis |
| 202411_at3 | interferon, alpha-inducible protein 27 | IFI27 | 0.000442781 | 2.866 | 0.539 | Rheumatoid Arthritis |
| 213797_at3 | radical S-adenosyl methionine domain containing 2 | RSAD2 | 9.28E−06 | 2.783 | 0.494 | Rheumatoid Arthritis |
| 202869_at3 | 2′,5′-oligoadenylate synthetase 1, 40/46 kDa | OAS1 | 9.98E−08 | 2.391 | 0.528 | Rheumatoid Arthritis |
| 204439_at3 | interferon-induced protein 44-like | IFI44L | 5.15E−05 | 2.235 | 0.461 | Rheumatoid Arthritis |
| 202145_at3 | lymphocyte antigen 6 complex, locus E | LY6E | 5.88E−12 | 2.139 | 0.438 | Rheumatoid Arthritis |
| 241916_at3 | Phospholipid scramblase 1 | PLSCR1 | 1.66E−06 | 2.041 | 0.438 | Rheumatoid Arthritis |
| 44673_at3 | sialic acid binding Ig-like lectin 1, sialoadhesin | SIGLEC1 | 6.83E−08 | 1.908 | 0.326 | Rheumatoid Arthritis |
| 227609_at3 | epithelial stromal interaction 1 (breast) | EPSTI1 | 6.66E−06 | 1.825 | 0.360 | Rheumatoid Arthritis |
| 204972_at3 | 2′-5′-oligoadenylate synthetase 2, 69/71 kDa | OAS2 | 1.23E−09 | 1.761 | 0.315 | Rheumatoid Arthritis |
| 202086_at3 | myxovirus (influenza virus) resistance 1, interferon- | MX1 | 0.000342238 | 1.739 | 0.371 | Rheumatoid Arthritis |
| | inducible protein p78 (mouse) /// myxovirus (influenza | | | | | |
| | virus) resistance 1, interferon-inducible protein p78 | | | | | |
| | (mouse) | | | | | |
| 218400_at3 | 2′-5′-oligoadenylate synthetase 3, 100 kDa | OAS3 | 0.000277383 | 1.739 | 0.371 | Rheumatoid Arthritis |
| 219863_at3 | hect domain and RLD 5 | HERC5 | 0.000175256 | 1.692 | 0.303 | Rheumatoid Arthritis |
| 205569_at3 | lysosomal-associated membrane protein 3 | LAMP3 | 0.0029278 | 1.630 | 0.303 | Rheumatoid Arthritis |
| 219684_at3 | receptor (chemosensory) transporter protein 4 | RTP4 | 0.00252807 | 1.544 | 0.281 | Rheumatoid Arthritis |
| 204747_at3 | interferon-induced protein with tetratricopeptide repeats 3 | IFIT3 | 0.005459384 | 1.445 | 0.348 | Rheumatoid Arthritis |
| 205483_s_at3 | ISG15 ubiquitin-like modifier | ISG15 | 0.009827494 | 1.393 | 0.292 | Rheumatoid Arthritis |
| 219211_at3 | ubiquitin specific peptidase 18 /// similar to ubiquitin | USP18 | 0.079726231 | 1.274 | 0.236 | Rheumatoid Arthritis |
| | specific peptidase 18 /// similar to ubiquitin specific | | | | | |
| | peptidase 18 /// similar to ubiquitin specific peptidase 18 | | | | | |
| 241812_at3 | DNA polymerase-transactivated protein 6 | DNAPTP6 | 0.121840941 | 1.137 | 0.124 | Rheumatoid Arthritis |
| 203153_at3 | interferon-induced protein with tetratricopeptide repeats 1 | IFIT1 | 0.654268059 | 1.093 | 0.270 | Rheumatoid Arthritis |
| | /// interferon-induced protein with tetratricopeptide repeats | | | | | |
| | 1 | | | | | |
| 204415_at4 | interferon, alpha-inducible protein 6 | IFI6 | 0 | 15.488 | 1.000 | Scleroderma |
| 213797_at4 | radical S-adenosyl methionine domain containing 2 | RSAD2 | 1.92E−08 | 5.758 | 0.711 | Scleroderma |
| 214059_at4 | Interferon-induced protein 44 | IFI44 | 2.52E−07 | 4.494 | 0.658 | Scleroderma |
| 202411_at4 | interferon, alpha-inducible protein 27 | IFI27 | 0.001868066 | 4.096 | 0.579 | Scleroderma |
| 202869_at4 | 2′,5′-oligoadenylate synthetase 1, 40/46 kDa | OAS1 | 1.43E−08 | 3.581 | 0.711 | Scleroderma |
| 204439_at4 | interferon-induced protein 44-like | IFI44L | 1.98E−05 | 3.105 | 0.553 | Scleroderma |
| 203153_at4 | interferon-induced protein with tetratricopeptide repeats 1 | IFIT1 | 2.36E−05 | 2.919 | 0.632 | Scleroderma |
| | /// interferon-induced protein with tetratricopeptide repeats | | | | | |
| | 1 | | | | | |
| 219863_at4 | hect domain and RLD 5 | HERC5 | 2.37E−08 | 2.860 | 0.632 | Scleroderma |
| 202086_at4 | myxovirus (influenza virus) resistance 1, interferon- | MX1 | 9.86E−06 | 2.308 | 0.474 | Scleroderma |
| | inducible protein p78 (mouse) /// myxovirus (influenza | | | | | |
| | virus) resistance 1, interferon-inducible protein p78 | | | | | |
| | (mouse) | | | | | |
| 218400_at4 | 2′-5′-oligoadenylate synthetase 3, 100 kDa | OAS3 | 3.26E−05 | 2.300 | 0.474 | Scleroderma |
| 204747_at4 | interferon-induced protein with tetratricopeptide repeats 3 | IFIT3 | 8.63E−07 | 2.280 | 0.526 | Scleroderma |
| 202145_at4 | lymphocyte antigen 6 complex, locus E | LY6E | 2.38E−05 | 2.229 | 0.421 | Scleroderma |
| 205569_at4 | lysosomal-associated membrane protein 3 | LAMP3 | 0.0001192 | 2.206 | 0.500 | Scleroderma |
| 205483_s_at4 | ISG15 ubiquitin-like modifier | ISG15 | 1.45E−05 | 2.177 | 0.447 | Scleroderma |
| 227609_at4 | epithelial stromal interaction 1 (breast) | EPSTI1 | 2.54E−05 | 2.177 | 0.553 | Scleroderma |
| 219684_at4 | receptor (chemosensory) transporter protein 4 | RTP4 | 0.000226923 | 1.943 | 0.395 | Scleroderma |
| 204972_at4 | 2′-5′-oligoadenylate synthetase 2, 69/71 kDa | OAS2 | 3.34E−06 | 1.909 | 0.368 | Scleroderma |
| 44673_at4 | sialic acid binding Ig-like lectin 1, sialoadhesin | SIGLEC1 | 0.000242727 | 1.831 | 0.342 | Scleroderma |
| 219211_at4 | ubiquitin specific peptidase 18 /// similar to ubiquitin | USP18 | 0.002152959 | 1.815 | 0.316 | Scleroderma |
| | specific peptidase 18 /// similar to ubiquitin specific | | | | | |
| | peptidase 18 /// similar to ubiquitin specific peptidase 18 | | | | | |
| 241916_at4 | Phospholipid scramblase 1 | PLSCR1 | 0.000483597 | 1.793 | 0.395 | Scleroderma |
| 241812_at4 | DNA polymerase-transactivated protein 6 | DNAPTP6 | 0.303084481 | 0.913 | 0.079 | Scleroderma |
| 202411_at | interferon, alpha-inducible protein 27 | IFI27 | 0.00094654 | 25.635 | 1.000 | Sjogren Syndrome |
| 204439_at | interferon-induced protein 44-like | IFI44L | 0.008982765 | 5.742 | 1.000 | Sjogren Syndrome |
| 219211_at | ubiquitin specific peptidase 18 /// similar to ubiquitin | USP18 | 0.002566614 | 5.079 | 1.000 | Sjogren Syndrome |
| | specific peptidase 18 /// similar to ubiquitin specific | | | | | |
| | peptidase 18 /// similar to ubiquitin specific peptidase 18 | | | | | |
| 213797_at | radical S-adenosyl methionine domain containing 2 | RSAD2 | 0.003104168 | 4.705 | 1.000 | Sjogren Syndrome |
| 203153_at | interferon-induced protein with tetratricopeptide repeats 1 | IFIT1 | 0.0104006 | 4.142 | 1.000 | Sjogren Syndrome |
| | /// interferon-induced protein with tetratricopeptide repeats | | | | | |
| | 1 | | | | | |
| 204747_at | interferon-induced protein with tetratricopeptide repeats 3 | IFIT3 | 0.003638823 | 3.857 | 1.000 | Sjogren Syndrome |
| 44673_at | sialic acid binding Ig-like lectin 1, sialoadhesin | SIGLEC1 | 0.007125541 | 3.755 | 1.000 | Sjogren Syndrome |
| 205483_s_at | ISG15 ubiquitin-like modifier | ISG15 | 0.000690576 | 3.557 | 1.000 | Sjogren Syndrome |
| 219863_at | hect domain and RLD 5 | HERC5 | 0.007639108 | 3.499 | 1.000 | Sjogren Syndrome |
| 227609_at | epithelial stromal interaction 1 (breast) | EPSTI1 | 0.001244694 | 3.060 | 1.000 | Sjogren Syndrome |
| 204415_at | interferon, alpha-inducible protein 6 | IFI6 | 0.000905658 | 2.912 | 1.000 | Sjogren Syndrome |
| 218400_at | 2′-5′-oligoadenylate synthetase 3, 100 kDa | OAS3 | 0.009517756 | 2.764 | 1.000 | Sjogren Syndrome |
| 202086_at | myxovirus (influenza virus) resistance 1, interferon- | MX1 | 0.003908144 | 2.722 | 1.000 | Sjogren Syndrome |
| | inducible protein p78 (mouse) /// myxovirus (influenza | | | | | |
| | virus) resistance 1, interferon-inducible protein p78 | | | | | |
| | (mouse) | | | | | |
| 204972_at | 2′-5′-oligoadenylate synthetase 2, 69/71 kDa | OAS2 | 0.00753208 | 2.590 | 1.000 | Sjogren Syndrome |
| 214059_at | Interferon-induced protein 44 | IFI44 | 0.039282229 | 2.477 | 0.750 | Sjogren Syndrome |
| 202145_at | lymphocyte antigen 6 complex, locus E | LY6E | 0.01319652 | 2.418 | 0.750 | Sjogren Syndrome |
| 202869_at | 2′,5′-oligoadenylate synthetase 1, 40/46 kDa | OAS1 | 0.022562072 | 1.796 | 0.250 | Sjogren Syndrome |
| 219684_at | receptor (chemosensory) transporter protein 4 | RTP4 | 0.231709806 | 1.749 | 0.250 | Sjogren Syndrome |
| 241916_at | Phospholipid scramblase 1 | PLSCR1 | 0.472321498 | 1.284 | 0.000 | Sjogren Syndrome |
| 241812_at | DNA polymerase-transactivated protein 6 | DNAPTP6 | 0.529363363 | 0.917 | 0.000 | Sjogren Syndrome |
| 205569_at | lysosomal-associated membrane protein 3 | LAMP3 | 0.318182147 | 0.905 | 0.000 | Sjogren Syndrome |

We found that the proportion of SLE, DM, PM, SSc, and RA subjects that were positive for activation of the type I IFN pathway in WB was 72% for SLE, 66% for DM, 61% for PM, 50% for SSc, and 33% for RA. The concordant overexpression of type I IFN-inducible transcripts was observed in both involved tissue and WB.

From these studies, consistently observed activation of the type I IFN pathway in a population of SLE, DM, PM, SSc, and RA subjects. Subgroups of PM and RA subjects also showed activation of the TNF-pathway and a subgroup of SSc subjects showed strong activation of the IL-17 pathway. A common set of 36 type I IFN-inducible transcripts were among the most over-expressed transcripts in the WB of subjects. Thus, these studies show that it is possible to classify all subjects with different disease manifestations by molecular features, rather than classic SLE, DM, PM, SSc, and RA nomenclature is presented.

B. Results

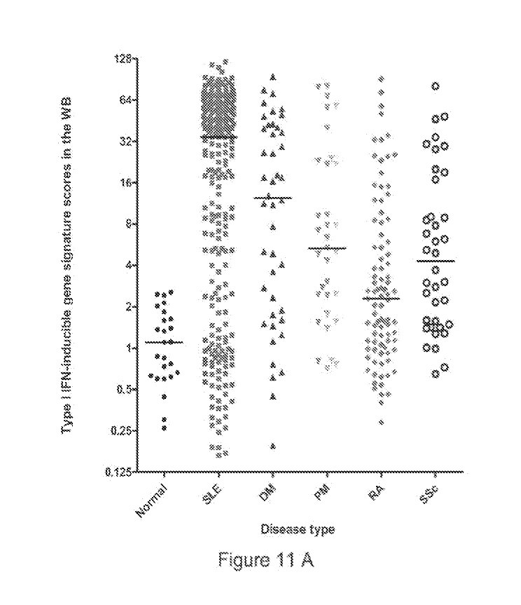

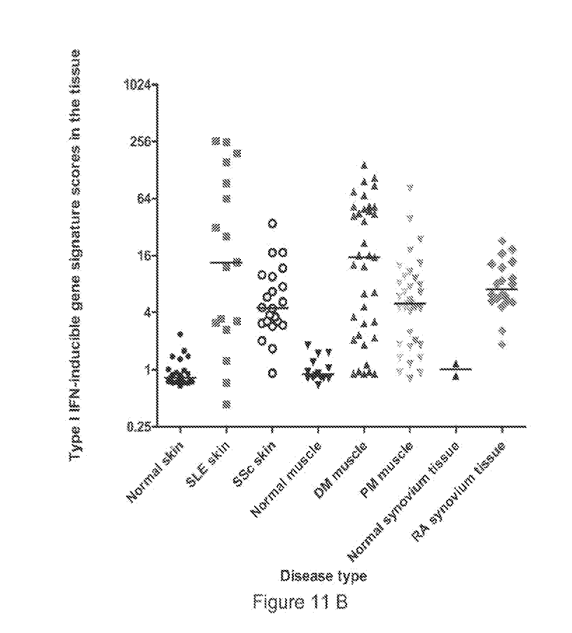

The scores for the type I IFN-inducible gene signatures as calculated using the five type I IFN-inducible genes in the WB of individual SLE, DM, PM, SSc, and RA subjects and healthy subjects are shown in FIG. 11 A. Composite scores of the relative expression of multiple genes can provide a robust measurement of pathway activity. Patients with the composite scores of more than 4 were considered type I IFN-inducible gene signature positive. The threshold for type I IFN gene signature positive/negative status using the 5 gene panel was determined using the distribution of signature values from the healthy normal donors. Since the sample size for these 24 normal healthy donors is modest, we built in a conservative estimate for a signature status threshold. The maximum of the type I IFN gene signature values in the normal healthy donors is 3. A cut off of 4 allows for additional variance that is likely more aligned with the general population. The scores for the overexpression of type I IFN-inducible gene signatures in the WB of these subjects were significant when compared to that of healthy subjects (median scores for healthy normal, SLE, DM, PM, RA and SSc subjects are 1.1, 34.5, 12.4, 5.3, 2.3, and 4.3, respectively; p-values are 7.8×10⁻⁴⁷, 7.5×10⁻⁷, 6.6×10⁻⁴, 4.7×10⁻⁵, and 5.2×10⁻⁴, for SLE, DM, PM, RA, and SSc subjects, respectively).

For each autoimmune disease, the percentage of subjects that were scored as “signature positive” for activation of type I IFN pathways was using the 5 gene type I IFN signature score (Table 4). Patients that are signature positive for activation of the type 1 IFN pathway within each autoimmune disease are: 73% SLE, 66% DM, 61% PM, and 50% SSc, 33% RA. This method for identifying similar subjects with a positive signature (or negative signature) suggests a robust presence of type I IFN gene overexpression in a specific subgroup of these autoimmune diseases.

TABLE 4
Percentages of subjects with a positive type I IFN-
inducible gene signature in normal healthy controls
and SLE, DM, PM, RA, and SSc diseases in the WB

| | Normal | | | | | |
|---|---|---|---|---|---|---|
| Signature | donors | SLE | DM | PM | RA | SSc |
| Type I IFN | 0.0% | 72.5% | 65.9% | 60.6% | 32.6% | 50.0% |
| signature | | | | | | |
| Sample size | 24 | 262 | 44 | 33 | 89 | 38 |

We also evaluated the overexpression of type I IFN-inducible genes in the disease tissues (lesional skin of SLE and SSc subjects, muscle specimen of DM and PM subjects, and synovium tissue of RA subjects). We used in vitro stimulation of a primary human keratinocyte/fibroblast model and myoblast muscle cell line (SkMC) with IFN- or IFN- to identify potential type I IFN-inducible transcripts in cells characteristic of the skin and muscle. Most of the transcripts induced by type I IFN in the resident skin cells or muscle cell lines were also inducible in the WB (skin data was described in Yao Y, Jallal J, et al. Type I IFN as a potential therapeutic target for psoriasis. PLoS ONE 2008; 3(7): e2737. doi:10.1371/journal.pone.0002737.) In the following sample sizes, 16 SLE, 37 DM, 36 PM, 22 SSc, and 20 RA subjects, a high overexpression of type I IFN-inducible genes was observed at the disease sites in a large subset of SLE, SSc, DM, PM, and RA subjects that were evaluated (p-values <0.01 for SLE, SSc, DM, and PM subjects, respectively; median 5 gene type I IFN signature scores are 0.8, 13.6, 4.5 in skin of normal healthy donors, SLE and SSc subjects; 0.9, 15.3, and 5 in muscle specimen of normal health donors, DM and PM subjects; 1.0 and 7.1 in synovial tissues and normal healthy donor and RA subjects (the RA comparison had too small of a normal control sample size to evaluate); FIG. 11 B).

In an analysis between those patients that have positive type I IFN-inducible gene signatures (using the 5 gene signature) in matched WB and disease tissue specimens, 13 of 16 SLE subjects showed comparable type I IFN-inducible gene signature scores in WB and lesional skin (P<0.05 using Fisher's exact test). For the 10 DM and 9 PM subjects with paired WB and muscle specimens, 2 DM muscle specimens, 1 PM muscle and 1 PM WB specimen (from the same patient) were negative for a type I IFN-inducible gene signature. For the 10 SSc subjects with paired WB and skin specimens, 7 subjects demonstrated comparable type I IFN-inducible gene signature scores. These observations show a strong trend of concordant expression of type I IFN-inducible genes in the WB and disease tissues of SLE, DM, PM, and SSc subjects.

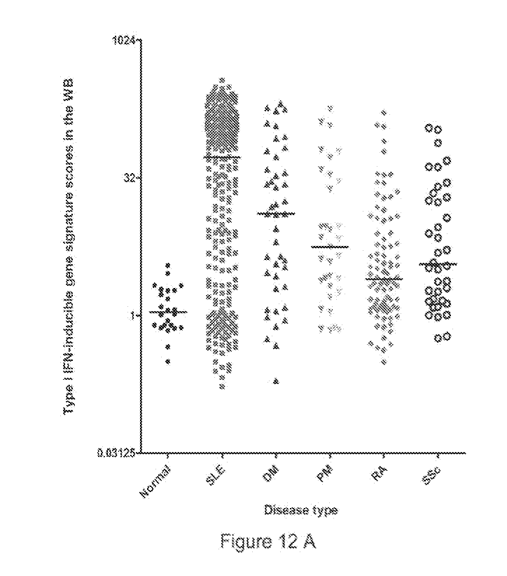

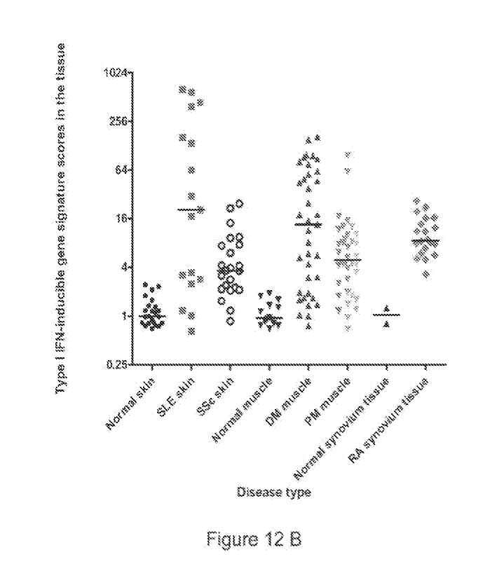

In a further analysis, we studied the four gene signature (IFI27, IFI44, IFI44L, and RSAD2) and determined it also shows a strong trend of concordant expression of the four gene type I IFN-inducible genes in the WB and disease tissues of SLE, DM, PM, and SSc subjects. See FIGS. 12 A and B.

1-33. (canceled)

34. A method for treating lupus, comprising:
administering an antibody, or antigen binding portion thereof, to a subject identified as having an increase in mRNA of genes consisting of
interferon-induced protein 44 (IFI44),
radical S-adenosyl methionine domain containing 2 (RSAD2),
interferon-induced protein 44-like (IFI44L) and
interferon alpha-inducible protein 27 (IFI27)
in a biological sample, thereby treating the lupus,
wherein:
the antibody, or antigen binding portion thereof, comprises:
(a) a heavy chain variable region CDR1 comprising SEQ ID NO: 36,
(b) a heavy chain variable region CDR2 comprising SEQ ID NO: 37,
(c) a heavy chain variable region CDR3 comprising SEQ ID NO: 38,
(d) a light chain variable region CDR1 comprising SEQ ID NO: 39,
(e) a light chain variable region CDR2 comprising SEQ ID NO: 40, and
(f) a light chain variable region CDR3 comprising SEQ ID NO: 41; and
the increase in mRNA of the genes is at least about 4-fold relative to pooled samples from healthy subjects.

35. The method of claim 34, wherein the antibody, or antigen binding portion thereof, comprises a heavy chain variable region comprising SEQ ID NO: 42 and a light chain variable region comprising SEQ ID NO: 43.

36. The method of claim 34, wherein an antibody is administered to the subject and the antibody comprises:
(a) a heavy chain variable region CDR1 comprising SEQ ID NO: 36;
(b) a heavy chain variable region CDR2 comprising SEQ ID NO: 37;
(c) a heavy chain variable region CDR3 comprising SEQ ID NO: 38;
(d) a light chain variable region CDR1 comprising SEQ ID NO: 39;
(e) a light chain variable region CDR2 comprising SEQ ID NO: 40; and
(f) a light chain variable region CDR3 comprising SEQ ID NO: 41.

37. The method of claim 34, wherein an antibody is administered to the subject and the antibody comprises a heavy chain variable region comprising SEQ ID NO: 42 and a light chain variable region comprising SEQ ID NO: 43.

38. The method of claim 34, wherein the increase in the mRNA of the genes is an average increase in the mRNA of the genes.

39. The method of claim 38, wherein the average increase is a mean increase or median increase.

40. The method of claim 34, wherein the increase is determined relative to one or more control genes present in the sample, and the threshold is equivalent to the threshold utilized when the increase is determined relative to pooled samples from healthy patients.

41. The method of claim 40, wherein the one or more control genes are chosen from ACTB, GAPDH, and 18S rRNA.

42. The method of claim 34, wherein the sample is whole blood or serum.

43. The method of claim 36, wherein the sample is whole blood or serum.

44. The method of claim 37, wherein the sample is whole blood or serum.

45. The method of claim 34, wherein the subject is in need of treatment of systemic lupus erythematosus.

46. The method of claim 36, wherein the subject is in need of treatment of systemic lupus erythematosus.

47. The method of claim 37, wherein the subject is in need of treatment of systemic lupus erythematosus.

48. The method of claim 34, which comprises detecting the mRNA levels of the genes in the sample from the subject.

49. The method of claim 48, comprising:
(i) isolating RNA from the sample;
(ii) synthesizing cDNA from the RNA;
(iii) annealing the cDNA with oligonucleotides that hybridize to polynucleotides chosen from SEQ ID NOs: 25-28 and 32, and
(iv) amplifying the cDNA and detecting the amplified products.

50. The method of claim 49, wherein the oligonucleotides are chosen from oligonucleotides having the sequences of SEQ ID NOs: 13-24.

51. The method of claim 37, which comprises detecting the mRNA levels of the genes in the sample from the subject.

52. The method of claim 51, comprising:
(i) isolating RNA from the sample;
(ii) synthesizing cDNA from the RNA;
(iii) annealing the cDNA with oligonucleotides that hybridize to polynucleotides chosen from SEQ ID NOs: 25-28 and 32, and
(iv) amplifying the cDNA and detecting the amplified products.

53. The method of claim 52, wherein the oligonucleotides are chosen from oligonucleotides having the sequences of SEQ ID NOs: 13-24.
